SEQUENCE LISTING (104 sequences)

SEQ ID NO: 1 - protein, 5 aa, synthetic construct
  REGION 1..5: note = synthetic
SYAIS

SEQ ID NO: 2 - protein, 5 aa, synthetic construct
  REGION 1..5: note = synthetic
SYGIH

SEQ ID NO: 3 - protein, 6 aa, synthetic construct
  REGION 1..6: note = synthetic
SSNWWS

SEQ ID NO: 4 - protein, 5 aa, synthetic construct
  REGION 1..5: note = synthetic
SYGIS

SEQ ID NO: 5 - protein, 5 aa, synthetic construct
  REGION 1..5: note = synthetic
SYGMH

SEQ ID NO: 6 - protein, 5 aa, synthetic construct
  REGION 1..5: note = synthetic
DYGIS

SEQ ID NO: 7 - protein, 5 aa, synthetic construct
  REGION 1..5: note = synthetic
TYVTS

SEQ ID NO: 8 - protein, 17 aa, synthetic construct
  REGION 1..17: note = synthetic
GIIPIFGTANYAQKFQG

SEQ ID NO: 9 - protein, 17 aa, synthetic construct
  REGION 1..17: note = synthetic
IVSYDGRHKYYADSVKG

SEQ ID NO: 10 - protein, 16 aa, synthetic construct
  REGION 1..16: note = synthetic
EVYHSGSTNYNPSLKS

SEQ ID NO: 11 - protein, 17 aa, synthetic construct
  REGION 1..17: note = synthetic
WINTYNGNTNYAQKLQG

SEQ ID NO: 12 - protein, 17 aa, synthetic construct
  REGION 1..17: note = synthetic
WISAYNGNTNYAQKLQG

SEQ ID NO: 13 - protein, 17 aa, synthetic construct
  REGION 1..17: note = synthetic
VISYDGSNKYYADSVKG

SEQ ID NO: 14 - protein, 17 aa, synthetic construct
  REGION 1..17: note = synthetic
WISGDNVKTTYAKKFQG

SEQ ID NO: 15 - protein, 17 aa, synthetic construct
  REGION 1..17: note = synthetic
WISAYNGNTYYAQKLQG

SEQ ID NO: 16 - protein, 17 aa, synthetic construct
  REGION 1..17: note = synthetic
GIIPMFDTTEYAQKLEG

SEQ ID NO: 17 - protein, 15 aa, synthetic construct
  REGION 1..15: note = synthetic
DYYDSSGYYFDAFDI

SEQ ID NO: 18 - protein, 13 aa, synthetic construct
  REGION 1..13: note = synthetic
DSGNSGLEGIHDY

SEQ ID NO: 19 - protein, 19 aa, synthetic construct
  REGION 1..19: note = synthetic
NIPLSSSWPNYYYYYGMDV

SEQ ID NO: 20 - protein, 19 aa, synthetic construct
  REGION 1..19: note = synthetic
DIGYSGSYYSPYYYYGMDV

SEQ ID NO: 21 - protein, 21 aa, synthetic construct
  REGION 1..21: note = synthetic
VPSEYYDFWSGYYTEENAFDI

SEQ ID NO: 22 - protein, 15 aa, synthetic construct
  REGION 1..15: note = synthetic
DNGDYGYYYYYYMDV

SEQ ID NO: 23 - protein, 7 aa, synthetic construct
  REGION 1..7: note = synthetic
GGIVFDY

SEQ ID NO: 24 - protein, 10 aa, synthetic construct
  REGION 1..10: note = synthetic
LSYYNYAMDV

SEQ ID NO: 25 - protein, 9 aa, synthetic construct
  REGION 1..9: note = synthetic
AGGLSHFDY

SEQ ID NO: 26 - protein, 11 aa, synthetic construct
  REGION 1..11: note = synthetic
VKWELGWAFDI

SEQ ID NO: 27 - protein, 13 aa, synthetic construct
  REGION 1..13: note = synthetic
SGSSSNIGTNTVS

SEQ ID NO: 28 - protein, 11 aa, synthetic construct
  REGION 1..11: note = synthetic
RASQGISSWLA

SEQ ID NO: 29 - protein, 11 aa, synthetic construct
  REGION 1..11: note = synthetic
QGDSLRNYYAS

SEQ ID NO: 30 - protein, 11 aa, synthetic construct
  REGION 1..11: note = synthetic
QGDGLRNYFAS

SEQ ID NO: 31 - protein, 11 aa, synthetic construct
  REGION 1..11: note = synthetic
RASQSISSYLN

SEQ ID NO: 32 - protein, 11 aa, synthetic construct
  REGION 1..11: note = synthetic
RASQSISRWLA

SEQ ID NO: 33 - protein, 12 aa, synthetic construct
  REGION 1..12: note = synthetic
RASQSVSGNYLA

SEQ ID NO: 34 - protein, 14 aa, synthetic construct
  REGION 1..14: note = synthetic
TGTSSDVGGYNHVS

SEQ ID NO: 35 - protein, 16 aa, synthetic construct
  REGION 1..16: note = synthetic
RSSQSLLHSNGNNYLN

SEQ ID NO: 36 - protein, 14 aa, synthetic construct
  REGION 1..14: note = synthetic
TGSSSNIGAGYDVH

SEQ ID NO: 37 - protein, 7 aa, synthetic construct
  REGION 1..7: note = synthetic
NNNERPS

SEQ ID NO: 38 - protein, 7 aa, synthetic construct
  REGION 1..7: note = synthetic
AASSLQS

SEQ ID NO: 39 - protein, 7 aa, synthetic construct
  REGION 1..7: note = synthetic
GKNNRPS

SEQ ID NO: 40 - protein, 7 aa, synthetic construct
  REGION 1..7: note = synthetic
DASSLES

SEQ ID NO: 41 - protein, 7 aa, synthetic construct
  REGION 1..7: note = synthetic
GASSRAT

SEQ ID NO: 42 - protein, 7 aa, synthetic construct
  REGION 1..7: note = synthetic
EVSNRPS

SEQ ID NO: 43 - protein, 7 aa, synthetic construct
  REGION 1..7: note = synthetic
LSSRRAS

SEQ ID NO: 44 - protein, 7 aa, synthetic construct
  REGION 1..7: note = synthetic
GNSNRPS

SEQ ID NO: 45 - protein, 11 aa, synthetic construct
  REGION 1..11: note = synthetic
AAWDDNLTGLV

SEQ ID NO: 46 - protein, 9 aa, synthetic construct
  REGION 1..9: note = synthetic
QQANSFPLT

SEQ ID NO: 47 - protein, 11 aa, synthetic construct
  REGION 1..11: note = synthetic
NSRDSSAKHWV

SEQ ID NO: 48 - protein, 11 aa, synthetic construct
  REGION 1..11: note = synthetic
NSRDGSGKHLV

SEQ ID NO: 49 - protein, 8 aa, synthetic construct
  REGION 1..8: note = synthetic
QQSYSTWT

SEQ ID NO: 50 - protein, 9 aa, synthetic construct
  REGION 1..9: note = synthetic
QQSYSTPLT

SEQ ID NO: 51 - protein, 9 aa, synthetic construct
  REGION 1..9: note = synthetic
QQYGSSPGT

SEQ ID NO: 52 - protein, 10 aa, synthetic construct
  REGION 1..10: note = synthetic
SSYTSSNTWV

SEQ ID NO: 53 - protein, 9 aa, synthetic construct
  REGION 1..9: note = synthetic
MQGLQTPWT

SEQ ID NO: 54 - protein, 10 aa, synthetic construct
  REGION 1..10: note = synthetic
QSYDSSLSTV

SEQ ID NO: 55 - protein, 124 aa, synthetic construct
  REGION 1..124: note = synthetic
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDYYDSSGYYFDAFDIWGQGTMVTVSS

SEQ ID NO: 56 - protein, 122 aa, synthetic construct
  REGION 1..122: note = synthetic
QVQLVQSGGGVVQPGRSLRLSCAASGFTFSSYGIHWVRQAPGKGLEWVAIVSYDGRHKYYADSVKGRFTISRDDSKNTIYLQMDSLRAEDTAVYYCAKDSGNSGLEGIHDYWGQGTLVTVSS

SEQ ID NO: 57 - protein, 128 aa, synthetic construct
  REGION 1..128: note = synthetic
QVQLQESGPGLVKPSGTLSLTCAVSGGSISSSNWWSWVRQPPGKGLEWIGEVYHSGSTNYNPSLKSRVTISVDKSKNQFSLKLSSVTAADTAVYYCARNIPLSSSWPNYYYYYGMDVWGQGTTVTVSS

SEQ ID NO: 58 - protein, 128 aa, synthetic construct
  REGION 1..128: note = synthetic
QVQLVQSGAEVKKPGASVKVSCKASGYTFTSYGISWVRQAPGQGLEWMGWINTYNGNTNYAQKLQGRVTMTTDTSTTTAYMELRSLRSDDTAVYYCARDIGYSGSYYSPYYYYGMDVWGQGTTVTVSS

SEQ ID NO: 59 - protein, 130 aa, synthetic construct
  REGION 1..130: note = synthetic
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVPSEYYDFWSGYYTEENAFDIWGQGTMVTVSS

SEQ ID NO: 60 - protein, 124 aa, synthetic construct
  REGION 1..124: note = synthetic
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDNGDYGYYYYYYMDVWGKGTTVTVSS

SEQ ID NO: 61 - protein, 116 aa, synthetic construct
  REGION 1..116: note = synthetic
QVQLVQSGAEVKKPGASVKVSCKASGYTFSDYGISWVRQAPGQGLEWMGWISGDNVKTTYAKKFQGRVTLTTDTSTSTAYMELRSLTSDDTAAYYCARGGIVFDYWGQGTLVTVSS

SEQ ID NO: 62 - protein, 116 aa, synthetic construct
  REGION 1..116: note = synthetic
QVQLVQSGAEVKKPGASVKVSCKASGYTFTSYGISWVRQATGQGLEWMGWISAYNGNTNYAQKLQGRVTMTTDTSTSTAYMELRSLRSDDTAVYYCARGGIVFDYWGQGTLVTVSS

SEQ ID NO: 63 - protein, 119 aa, synthetic construct
  REGION 1..119: note = synthetic
QMQLVQSGAEVKMPGASVKVSCKASGYTFTSYGISWVRQAPGQGLEWMGWISAYNGNTYYAQKLQGRVTMTADTSTSTAYMDLRSLRSDDTAVYYCARLSYYNYAMDVWGQGTTVTVSS

SEQ ID NO: 64 - protein, 118 aa, synthetic construct
  REGION 1..118: note = synthetic
QVQLQQSGAEVKKPGSSVKVSCKASGGIFSTYVTSWVRQAPGQGLEWMGGIIPMFDTTEYAQKLEGRVTITVDESTNTAYMELSSLRFEDTAVYYCARAGGLSHFDYWGQGTLVTVSS

SEQ ID NO: 65 - protein, 120 aa, synthetic construct
  REGION 1..120: note = synthetic
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVKWELGWAFDIWGQGTMVTVSS

SEQ ID NO: 66 - protein, 110 aa, synthetic construct
  REGION 1..110: note = synthetic
QAGLTQPPSASGTPGQRVIISCSGSSSNIGTNTVSWYQHLPGTAPKLLIYNNNERPSGVPRRFSGSRSGASASLAISGLQSDDEAHYYCAAWDDNLTGLVFGGGTKLTVL

SEQ ID NO: 67 - protein, 107 aa, synthetic construct
  REGION 1..107: note = synthetic
DVVMTQSPSSVSASVGDRVTITCRASQGISSWLAWYQQKPGKAPKLLIYAASSLQSGVPSRFSGSGSGTDFTLTISSLQPEDFGTYYCQQANSFPLTFGGGTKVEIK

SEQ ID NO: 68 - protein, 107 aa, synthetic construct
  REGION 1..107: note = synthetic
SSELTQDPAVSVALGQTVRITCQGDSLRNYYASWYQQKPGQAPVLVIYGKNNRPSGIPDRFSGSSSGNTASLTINGAQAEDEADYYCNSRDSSAKHWVFGGGTKLTV

SEQ ID NO: 69 - protein, 107 aa, synthetic construct
  REGION 1..107: note = synthetic
SSELTQDPAVSVALGQTVRITCQGDGLRNYFASWYQQKPGQAPVLVIYGKNNRPSGIPDRFSGSSSGNTASLRIAGAQAEDEADYYCNSRDGSGKHLVFGGGTKLTV

SEQ ID NO: 70 - protein, 106 aa, synthetic construct
  REGION 1..106: note = synthetic
DIVMTQSPSSLSASVGDRVTITCRASQSISSYLNWYQQKPGKAPKLLIYAASSLQSGVPSRFSGSGSGTDFTLTISSLQPEDFATYYCQQSYSTWTFGQGTKVEIK

SEQ ID NO: 71 - protein, 107 aa, synthetic construct
  REGION 1..107: note = synthetic
DIQMTQSPSTLSASVGDRVTITCRASQSISRWLAWYQQKPGKAPKLLIYDASSLESGVPSRFSGSGSGTEFTLTISSLQPDDFATYYCQQSYSTPLTFGPGTKVDIK

SEQ ID NO: 72 - protein, 108 aa, synthetic construct
  REGION 1..108: note = synthetic
ETTLTQSPGTLSLSPGERATLSCRASQSVSGNYLAWYQQKPGQPPRLLIFGASSRATGIPDRFSGSGSGTDFTLTISRLEPEDFGVYYCQQYGSSPGTFGQGTKVEIK

SEQ ID NO: 73 - protein, 107 aa, synthetic construct
  REGION 1..107: note = synthetic
DVVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 74 - protein, 110 aa, synthetic construct
  REGION 1..110: note = synthetic
QSALTQPPSASGSPGQSVTISCTGTSSDVGGYNHVSWYQQHPGKAPKLMIYEVSNRPSGVPNRFSGSKSGNTASLTISGLQAEDEADYYCSSYTSSNTWVFGGGTKLTVL

SEQ ID NO: 75 - protein, 112 aa, synthetic construct
  REGION 1..112: note = synthetic
EIVLTQSPLSLPVTPGEPASISCRSSQSLLHSNGNNYLNWYLQKPGQSPQLLIYLSSRRASGVPDRFSGSGSGTDFTLKISRVEAEDVGVYYCMQGLQTPWTFGQGTKVETK

SEQ ID NO: 76 - protein, 110 aa, synthetic construct
  REGION 1..110: note = synthetic
QAVLTQPPSVSGAPGQRVTISCTGSSSNIGAGYDVHWYQQLPGTAPKLLIYGNSNRPSGVPDRFSGSKSGTSAPLAITGLQAEDEADYYCQSYDSSLSTVFGGGTKLTVL

SEQ ID NO: 77 - DNA, 372 bp, synthetic construct
  misc_feature 1..372: note = synthetic
  source 1..372: mol_type = other DNA
caggttcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagattactatgatagtagtggttattactttgatgcttttgatatctggggccaagggacaatggtcaccgtctcttca

SEQ ID NO: 78 - DNA, 366 bp, synthetic construct
  misc_feature 1..366: note = synthetic
  source 1..366: mol_type = other DNA
caggtccagctggtacaatctgggggaggcgtggtccagcctgggaggtccctgagactctcctgtgcagcctctggattcaccttcagtagctatggcatccactgggtccgccaggctccaggcaaggggctggagtgggtggcaattgtatcatatgatggaagacataaatattatgcagactccgtgaagggccgattcaccatctccagagacgattccaagaacacgatctatctgcaaatggacagcctgagagctgaggacacggctgtatattactgtgcgaaagatagtgggaactccggtctggaaggtatccatgactactggggccagggaaccctggtcaccgtctcctca

SEQ ID NO: 79 - DNA, 384 bp, synthetic construct
  misc_feature 1..384: note = synthetic
  source 1..384: mol_type = other DNA
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaagtctatcatagtgggagcaccaactacaacccgtccctcaagagtcgagtcaccatatcagtagacaagtccaagaaccagttctccctgaagctgagctctgtgaccgccgcggacacggccgtgtattactgtgcgagaaacatcccccttagcagcagctggcctaattactactactactacggtatggacgtctggggccaagggaccacggtcaccgtctcctca

SEQ ID NO: 80 - DNA, 384 bp, synthetic construct
  misc_feature 1..384: note = synthetic
  source 1..384: mol_type = other DNA
caggtccagctggtgcaatctggagctgaggtgaagaagcctggggcctcagtgaaggtctcctgcaaggcttctggctacacctttaccagctatggtatcagctgggtgcgacaggcccctggacaagggcttgagtggatgggatggatcaacacttacaatggtaacacaaactatgcacagaaactccagggcagagtcaccatgaccacagacacatccacgaccacagcctacatggagctgaggagcctgagatctgacgacacggccgtatattactgtgcgagagatatcgggtatagtgggagctactattcgccctactactactacggtatggacgtctggggccaagggaccacggtcaccgtctcctca

SEQ ID NO: 81 - DNA, 390 bp, synthetic construct
  misc_feature 1..390: note = synthetic
  source 1..390: mol_type = other DNA
gaggtgcagctggtgcagtctggggctgaggtgaagaagcctggggcttcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggctgtgtattactgtgcgagagttccctcagagtattacgatttttggagtggttattataccgaagagaatgcttttgatatctggggccaagggacaatggtcaccgtctcttca

SEQ ID NO: 82 - DNA, 372 bp, synthetic construct
  misc_feature 1..372: note = synthetic
  source 1..372: mol_type = other DNA
gaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagataacggtgactacggatactactactactactacatggacgtctggggcaaagggaccacggtcaccgtctcctca

SEQ ID NO: 83 - DNA, 348 bp, synthetic construct
  misc_feature 1..348: note = synthetic
  source 1..348: mol_type = other DNA
caggtgcagctggtgcagtctggagctgaggtgaagaagcctggggcctcagtgaaggtctcctgcaaggcttctggttacaccttttccgactatggtatcagctgggtgcgacaggcccctggacaagggcttgagtggatgggatggatcagcggtgacaatgttaagactacctatgcaaagaagttccagggcagagtcaccctgaccacagacacatccacgagcacagcctacatggagctgaggagcctgacatctgacgacacggccgcatattactgtgcgagaggggggatcgtatttgactactggggccagggaaccctggtcaccgtctcctca

SEQ ID NO: 84 - DNA, 348 bp, synthetic construct
  misc_feature 1..348: note = synthetic
  source 1..348: mol_type = other DNA
caggttcagctggtgcagtctggggctgaggtgaagaagcctggggcctcagtgaaggtctcctgcaaggcttctggttacacctttaccagctatggtatcagctgggtgcgacaggccactggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaggggggatcgtatttgactactggggccagggaaccctggtcaccgtctcctca

SEQ ID NO: 85 - DNA, 357 bp, synthetic construct
  misc_feature 1..357: note = synthetic
  source 1..357: mol_type = other DNA
caaatgcagctggtacagtctggggctgaggtgaagatgcctggggcctcagtgaaggtctcctgcaaggcttctggttacacctttaccagctatggtatcagctgggtgcgacaggcccctggacaagggcttgagtggatggggtggatcagcgcttacaatggtaacacatactatgcacagaaactccagggcagagtcaccatgaccgcagacacatccacgagcacagcctacatggacctgaggagcctgagatctgacgacacggccgtgtattactgtgcgagactatcatattacaactacgctatggacgtctggggccaagggaccacggtcaccgtctcctca

SEQ ID NO: 86 - DNA, 354 bp, synthetic construct
  misc_feature 1..354: note = synthetic
  source 1..354: mol_type = other DNA
caggtacagctgcagcagtcaggggctgaggtgaagaagcctgggtcctcggtgaaggtgtcctgcaaggcttctggaggcatcttcagcacctatgtgaccagctgggtgcgacaggcccctggacaagggcttgagtggatgggagggatcatccctatgtttgacacaacagagtacgcacagaagctcgagggcagagtcacgatcaccgtggacgaatccacgaacacagcctacatggagctgagcagcctgagatttgaggacacggccgtatattactgtgcgagagcgggaggtttgagccactttgactactggggccagggaactctggtcaccgtctcgtca

SEQ ID NO: 87 - DNA, 360 bp, synthetic construct
  misc_feature 1..360: note = synthetic
  source 1..360: mol_type = other DNA
caggtccagctggtacagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacaagcacagcctacatggagctgaggagcctgagatctgacgacacggccgtgtattactgtgcgagagtgaagtgggagctagggtgggcttttgatatctggggccaagggacaatggtcaccgtctcttca

SEQ ID NO: 88 - DNA, 330 bp, synthetic construct
  misc_feature 1..330: note = synthetic
  source 1..330: mol_type = other DNA
caggctgggctgactcagccaccctcagcgtctgggacccccgggcagagggtcatcatctcttgttctggaagcagctccaacatcggaactaatactgtaagctggtatcaacacctcccaggaacggcccccaagctcctcatctataacaataatgaacggccctcaggggtccctcgccgattctctggctccaggtctggcgcctcagcctccctggccatcagtgggctccagtctgacgatgaggctcattattattgtgcagcctgggatgacaacctgactggcttggtgttcggcggagggaccaaactgaccgtccta

SEQ ID NO: 89 - DNA, 321 bp, synthetic construct
  misc_feature 1..321: note = synthetic
  source 1..321: mol_type = other DNA
gatgttgtgatgactcagtctccttcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcacactcaccatcagcagcctgcagcctgaagattttggaacttactattgtcaacaggctaacagttttccgctcactttcggcggagggaccaaggtggagatcaaa

SEQ ID NO: 90 - DNA, 321 bp, synthetic construct
  misc_feature 1..321: note = synthetic
  source 1..321: mol_type = other DNA
tcttctgagctgactcaggaccctgctgtgtctgtggccttgggacagacagtcaggatcacatgccaaggagacagcctcagaaactattatgcaagctggtaccagcagaagccaggacaggcccctgtacttgtcatctatggtaaaaacaaccggccctcagggatcccagaccgattctctggctccagctcaggaaacacagcttccttgaccatcaatggggctcaggcggaagatgaggctgactattactgtaactcccgggacagcagtgctaaacattgggtgttcggcggagggaccaagctgaccgtc

SEQ ID NO: 91 - DNA, 321 bp, synthetic construct
  misc_feature 1..321: note = synthetic
  source 1..321: mol_type = other DNA
tcttctgagctgactcaggaccctgctgtgtctgtggccttgggacagacagtcaggatcacgtgccaaggagacggcctcagaaactattttgcaagttggtaccagcagaagccaggacaggcccctgtacttgtcatctatggtaaaaacaaccggccctcagggatcccagaccgattctctggctccagctcaggaaacacagcttccttgagaatcgctggggctcaggcggaagatgaggctgactattactgtaattcccgggacggcagtggtaagcatctggtattcggcggagggaccaagttgaccgtc

SEQ ID NO: 92 - DNA, 318 bp, synthetic construct
  misc_feature 1..318: note = synthetic
  source 1..318: mol_type = other DNA
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacctggacgttcggccaagggaccaaggtggaaatcaaa

SEQ ID NO: 93 - DNA, 321 bp, synthetic construct
  misc_feature 1..321: note = synthetic
  source 1..321: mol_type = other DNA
gacatccagatgactcagtctccttccaccctgtctgcatctgtaggagacagagtcaccatcacttgccgggccagtcagagtattagtaggtggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgtcaacagagttacagtacccctcttactttcggccctgggaccaaagtggatatcaaa

SEQ ID NO: 94 - DNA, 324 bp, synthetic construct
  misc_feature 1..324: note = synthetic
  source 1..324: mol_type = other DNA
gaaacgacactcacgcagtctccaggcaccctgtctttgtctccaggggaaagagccaccctctcctgcagggccagtcagagtgttagtggcaactacttagcctggtaccagcagaaacctggccagcctcccaggctcctcatctttggtgcatccagcagggccactggcatcccagacaggttcagtggcagtgggtctgggacagacttcactctcaccatcagcagactggagcctgaagattttggagtgtattactgtcagcagtatggtagctcaccagggacgttcggccaagggaccaaggtggaaatcaaa

SEQ ID NO: 95 - DNA, 321 bp, synthetic construct
  misc_feature 1..321: note = synthetic
  source 1..321: mol_type = other DNA
gatgttgtgatgactcagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggagatcaaa

SEQ ID NO: 96 - DNA, 330 bp, synthetic construct
  misc_feature 1..330: note = synthetic
  source 1..330: mol_type = other DNA
cagtctgccctgactcagcctccctccgcgtccgggtcccctggacagtcagtcaccatctcctgcactggaaccagcagtgatgttggtggttataaccatgtctcctggtaccagcaacacccaggcaaagcccccaaactcatgatttatgaggtcagtaatcggccctcaggagttcctaatcgcttctctggctccaagtctggcaacacggcctccctgaccatctctgggctccaggctgaggacgaggctgattattactgcagctcatatacaagcagcaacacttgggtgttcggcggagggaccaagctgaccgtccta

SEQ ID NO: 97 - DNA, 336 bp, synthetic construct
  misc_feature 1..336: note = synthetic
  source 1..336: mol_type = other DNA
gaaattgtgctgactcagtctccactctccctgcccgtcacccctggagagccggcctccatctcctgcaggtctagtcagagcctcctgcatagtaatggaaacaactatttgaattggtacctgcagaagccagggcagtctccacagctcctgatctatttgagttctcgtcgggcctccggggtccctgacaggttcagtggcagtggatcaggcacagattttacactgaaaattagcagagtggaggctgaggatgttggggtttattactgcatgcaaggtctacaaactccgtggacgttcggccaagggaccaaggtggaaaccaaa

SEQ ID NO: 98 - DNA, 330 bp, synthetic construct
  misc_feature 1..330: note = synthetic
  source 1..330: mol_type = other DNA
caggcagtgctgactcagccaccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcccccctggccatcactgggctccaggctgaggacgaggctgattattactgccagtcttatgacagcagcctgagtacggtattcggcggagggaccaagctgaccgtccta

SEQ ID NO: 99 - protein, 327 aa, synthetic construct
  REGION 1..327: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 101 - protein, 106 aa, synthetic construct
  REGION 1..106: note = synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 102 - protein, 106 aa, synthetic construct
  REGION 1..106: note = synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103 - protein, 107 aa, synthetic construct [sequence omitted]

SEQ ID NO: 104 - protein, 162 aa, synthetic construct
  REGION 1..162: note = synthetic
THRTGLRSPDIPQDWVSFLRSFGQLTLCPRNGTVTGKWRGSHVVGLLTTLNFGDGPDRNKTRTFQATVLGSQMGLKGSSAGQLVLITARVTTERTAGTCLYFSAVPGILPSSQPPISCSEEGAGNATLSPRMGEECVSVWSHEGLVLTKLLTSEELALCGSR

(19) United States
(12) Patent Application Publication
(10) Pub. No.: US 2025/0270306 A1
(43) Pub. Date: Aug. 28, 2025

(54) ANTIBODIES AND USES THEREOF

(71) Applicants: OSPEDALE SAN RAFFAELE S.R.L., Milan (IT); ENTHERA S.R.L., Milan (IT)

(72) Inventors: Paolo FIORINA, Boston, MA (US); Francesca D'ADDIO, Milan (IT); Francesca ZAGARI, Milan (IT)

(21) Appl. No.: 19/011,462

(22) Filed: Jan. 6, 2025

Related U.S. Application Data
(62) Division of application No. 17/281,874, filed on Mar. 31, 2021, now Pat. No. 12,221,476.

(30) Foreign Application Priority Data
Oct. 2, 2018 (EP) 18198252.1

(51) Int. Cl. C07K 16/22; A61K 39/00

(52) U.S. Cl. CPC C07K 16/22; A61K 2039/505; C07K 2317/21; C07K 2317/24; C07K 2317/33; C07K 2317/76; C07K 2317/92

(57) ABSTRACT

The present invention relates to an antibody or antigen binding fragment thereof that binds specifically to IGFBP3 and does not displace the binding of IGF-I to IGFBP3. The antibody inhibits or reduces the binding of IGFBP3 to the TMEM219 receptor. The invention also relates to methods for their production, pharmaceutical compositions containing said antibodies, and uses thereof.

### SEQUENCE LISTING

The instant application contains a Sequence Listing which has been submitted in XML format via Patent Center and is hereby incorporated by reference in its entirety. Said XML copy, created on Jan. 2, 2025, is named eolf-othd-000001.xml and is 140,381 bytes in size.

### TECHNICAL FIELD

The present invention relates to an antibody or antigen binding fragment thereof that binds specifically to IGFBP3 and does not displace the binding of IGF-I to IGFBP3. The antibody inhibits or reduces the binding of IGFBP3 to the TMEM219 receptor. The invention also relates to methods for their production, pharmaceutical compositions containing said antibodies, and uses thereof.

### BACKGROUND ART

### IGFBP3/TMEM219 Axis

The insulin-like growth factor binding proteins is a family of seven binding proteins which modulate the bioavailability of insulin-like growth factors (IGFs). Among them IGFBP3 is the most abundant, being present in almost all tissues, and has the higher affinity for IGFs; indeed, approximately 80-90% of IGFs are bound to IGFBP3 in a ternary complex with the acid labile subunit (ALS) (1).

In addition to its ability to regulate IGFs availability, IGFBP3 has also been shown to have IGF-independent functions (2). Indeed, it is able to associate with cell-surface proteins, cell-surface receptors with integral signaling capacity, intracellular and nuclear proteins (transcription factors) thus influencing cell growth and directly inducing apoptosis (2). Among death receptors, TMEM219, a single-span membrane protein, was shown to bind specifically to IGFBP-3 but not to other IGFBP species (3). Binding of IGFBP3 to TMEM219 induces caspase-8-mediated apoptosis in a variety of cells, including cancer cells (i.e. prostate and breast) (3), but also stem cells (i.e. colonic stem cells) (4). Blocking or enhancing IGFBP3/TMEM219 axis with different strategies has been shown to respectively preventing or increasing cell death. To the best of our knowledge there are no monoclonal antibody against TMEM219 or IGFBP3 commercially available capable of preventing the IGFBP3/TMEM219 binding and halting the IGFBP3, IGF-I independent and Caspase8-mediated, detrimental effects on target tissues/cells.

### IGFBP3/TMEM219 Axis in Diabetes

Type 1 (T1D) and type 2 diabetes (T2D) are both characterized by a loss of beta cells, which results in a reduced secretion of insulin, failure to control blood glucose levels and hyperglycemia(5,6). Despite different etiological mechanisms, either autoimmune response in T1D and insulin resistance/inflammation in T2D, both lead to a progressive reduction of beta cell mass. Indeed it is becoming evident that the occurring immune activation do not appear sufficient to fully explain beta cell loss in T1D (5). Moreover, the failure of immunotherapies to cure T1D(7) highlighted that: (i) autoimmunity may not be the sole factor involved in T1D pathogenesis and (ii) alternative strategies that targets different mechanisms of disease, such as beta cell loss, are needed in order to establish an effective treatment for T1D. The observation that scattered beta cells are detected in individuals with long-standing T1D(8) confirms that either new beta cells must be occurring in order to preserve the beta cell turnover(5, 9), or the destroyed beta-cells may be “different” and prone to death(10). This may suggest that the up/down-regulated expression of surface beta cell receptors may have a key role in making them visible to immune system and, more importantly, that other non-immunological determinants may modulate beta cell fate and function. Therefore, preventing the non-immunological beta cell destruction in T1D and the progressive loss of beta cells in T2D may skew the balance between beta cell generation and destruction towards the recovery of the appropriate beta cell mass, thus paving the way for novel therapeutic approaches capable of halting or delaying the very first phase of the disease.

It has been shown that TMEM219, the IGFBP3 receptor, is expressed in a beta cell line and in human/murine islets, and that its ligation is toxic to beta cells. Interestingly, it has been also observed that mice transgenic for human IGFBP3 develop hyperglycemia, exhibit a reduced islets mass and show a decrease response to insulin-glucose stimulation (11), while those knocked down for IGFBP3 did not show any alteration in terms of glycometabolic control (12).

In humans, Drogan and colleagues recently published that elevated circulating levels of IGFBP3 are associated with the development of T2D (13). Moreover, a recent study by the Diabimmune Study group demonstrated that IGFBP3 levels correlate with autoantibody positivity and chance to seroconversion in children at risk for T1D, thus suggesting a role for circulating IGFBP3 in the early development of beta cell autoimmunity (14).

TMEM219, the IGFBP3 receptor, has been already described as a death receptor, whose activation triggers Caspase8-mediated apoptosis within the target cells thus leading to their loss(4).

### IGFBP3/TMEM219 Axis in Inflammatory Bowel Disease

Intestinal stem cells (ISCs) reside at the bottom of small and large intestine crypts and control the crypts regeneration and turnover. In particular, ISCs can differentiate along the crypts to generate goblet cells, enterocytes, enteroendocrine cells(4).

Inflammatory bowel disease (IBD) is an immune-mediated chronic condition that encompasses two clinical entities, Crohn's disease (CD) and ulcerative colitis (UC), and affects nearly 2.5 million of individuals in Europe and 1 million in USA(15). The pathogenesis of IBD is still under investigation, but recent evidences suggest that an impaired differentiation of ISCs towards Paneth cells, in ileal CD, and towards goblet cells in UC, may play a key-role in the onset of the disease. In particular, local signaling and inflammatory pathways in the mucosa both respond to external stimuli and preserve ISCs number and function, thus maintaining intestinal homeostasis (16). Indeed recently, Yancu et al., published results that support the role of IGFBP-3 in CD. Indeed, they demonstrated that, the knockout of IGFBP3 has a role in modulating inflammation in the Dextran-Sodium-Sulphate (DSS) colitis murine model(17).

The inventors have recently found that the insulin-like growth factor binding protein 3 (IGFBP3) receptor, TMEM219 receptor, is expressed on ISCs and that its interaction with the circulating hormone IGFBP3 controls ISCs fate and function in a model of intestinal disorders in diabetes, the diabetic enteropathy(4). Since diabetic enteropathy and IBD share common features, these results may add important insights in the still unknown IBD pathogenesis and will possibly lead to the introduction of a new therapeutic approach for IBD treatment.

Current available therapy for IBD is based on the use of anti-inflammatory and immunotherapeutic strategies, which are aggravated by several adverse effects and whose effectiveness in the long-term remains questionable. Surgery is also successfully employed in advanced state of the disease especially in UC (15). Relapsing of the disease mostly in CD is also frequent, thus highlighting the need for a different therapeutic approach. As a result, the identification of novel therapeutic targets and strategies in the treatment of IBD is of a high clinically relevance for the health community.

WO2016193497 and WO2016193496 describe a TMEM219 extracellular domain, ecto-TMEM that is an effective therapeutic agent. However, receptor constructs are less desirable as therapeutic agents than are antibodies. Therefore, there is still a need for antibodies that mimic the effects of ecto-TMEM.

### SUMMARY OF THE INVENTION

Disclosed herein are antibodies that bind with high affinity and specificity to human IGF binding protein 3 (IGFBP3) and that are capable of reducing or abrogating binding of IGFBP3 to its cognate receptor, TMEM219. These neutralizing antibodies are useful in treating disorders in which IGFBP3 binding to TMEM219 contributes to the pathophysiology of the disease, including diabetic enteropathy, inflammatory bowel disease (IBD), and type 1 or type 2 diabetes. Such neutralizing antibodies provide a therapeutic agent that retains the activities of the receptor-based ligand trap, ecto-TMEM219 (WO 2016/193497 and WO 2016/193496, incorporated herein by reference in their entireties), with the additional advantages of an antibody therapeutic.

In a first aspect, it is provided an isolated antibody or antigen binding fragment thereof that binds to human IGFBP3 which:
does not displace the binding of IGF-I to IGFBP3 and/or does not interfere with the binding of IGF-I to IGFBP3 and
inhibits or reduces the binding of IGFBP3 to the TMEM219 receptor.

Preferably the isolated antibody or antigen binding fragment thereof inhibits, reduces, or neutralizes the activation of the TMEM219 receptor induced by IGFBP3.

Activation of the TMEM219 receptor induced by IGFBP3 may be measured by any known method in the art or as described below. In particular, IGFBP3-induced activation of a TMEM219 receptor may be measured by measuring apoptosis increase or decrease in minigut growth as described therein.

The present invention also provides an isolated antibody or antigen binding fragment thereof that has at least one activity selected from:
a-increase in IGFBP3 treated healthy subject minigut growth
b-increase in IBD-patient minigut growth;
c-increase in diabetic enteropathy serum treated healthy subject minigut growth;
d-increase in expression of EphB2 and/or LGR5 in IGFBP3 treated healthy subject minigut;
e-decrease in caspase 8 expression in IGFBP3 treated healthy subject minigut;
f-decrease in β-cell loss in IGFBP3 treated β-cell;
g-increase in expression of insulin in IGFBP3 treated β-cell; and
h-decrease in apoptosis of β-cell in IGFBP3 treated β-cell.

Preferably the increase in a), b) and c) is by at least 20%; the increase in d) and e) is by at least 50%; the decrease in f) and the increase in g) is by at least 10%.

The invention provides an isolated antibody or antigen binding fragment thereof that binds specifically to IGFBP3, preferably free IGFBP3, and does not bind to IGFBP3 bound to IGF-I,

Free IGFBP3 is the portion of circulating IGFBP3 not complexed to IGF1 and/or IGF1/ALS.

The invention provides an isolated antibody or antigen binding fragment thereof comprising:
a. a heavy chain variable domain (VH) comprising:
   i. a CDR1 sequence of the amino acid sequence selected from the group consisting of: SEQ ID NO: 1 to SEQ ID NO: 7;
   ii. a CDR2 sequence of the amino acid sequence selected from the group consisting of: SEQ ID NO:8 to SEQ ID NO: 16; and
   iii. a CDR3 sequence of the amino acid sequence selected from the group consisting of: SEQ ID NO: 17 to SEQ ID NO:26; and
b. a light chain variable domain (VL) comprising:
   i. a CDR1 sequence of the amino acid sequence selected from the group consisting of: SEQ ID NO:27 to SEQ ID NO:36;
   ii. a CDR2 sequence of the amino acid sequence selected from the group consisting of: SEQ ID NO:37 to SEQ ID NO:44; and
   iii. a CDR3 sequence of the amino acid sequence selected from the group consisting of: SEQ ID NO:45 to SEQ ID NO:54;

Preferably the isolated antibody or antigen binding fragment thereof comprises the CDRs as indicated in Table 2 and/or in Table 3.

Preferably said antibody or antigen binding fragment thereof binds specifically to IGFBP3, and does not bind to IGFBP3 bound to IGF-I. Preferably it binds to free IGFBP3 and does not bind to IGFBP3 bound to IGF-I.

Preferably it has at least one activity selected from:
a-increase in IGFBP3 treated healthy subject minigut growth
b-increase in IBD-patient minigut growth;
c-increase in diabetic enteropathy serum treated healthy subject minigut growth;
d-increase in expression of EphB2 and/or LGR5 in IGFBP3 treated healthy subject minigut;
e-decrease in caspase 8 expression in IGFBP3 treated healthy subject minigut;
f-decrease in B-cell loss in IGFBP3 treated β-cell;
g-increase in expression of insulin in IGFBP3 treated β-cell; and
h-decrease in apoptosis of β-cell in IGFBP3 treated β-cell.

Preferably the increase in a), b) and c) is by at least 20%; the increase in d) and e) is by at least 50%; the decrease in f) and the increase in g) is by at least 10%.

Preferably the isolated antibody or antigen binding fragment thereof comprises:
a. a heavy chain variable domain sequence of the amino acid sequence selected from the group consisting of: SEQ ID NO:55 to SEQ ID NO:65;
b. a light chain variable domain sequence of the amino acid sequence selected from the group consisting of: SEQ ID NO: 66 to SEQ ID NO:76; or
c. the light chain variable domain of (a) and the heavy chain variable domain of (b).

Still preferably the isolated antibody is Yu139-A02, Yu139-A03, Yu139-B01, Yu139-C01, Yu139-C02, Yu139-D02, Yu139-D03, Yu139-F02, Yu139-G01, Yu139-G03 or Yu139-H03 or antigen binding fragment thereof, preferably Yu139-A03, Yu139-C01, Yu139-G03 or Yu139-H03 or antigen binding fragment thereof, as reported in Tables 2-7. Still preferably the isolated antibody is Yu139-A02 having SEQ ID NO:55 and SEQ ID NO: 66, Yu139-A03 having SEQ ID NO:56 and SEQ ID NO:67, Yu139-B01 having SEQ ID NO: 57 and SEQ ID NO:68, Yu139-C01 having SEQ ID NO:58 and SEQ ID NO:69, Yu139-C02 having SEQ ID NO:59 and SEQ ID NO:70, Yu139-D02 having SEQ ID NO: 60 and SEQ ID NO:71, Yu139-D03 having SEQ ID NO:61 and SEQ ID NO:72, Yu139-F02 having SEQ ID NO:62 and SEQ ID NO:73, Yu139-G01 having SEQ ID NO: 63 and SEQ ID NO:74, Yu139-G03 having SEQ ID NO:64 and SEQ ID NO:75 or Yu139-H03 having SEQ ID NO:65 and SEQ ID NO:76, preferably Yu139-A03, Yu139-C01, Yu139-G03 or Yu139-H03.

Preferably the isolated antibody or antigen binding fragment of the invention binds to human IGFBP3 with an affinity constant lower than or equal to 4×10⁻⁶ M.

The invention also provides an isolated antibody or antigen binding fragment thereof that:
(a) binds specifically to an epitope on IGFBP3, e.g., the same or similar epitope as the epitope recognized by the monoclonal antibody Yu139-A02, A03, B01, C01, C02, D02, D03, F02, G01, G03, H03 as defined in Tables 4 and 5; or
(b) cross-competes for binding with the monoclonal antibody Yu139-A02, A03, B01, C01, C02, D02, D03, F02, G01, G03, H03 as defined in Tables 4 and 5; or
(c) shows the same or similar binding affinity or specificity, or both, as any of Yu139-A02, A03, B01, C01, C02, D02, D03, F02, G01, G03, H03 as defined in Tables 4 and 5; or
(d) has one or more biological properties of an antibody molecule described herein, e.g., an antibody molecule chosen from, e.g., any of Yu139-A02, A03, B01, C01, C02, D02, D03, F02, G01, G03, H03 as defined in Tables 4 and 5; or
(e) has one or more pharmacokinetic properties of an antibody molecule described herein, e.g., an antibody molecule chosen from, e.g., any of Yu139-A02, A03, B01, C01, C02, D02, D03, F02, G01, G03, H03 as defined in Tables 4 and 5.

Preferably the isolated antibody or antigen binding fragment thereof of the invention is a human or humanized antibody.

More preferably the isolated antibody or antigen binding fragment thereof of the invention is an IgG2 or IgG4 antibody, preferably an IgG2 kappa antibody, an IgG2 lambda antibody, an IgG4 kappa antibody or an IgG4 lambda antibody.

The invention provides an isolated polynucleotide comprising at least one sequence that encodes the antibody or antigen binding fragment thereof as defined above, preferably said polynucleotide is a cDNA.

The invention provides a vector comprising the polynucleotide as defined above, preferably said vector is selected from the group consisting of a plasmid, a viral vector, a non-episomal mammalian vector, an expression vector, and a recombinant expression vector.

The invention further provides an isolated cell comprising the polynucleotide as defined above or the vector as defined above, preferably the isolated cell is a hybridoma or a Chinese Hamster Ovary (CHO) cell or a Human Embryonic Kidney cells (HEK293).

The invention further provides the antibody or antigen binding fragment thereof or the isolated polynucleotide or the vector or the isolated cell s defined above for use as a medicament, preferably for use in the treatment of: diabetes, intestinal and/or bowel disorder, malabsorption syndrome, cachexia or diabetic enteropathy, preferably diabetes is Type I or Type II diabetes preferably the intestinal and/or bowel disorder is inflammatory bowel disease, celiac disease, ulcerative colitis, Crohn's disease or intestinal obstruction.

The invention provides also a pharmaceutical composition comprising the isolated antibody or antigen binding fragment thereof or the isolated polynucleotide or the vector or the isolated cell as defined above and pharmaceutically acceptable carrier, preferably for use in the treatment of: diabetes, intestinal and/or bowel disorder, malabsorption syndrome, cachexia or diabetic enteropathy, preferably the intestinal and/or bowel disorder is inflammatory bowel disease, celiac disease, ulcerative colitis, Crohn's disease or intestinal obstruction.

The invention provides a method of inhibiting the binding of IGFBP3 to TMEM219 receptor, comprising contacting IGFBP3 with the antibody or composition as defined above.

The invention provides a method of treatment of: diabetes, preferably Type 1 or Type 2 diabetes, intestinal and/or bowel disorder, malabsorption syndrome, cachexia or diabetic enteropathy, preferably the intestinal and/or bowel disorder is inflammatory bowel disease, IBD, celiac disease, Crohn's disease or intestinal obstruction, the method comprising administering to a subject in need thereof a pharmaceutical composition comprising the isolated antibody or antigen binding fragment thereof or the isolated polynucleotide or the vector or the isolated cell as defined above and pharmaceutically acceptable carrier or administering to a subject in need thereof the isolated antibody or antigen binding fragment thereof or the isolated polynucleotide or the vector or the isolated cell as defined above.

The present invention also provides a method for producing an antibody or antigen binding fragment thereof, comprising obtaining the cell as defined above and producing the antibody or antigen binding fragment thereof.

In some embodiments, the combination includes an inhibitor of IGFBP3 (e.g., an anti-IGFBP3 antibody molecule as described herein). Thus, compositions and methods for detecting IGFBP3, as well as methods for treating various disorders including diabetes, as well as intestinal and/or bowel disorders, using the anti-IGFBP3 antibody molecules and combinations thereof are disclosed herein.

Accordingly, in one aspect, the invention features an antibody molecule (e.g., an isolated or recombinant antibody molecule) having one or more of the following properties:
(i) binds to IGFBP3, e.g., human IGFBP3, with high affinity, e.g., with an affinity constant of at least about 4×10⁶ M⁻¹, preferably 10⁷ M⁻¹, typically about 10⁸ M⁻¹ and more typically, about 10⁹ M⁻¹ to 10¹⁰ M⁻¹ or stronger;
(ii) binds to free IGFBP3 and does not substantially bind to IGFBP3 bound to IGF-I;
(iii) inhibits or reduces binding of IGFBP3 to its receptor, TMEM;
(iv) binds specifically to an epitope on IGFBP3, e.g., a different epitope from the epitope recognized by commercial antibody LSBIO LS-C45037 or clone 83.8F9;
(v) binds specifically to an epitope on IGFBP3, e.g., the same or similar epitope as the epitope recognized by the monoclonal antibody Yu139-A02, A03, B01, C01, C02, D02, D03, F02, G01, G03, H03 as defined in Tables 4 and 5;
(vi) cross-competes for binding with the monoclonal antibody Yu139-A02, A03, B01, C01, C02, D02, D03, F02, G01, G03, H03 as defined in Tables 4 and 5;
(vii) shows the same or similar binding affinity or specificity, or both, as any of Yu139-A02, A03, B01, C01, C02, D02, D03, F02, G01, G03, H03 as defined in Tables 4 and 5;
(viii) shows the same or similar binding affinity or specificity, or both, as an antibody molecule (e.g., an heavy chain variable region and light chain variable region) described in Tables 2-7;
(ix) shows the same or similar binding affinity or specificity, or both, as an antibody molecule (e.g., an heavy chain variable region and light chain variable region) having an amino acid sequence shown in Tables 2-5;
(x) shows the same or similar binding affinity or specificity, or both, as an antibody molecule (e.g., an heavy chain variable region and light chain variable region) encoded by the nucleotide sequence shown in Tables 6-7;
(xi) binds the same or an overlapping epitope with a second antibody molecule to IGFBP3, wherein the second antibody molecule is an antibody molecule described herein, e.g., an antibody molecule chosen from Yu139-A02, A03, B01, C01, C02, D02, D03, F02, G01, G03, H03 as defined in Tables 4 and 5;
(xii) has one or more biological properties of an antibody molecule described herein, e.g., an antibody molecule chosen from, e.g., any of Yu139-A02, A03, B01, C01, C02, D02, D03, F02, G01, G03, H03 as defined in Tables 4 and 5;
(xiii) has one or more pharmacokinetic properties of an antibody molecule described herein, e.g., an antibody molecule chosen from, e.g., any of Yu139-A02, A03, B01, C01, C02, D02, D03, F02, G01, G03, H03 as defined in Tables 4 and 5;
(xiv) inhibits one or more activities of IGFBP3, e.g., results in one or more of: an increase of at least 20% in the development of minigut from IBD-patient derived tissue sample when compared to untreated samples and/or an increase of at least 20% in the development of minigut growth in presence of IGFBP3 when compared to untreated samples or an increase of at least 20% in the development of minigut growth in presence of diabetic enteropathy serum when compared to untreated samples;
(xv) induces an increase in EphB2 and LGR5 of at least 50% compared to the IGFBP3-treated samples; or decrease in caspase 8 expression level of at least 50% compared to the IGFBP3-treated samples; or
(xvi) inhibits one or more activities of IGFBP3, e.g., results in one or more of: a reduction in beta cell loss, or an increase in Insulin; The reduction in beta cell loss or the increase in insulin is at least 10% compared to IGFBP3 treated samples;
(xvii) inhibits, reduces or neutralizes one or more activities of IGFBP3, resulting in blockade or reduction of IGFBP3 induced apoptosis;
(xviii) binds human IGFBP3 and is cross-reactive with cynomolgus IGFBP3.

Nucleic acid molecules encoding the antibody molecules, expression vectors, host cells and methods for making the antibody molecules are also provided. Immunoconjugates, multi- or bispecific antibody molecules and pharmaceutical compositions comprising the antibody molecules are also provided.

Without being bound to any theory, it is believed that IGFBP3/TMEM219 axis is dysfunctional in inflammatory bowel diseases (IBD) thus leading to ISCs loss and to altered function of the mucosal barrier, which is further invaded by microbes that trigger and sustain immune response activation and inflammation. The use of agents that block the IGFBP3-TMEM219 interaction in IBD may protect ISCs and preserve the integrity of the intestinal barrier, thus preventing the development of local inflammation.

Further, activation of TMEM219 signaling increases apoptosis of beta cells through upregulation of caspase 8 expression and reduced insulin expression. IGFBP3 is increased in the serum of patients with pre-T1D and pre-T2D as well as in newly diagnosed and long-standing diabetes patients and TMEM219 is expressed in beta cells. An expression or overexpression of TMEM219 favors beta cells destruction and affects beta cell mass, and the consequent hyperglycemia/inflammation perpetrates the process during diabetes onset and progression. Altered glycemic control and inflammation in pre-diabetic conditions favor an increased IGFBP3 hepatic production, which may target TMEM219 expressed on pancreatic beta cells and trigger a loop where TMEM219 overexpression parallels the increase in IGFBP3 release. Then TMEM219 may trigger beta cell death and thus targeting the IGFBP3/TMEM219 axis may prevent such cell death.

The anti-IGFBP3 antibody molecules disclosed herein can be used (alone or in combination with other agents or therapeutic modalities) to treat, prevent and/or diagnose disorders, such as diabetes, as well as intestinal and/or bowel disorders, malabsorption syndrome, inflammatory bowel disease, cachexia, IBD, celiac disease, diabetic enteropathy. Additionally, disclosed herein are methods and compositions comprising a combination of two, three or more therapeutic agents chosen from one, two, or all of the following categories (i)-(iii): (i) an agent that treat diabetes; (ii) an anti-inflammatory agent; or (iii) an immunotherapeutic agent.

The additional therapeutic agent may be selected from an agent that treat diabetes including: insulin, Insulin glargine as detailed in Vandana, 2014 (19, incorporated by reference), biguanide, glucosidase inhibitors, thiazolidinedione, DPP-4 inhibitors, GLP-1 receptor agonists as detailed in George et al 2013 (20, incorporated by reference)), an agent used to prevent diabetes, aspirin, anticoagulation and platelet anti-aggregation agents (such as enoxaparin, eparin, sulodexide); cholesterol-lowering drugs (such as statins, bile acids sequestrants, ezetimibe, fibrates as described in Marsha et al 2011 (21, incorporated by reference)); other blood pressure lowering agents (such as thiazide, ACE inhibitors, beta and alpha blockers); an anti-apoptotic agent, an anti-inflammatory agent, corticosteroids and immune suppressive agent (22, incorporated by reference), adjuvant therapy in organ transplantation, protective agent in cell therapy approach, a pain reliever, antibiotic, probiotics, TNF-alpha blockers (23, incorporated by reference), SGLT2 inhibitors (such as gliflozin derivates), integrin inhibitors (24, incorporated by reference).

Methods to measure an increase in minigut growth when compared to minigut growth in the presence of IGFBP3, and/or in the presence of diabetic entheropathy serum are known in the art and are described in several publications (4, 18, 27, 28) or as described in the method section below.

Methods to measure an increase and/or a decrease in EphB2, LGR5 or caspase 8 expression when compared to expression in the presence of IGFBP3 are known in the art and include quantitative RT-PCR, Realt-Time RT-PCR, microarray, northern blotting, RNA-Seq (29,30) or as described in the method section below.

Methods to measure a decrease in beta-cell loss when compared to beta-cell loss in the presence of IGFBP3 are known in the art and include cell proliferation assays (CFSE staining, Calcein/PI staining, Trypan Blue exclusion, BrdU staining, MTT) apoptosis assays (TUNEL, Caspase activation and detection, Annexin V binding) or as described in the method section below.

Methods to measure an increase in insulin level when compared to insulin level in the presence of IGFBP3 are known in the art and include western blots, ELISA mass spectrometry (31-33).

Methods to measure a decrease in apoptosis when compared to apoptosis in the presence of IGFBP3 are known in the art and include DNA fragmentation, Caspase activation analysis, Mithocondrial membrane permeabilization, Annexin V binding (34) or as described in the method section below.

In some embodiments, the antibody molecule binds to IGFBP3 with high affinity, e.g., with a KD that is about the same, or at least about 10%, 20%, 30%, 40%, 50%, 60%, 70%, 80% or 90% higher or lower than the KD of a murine anti-IGFBP3 antibody molecule or chimeric anti-IGFBP3 antibody molecule or a commercial anti-IGFBP3 antibody molecule. In some embodiments, the KD of the murine or chimeric anti-IGFBP3 antibody molecule is less than about 0.4, 0.3, 0.2, 0.1, or 0.05 nM, e.g., measured by a Biacore method or KinExA=kinetic exclusion assays. In some embodiments, the KD of the murine or chimeric anti-IGFBP3 antibody molecule is less than about 0.2 nM. In other embodiments, the KD of the murine or chimeric anti IGFBP3 antibody molecule is less than about 10, 5, 3, 2, or 1 nM, e.g., measured by binding on cells expressing IGFBP3 (e.g., 300.19 cells). In some embodiments, the KD of the murine or chimeric anti IGFBP3 antibody molecule is less than about 1 nM.

Methods to measure binding to IGFBP3 are known in the art as protein-protein interactions assays and include ELISA, co-immunoprecipitation, surface plasmon resonance, FRET-Forster resonance energy transfer (35) or as described in the method section below.

In some embodiments, the expression level of the antibody molecule is higher, e.g., at least about 0.5, 1, 2, 3, 4, 5, 6, 7, 8, 9 or 10-fold higher, than the expression level of a murine or chimeric antibody molecule, e.g., a murine, commercial or chimeric anti-IGFBP3 antibody molecule such as LSBIO LS-C45037, clone 83.8F9 or Novus NBP2-12364. In some embodiments, the antibody molecule is expressed in HEK293 cells, CHO cells or any suitable mammalian cell line known in the art.

In some embodiments, the anti-IGFBP3 antibody molecule reduces one or more IGFBP3-associated activities with an IC50 (concentration at 50% inhibition) that is about the same or lower, e.g., at least about 10%, 20%, 30%, 40%, 50%, 60%, 70%, 80% or 90% lower, than the IC50 of a murine, commercial or chimeric anti-IGFBP3 antibody molecule, e.g., a murine commercial or chimeric anti-IGFBP3 antibody molecule described herein.

In some embodiments, the anti-IGFBP3 antibody molecule has improved stability, e.g., at least about 0.5, 1, 2, 3, 4, 5, 6, 7, 8, 9 or 10-fold more stable in vivo or in vitro, than a murine, commercial or chimeric anti-IGFBP3 antibody molecule, e.g., a murine, commercial or chimeric anti-IGFBP3 antibody molecule such as LSBIO LS-C45037, clone 83.8F9 or Novus NBP2-12364.

In one embodiment, the anti IGFBP3 antibody molecule is a humanized antibody molecule.

In another embodiment, the anti-IGFBP3 antibody molecule comprises at least one antigen-binding region, e.g., a variable region or an antigen-binding fragment thereof, from an antibody described herein, e.g., an antibody chosen from any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences.

In yet another embodiment, the anti-IGFBP3 antibody molecule comprises at least one, two, three or four variable regions from an antibody described herein, e.g., an antibody chosen from any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences.

In yet another embodiment, the anti-IGFBP3 antibody molecule comprises at least one or two heavy chain variable regions from an antibody described herein, e.g., an antibody chosen from any of Yu139-A02, A03, B01, C01, C02, D02, D03, F02, G01, G03, H03;

or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences.

In yet another embodiment, the anti-IGFBP3 antibody molecule comprises at least one or two light chain variable regions from an antibody described herein, e.g., an antibody chosen from any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences.

In yet another embodiment, the anti-IGFBP3 antibody molecule includes a heavy chain constant region for an IgG4, e.g., a human IgG4. In one embodiment, the human IgG4 includes a substitution at position 228 (e.g., a Ser to Pro substitution). In still another embodiment, the anti-IGFBP3 antibody molecule includes a heavy chain constant region for an IgG1, e.g., a human IgG1. In one embodiment, the human IgG1 includes a substitution at position 297 (e.g., an Asn to Ala substitution). In one embodiment the human IgG1 includes a substitution at position 250, a substitution at position 428, or both (e.g., a Thr to Gln substitution at position 250 and/or a Met to Leu substitution at position 428). In one embodiment, the human IgG1 includes a substitution at position 234, a substitution at position 235, or both (e.g., a Leu to Ala substitution at position 234 and/or a Leu to Ala substitution at position 235). In one embodiment, the heavy chain constant region comprises an amino sequence set forth in Table 8, or a sequence substantially identical (e.g., at least 80%, 85%, 90%, 92%, 95%, 97%, 98%, 99% or higher identical) thereto.

In yet another embodiment, the anti-IGFBP3 antibody molecule includes a kappa light chain constant region, e.g., a human kappa light chain constant region. In one embodiment, the light chain constant region comprises an amino sequence set forth in Table 8, or a sequence substantially identical (e.g., at least 80%, 85%, 90%, 92%, 95%, 97%, 98%, 99% or higher identical) thereto.

In another embodiment, the anti-IGFBP3 antibody molecule includes a heavy chain constant region for an IgG4, e.g., a human IgG4, and a kappa light chain constant region, e.g., a human kappa light chain constant region, e.g., a heavy and light chain constant region comprising an amino sequence set forth in Table 8, or a sequence substantially identical (e.g., at least 80%, 85%, 90%, 92%, 95%, 97%, 98%, 99% or higher identical) thereto. In yet another embodiment, the anti-IGFBP3 antibody molecule includes a heavy chain constant region for an IgG1, e.g., a human IgG1, and a kappa light chain constant region, e.g., a human kappa light chain constant region, e.g., a heavy and light chain constant region comprising an amino sequence set forth in Table 8, or a sequence substantially identical (e.g., at least 80%, 85%, 90%, 92%, 95%, 97%, 98%, 99% or higher identical) thereto. In one embodiment, the human IgG1 or IgG4 includes a substitution at the variable region to decrease aggregation, reduce charge heterogeneity, increase affinity and modulate antigen binding; removal by mutation of instability hotspot in the CDR, putative N-glycosylation sites in the variable region as described in (26), incorporated by reference.

In another embodiment, the anti-IGFBP3 antibody molecule includes a heavy chain variable domain and a constant region, a light chain variable domain and a constant region, or both, comprising the amino acid sequence of any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences. The anti-IGFBP3 antibody molecule, optionally, comprises a leader sequence from a heavy chain, a light chain, or both.

In yet another embodiment, the anti-IGFBP3 antibody molecule includes at least one, two, or three complementarity determining regions (CDRs) from a heavy chain variable region of an antibody described herein, e.g., an antibody chosen from any of any of Yu139-A02, A03, B01, C01, C02, D02, D03, F02, G01, G03, H03, or BAP058-Clone-O; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences.

In yet another embodiment, the anti-IGFBP3 antibody molecule includes at least one, two, or three CDRs (or collectively all of the CDRs) from a heavy chain variable region comprising an amino acid sequence shown in Tables 2-5 or encoded by a nucleotide sequence shown in Tables 6-7. In one embodiment, one or more of the CDRs (or collectively all of the CDRs) have one, two, three, four, five, six or more changes, e.g., amino acid substitutions or deletions, relative to the amino acid sequence shown in Tables 2-5, or encoded by a nucleotide sequence shown in Tables 6-7.

In yet another embodiment, the anti-IGFBP3 antibody molecule includes at least one, two, or three CDRs from a light chain variable region of an antibody described herein, e.g., an antibody chosen from any of any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequence. In yet another embodiment, the anti-IGFBP3 antibody molecule includes at least one, two, or three CDRs (or collectively all of the CDRs) from a light chain variable region comprising an amino acid sequence shown in Tables 2-5 or encoded by a nucleotide sequence shown in Tables 6-7. In one embodiment, one or more of the CDRs (or collectively all of the CDRs) have one, two, three, four, five, six or more changes, e.g., amino acid substitutions or deletions, relative to the amino acid sequence shown in Tables 2-5, or encoded by a nucleotide sequence shown in Tables 6-7. In certain embodiments, the anti-IGFBP3 antibody molecule includes a substitution in a light chain CDR, e.g., one or more substitutions in a CDR1, CDR2 and/or CDR3 of the light chain. In another embodiment, the anti-IGFBP3 antibody molecule includes at least one, two, three, four, five or six CDRs (or collectively all of the CDRs) from a heavy and light chain variable region comprising an amino acid sequence shown in Tables 2-5, or encoded by a nucleotide sequence shown in Tables 6-7. In one embodiment, one or more of the CDRs (or collectively all of the CDRs) have one, two, three, four, five, six or more changes, e.g., amino acid substitutions or deletions, relative to the amino acid sequence shown in Tables 2-5, or encoded by a nucleotide sequence shown in Tables 6-7.

In one embodiment, the anti-IGFBP3 antibody molecule includes all six CDRs from an antibody described herein, e.g., an antibody chosen from any of any of Yu139-A02, A03, B01, C01, C02, D02, D03, F02, G01, G03, H03; or as described in Tables 2-5, or encoded by the nucleotide sequence in Tables 6-7, or closely related CDRs, e.g., CDRs which are identical or which have at least one amino acid alteration, but not more than two, three or four alterations (e.g., substitutions, deletions, or insertions, e.g., conservative substitutions). In one embodiment, the anti-IGFBP3 antibody molecule may include any CDR described herein. In certain embodiments, the anti-IGFBP3 antibody molecule includes a substitution in a light chain CDR, e.g., one or more substitutions in a CDR1, CDR2 and/or CDR3 of the light chain. In another embodiment, the anti-IGFBP3 antibody molecule includes at least one, two, or three CDRs according to Kabat et al. (e.g., at least one, two, or three CDRs according to the Kabat definition as set out in Tables 2-5) from a heavy chain variable region of an antibody described herein, e.g., an antibody chosen from any of any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Tables 2-3.

In another embodiment, the anti-IGFBP3 antibody molecule includes at least one, two, or three CDRs according to Kabat et al. (e.g., at least one, two, or three CDRs according to the Kabat definition as set out in Tables 2-3) from a light chain variable region of an antibody described herein, e.g., an antibody chosen from any of any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Tables 2-3.

In yet another embodiment, the anti-IGFBP3 antibody molecule includes at least one, two, three, four, five, or six CDRs according to Kabat et al. (e.g., at least one, two, three, four, five, or six CDRs according to the Kabat definition as set out in Tables 2-3) from the heavy and light chain variable regions of an antibody described herein, e.g., an antibody chosen from any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Tables 2-3. In yet another embodiment, the anti-IGFBP3 antibody molecule includes all six CDRs according to Kabat et al. (e.g., all six CDRs according to the Kabat definition as set out in Tables 2-3) from the heavy and light chain variable regions of an antibody described herein, e.g., an antibody chosen from any of any of Yu139-A02, A03, B01, C01, C02, D02, D03, F02, G01, G03, H03; or as described in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Tables 2-3. In one embodiment, the anti-IGFBP3 antibody molecule may include any CDR described herein.

In another embodiment, the anti-IGFBP3 antibody molecule includes at least one, two, or three Chothia or Kabat hypervariable loops (e.g., at least one, two, or three hypervariable loops according to the Chothia or Kabat definition as set out in Tables 2-5) from a heavy chain variable region of an antibody described herein, e.g., an antibody chosen from any of any of Yu139-A02, A03, B01, C01, C02, D02, D03, F02, G01, G03, H03; or as described in Tables 2-5, or encoded by the nucleotide sequence in Tables 6-7; or at least the amino acids from those hypervariable loops that contact IGFBP3; or which have at least one amino acid alteration, but not more than two, three or four alterations (e.g., substitutions, deletions, or insertions, e.g., conservative substitutions) relative to one, two, or three hypervariable loops according to Chothia et al. shown in Tables 2-5.

In another embodiment, the anti-IGFBP3 antibody molecule includes at least one, two, or three Chothia hypervariable loops (e.g., at least one, two, or three hypervariable loops according to the Chothia definition as set out in Tables 2-5) of a light chain variable region of an antibody described herein, e.g., an antibody chosen from any of any of Yu139-A02, A03, B01, C01, C02, D02, D03, F02, G01, G03, H03; or as described in Tables 2-5, or encoded by the nucleotide sequence in Tables 6-7; or at least the amino acids from those hypervariable loops that contact IGFBP3; or which have at least one amino acid alteration, but not more than two, three or four alterations (e.g., substitutions, deletions, or insertions, e.g., conservative substitutions) relative to one, two, or three hypervariable loops according to Chothia et al. shown in Tables 2-5.

In yet another embodiment, the anti-IGFBP3 antibody molecule includes at least one, two, three, four, five, or six hypervariable loops (e.g., at least one, two, three, four, five, or six hypervariable loops according to the Chothia definition as set out in Tables 2-5) from the heavy and light chain variable regions of an antibody described herein, e.g., an antibody chosen from any of any of Yu139-A02, A03, B01, C01, C02, D02, D03, F02, G01, G03, H03; or as described in Tables 2-5, or encoded by the nucleotide sequence in Tables 6-7; or at least the amino acids from those hypervariable loops that contact IGFBP3; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Tables 2-5.

In one embodiment, the anti-IGFBP3 antibody molecule includes all six hypervariable loops (e.g., all six hypervariable loops according to the Chothia definition as set out in Tables 2-5) of an antibody described herein, e.g., an antibody chosen from any of any of Yu139-A02, A03, B01, C01, C02, D02, D03, F02, G01, G03, H03, or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Tables 2-5. In one embodiment, the anti-IGFBP3 antibody molecule may include any hypervariable loop described herein.

In still another embodiment, the anti-IGFBP3 antibody molecule includes at least one, two, or three hypervariable loops that have the same canonical structures as the corresponding hypervariable loop of an antibody described herein, e.g., an antibody chosen from any of any of Yu139-A02, A03, B01, C01, C02, D02, D03, F02, G01, G03, H03, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In certain embodiments, the anti-IGFBP3 antibody molecule includes a combination of CDRs or hypervariable loops defined according to the Kabat et al. and Chothia et al.

In one embodiment, the anti-IGFBP3 antibody molecule includes at least one, two or three CDRs or hypervariable loops from a heavy chain variable region of an antibody described herein, e.g., an antibody chosen from any of any of Yu139-A02, A03, B01, C01, C02, D02, D03, F02, G01, G03, H03, according to the Kabat and Chothia definition (e.g., at least one, two, or three CDRs or hypervariable loops according to the Kabat and Chothia definition as set out in Tables 2-5); or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Tables 2-5.

For example, the anti-IGFBP3 antibody molecule can include VH CDR1 according to Kabat et al. or VH hypervariable loop 1 according to Chothia et al., or a combination thereof, e.g., as shown in Tables 2-5. The anti-IGFBP3 antibody molecule can further include, e.g., VH CDRs 2-3 according to Kabat et al. and VL CDRs 1-3 according to Kabat et al., e.g., as shown in Tables 2-5. Accordingly, in some embodiments, framework regions are defined based on a combination of CDRs defined according to Kabat et al. and hypervariable loops defined according to Chothia et al. For example, the anti-IGFBP3 antibody molecule can include VH FR1 defined based on VH hypervariable loop 1 according to Chothia et al. and VH FR2 defined based on VH CDRs 1-2 according to Kabat et al., e.g., as shown in Tables 2-5. The anti-IGFBP3 antibody molecule can further include, e.g., VH FRs 3-4 defined based on VH CDRs 2-3 according to Kabat et al. and VL FRs 1-4 defined based on VL CDRs 1-3 according to Kabat et al. The anti-IGFBP3 antibody molecule can contain any combination of CDRs or hypervariable loops according to the Kabat and Chothia definitions. In one embodiment, the anti-IGFBP3 antibody molecule includes at least one, two or three CDRs from a light chain variable region of an antibody described herein, e.g., an antibody chosen from any of any of Yu139-A02, Yu139-A03, Yu139-B01, Yu139-C01, Yu139-C02, Yu139-D02, Yu139-D03, Yu139-F02, Yu139-G01, Yu139-G03, Yu139-H03, according to the Kabat and Chothia definition (e.g., at least one, two, or three CDRs according to the Kabat and Chothia definition as set out in Tables 2-5). Preferred anti-IGFBP3 antibodies are Yu139-A03, Yu139-C01, Yu139-H03 or Yu139-G03.

In an embodiment, e.g., an embodiment comprising a variable region, a CDR (e.g., Chothia CDR or Kabat CDR), or other sequence referred to herein, e.g., in Tables 2-5, the antibody molecule is a monospecific antibody molecule, a bispecific antibody molecule, or is an antibody molecule that comprises an antigen binding fragment of an antibody, e.g., a half antibody or antigen binding fragment of a half antibody. In embodiments the antibody molecule is a bispecific antibody molecule having a first binding specificity for IGFBP3 and a second binding specificity for TNF-alpha, integrin, IL1, IL12 and IL23, CD3, CD20, CD80, CD86.

In one embodiment, the anti-IGFBP3 antibody molecule includes:
(i) a heavy chain variable region (VH) including a VHCDR1 amino acid sequence chosen from any one of SEQ ID NO: 1 to SEQ ID NO: 7; a VHCDR2 amino acid sequence chosen from any one of SEQ ID NO: 8 to SEQ ID NO: 16; and a VHCDR3 amino acid sequence chosen from any one of SEQ ID NO: 17 to SEQ ID NO: 26; and
(ii) a light chain variable region (VL) including a VLCDR1 amino acid sequence chosen from any one of SEQ ID NO: 27 to SEQ ID NO: 36, a VLCDR2 amino acid sequence chosen from any one of SEQ ID NO: 37 to SEQ ID NO: 44, and a VLCDR3 amino acid sequence chosen from SEQ ID NO: 45 to SEQ ID NO: 54.

In another embodiment, the anti-IGFBP3 antibody molecule includes:
(i) a heavy chain variable region (VH) including a VHCDR1 amino acid sequence chosen from SEQ ID NO: 2, SEQ ID NO: 4 or SEQ ID NO: 7; a VHCDR2 amino acid sequence of SEQ ID NO: 9, SEQ ID NO: 11, SEQ ID NO: 12 or SEQ ID NO: 16 and a VHCDR3 amino acid sequence of SEQ ID NO: 18, SEQ ID NO: 20, SEQ ID NO: 25 or SEQ ID NO: 26 and
(ii) a light chain variable region (VL) including a VLCDR1 amino acid sequence of SEQ ID NO: 28, SEQ ID NO: 30, SEQ ID NO: 35, SEQ ID NO: 36, a VLCDR2 amino acid sequence of SEQ ID NO: 38, SEQ ID NO: 39, SEQ ID NO: 43, SEQ ID NO: 44 and a VLCDR3 amino acid sequence of SEQ ID NO: 46, SEQ ID NO: 48, SEQ ID NO: 53, SEQ ID NO: 54.

In one embodiment, the light or the heavy chain variable framework (e.g., the region encompassing at least FR1, FR2, FR3, and optionally FR4) of the anti-IGFBP3 antibody molecule can be chosen from: (a) a light or heavy chain variable framework including at least 80%, 85%, 87% 90%, 92%, 93%, 95%, 97%, 98%, or preferably 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In certain embodiments, the anti-IGFBP3 antibody molecule comprises a heavy chain variable domain having at least one, two, three, four, five, six, seven, ten, fifteen, twenty or more changes, e.g., amino acid substitutions or deletions.

In one embodiment, the heavy or light chain variable region, or both, of the anti-IGFBP3 antibody molecule includes an amino acid sequence encoded by a nucleic acid sequence described herein or a nucleic acid that hybridizes to a nucleic acid sequence described herein (e.g., a nucleic acid sequence as shown in Tables 6 and 7) or its complement, e.g., under low stringency, medium stringency, or high stringency, or other hybridization condition described herein.

In another embodiment, the anti-IGFBP3 antibody molecule comprises at least one, two, three, or four antigen-binding regions, e.g., variable regions, having an amino acid sequence as set forth in Tables 2-5, or a sequence substantially identical thereto (e.g., a sequence at least about 85%, 90%, 95%, 99% or more identical thereto, or which differs by no more than 1, 2, 5, 10, or 15 amino acid residues from the sequences shown in Tables 2-5. In another embodiment, the anti-IGFBP3 antibody molecule includes a VH and/or VL domain encoded by a nucleic acid having a nucleotide sequence as set forth in Tables 2-5, or a sequence substantially identical thereto (e.g., a sequence at least about 85%, 90%, 95%, 99% or more identical thereto, or which differs by no more than 3, 6, 15, 30, or 45 nucleotides from the sequences shown in Tables 2-5.

In yet another embodiment, the anti-IGFBP3 antibody molecule comprises at least one, two, or three CDRs from a heavy chain variable region having an amino acid sequence as set forth in Tables 2-5, or a sequence substantially homologous thereto (e.g., a sequence at least about 85%, 90%, 95%, 99% or more identical thereto, and/or having one, two, three or more substitutions, insertions or deletions, e.g., conserved substitutions). In yet another embodiment, the anti-IGFBP3 antibody molecule comprises at least one, two, or three CDRs from a light chain variable region having an amino acid sequence as set forth in Tables 2-5, or a sequence substantially homologous thereto (e.g., a sequence at least about 85%, 90%, 95%, 99% or more identical thereto, and/or having one, two, three or more substitutions, insertions or deletions, e.g., conserved substitutions). In yet another embodiment, the anti-IGFBP3 antibody molecule comprises at least one, two, three, four, five or six CDRs from heavy and light chain variable regions having an amino acid sequence as set forth in Tables 2-5), or a sequence substantially homologous thereto (e.g., a sequence at least about 85%, 90%, 95%, 99% or more identical thereto, and/or having one, two, three or more substitutions, insertions or deletions, e.g., conserved substitutions).

In yet other embodiments, the anti-IGFBP3 antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG1 or IgG4 (e.g., human IgG1, IgG2 or IgG4). In one embodiment, the heavy chain constant region is human IgG1. In another embodiment, the anti-IGFBP3 antibody molecule has a light chain constant region chosen from, e.g., the light chain constant regions of kappa or lambda. In one embodiment, the constant region is altered, e.g., mutated, to modify the properties of the anti-IGFBP3 antibody molecule (e.g., to increase or decrease one or more of: Fc receptor binding, antibody glycosylation, the number of cysteine residues, effector cell function, complement function, half-life, aggregation and stability). In certain embodiments, the anti-IGFBP3 antibody molecules comprises a human IgG4 mutated

In one embodiment, the anti-IGFBP3 antibody molecule is isolated or recombinant.

In one embodiment, the anti-IGFBP3 antibody molecule is a humanized or human antibody molecule.

The invention also features a nucleic acid molecule that comprise one or both nucleotide sequences that encode heavy and light chain variable regions, CDRs, hypervariable loops, framework regions of the anti-IGFBP3 antibody molecules, as described herein. In certain embodiments, the nucleotide sequence that encodes the anti-IGFBP3 antibody molecule is codon optimized. For example, the invention features a first and second nucleic acid encoding heavy and light chain variable regions, respectively, of an anti-IGFBP3 antibody molecule chosen from one or more of, e.g., any of Yu139-A02, A03, B01, C01, C02, D02, D03, F02, G01, G03, H03, as summarized in Tables 2-5, or a sequence substantially identical thereto. For example, the nucleic acid can comprise a nucleotide sequence as set forth in Tables 6-7, or a sequence substantially identical thereto (e.g., a sequence at least about 85%, 90%, 95%, 99% or more identical thereto, or which differs by no more than 3, 6, 15, 30, or 45 nucleotides from the sequences shown in Tables 6-7).

In other embodiments, the nucleic acid molecule comprises a nucleotide sequence that encodes a heavy chain variable domain and/or a heavy chain constant region comprising the amino acid sequence of any of Yu139-A02, A03, B01, C01, C02, D02, D03, F02, G01, G03, H03; or as described in Tables 2-5; or the nucleotide sequence in Tables 6-7; or a sequence substantially identical (e.g., a sequence at least about 85%, 90%, 95%, 99% or more identical) to any of the aforesaid sequences.

In other embodiments, the nucleic acid molecule comprises a nucleotide sequence that encodes a light chain variable domain and/or a light chain constant region comprising the amino acid sequence of any of Yu139-A02, A03, B01, C01, C02, D02, D03, F02, G01, G03, H03; or as described in Tables 2-5; or the nucleotide sequence in Tables 6-7; or a sequence substantially identical (e.g., a sequence at least about 85%, 90%, 95%, 99% or more identical) to any of the aforesaid sequences.

The aforesaid nucleotide sequences encoding the anti-IGFBP3 heavy and light chain variable domain and constant regions can be present in a separate nucleic acid molecule, or in the same nucleic acid molecule. In certain embodiments, the nucleic acid molecules comprise a nucleotide sequence encoding a leader sequence.

In certain embodiments, the nucleic acid molecule comprises a nucleotide sequence encoding at least one, two, or three CDRs, or hypervariable loops, from a heavy chain variable region having an amino acid sequence as set forth in Tables 2-5, or a sequence substantially homologous thereto (e.g., a sequence at least about 85%, 90%, 95%, 99% or more identical thereto, and/or having one, two, three or more substitutions, insertions or deletions, e.g., conserved substitutions).

In another embodiment, the nucleic acid molecule comprises a nucleotide sequence encoding at least one, two, or three CDRs, or hypervariable loops, from a light chain variable region having an amino acid sequence as set forth in Tables 2-5, or a sequence substantially homologous thereto (e.g., a sequence at least about 85%, 90%, 95%, 99% or more identical thereto, and/or having one, two, three or more substitutions, insertions or deletions, e.g., conserved substitutions).

In yet another embodiment, the nucleic acid molecule comprises a nucleotide sequence encoding at least one, two, three, four, five, or six CDRs, or hypervariable loops, from heavy and light chain variable regions having an amino acid sequence as set forth in Tables 2-5, or a sequence substantially homologous thereto (e.g., a sequence at least about 85%, 90%, 95%, 99% or more identical thereto, and/or having one, two, three or more substitutions, insertions or deletions, e.g., conserved substitutions).

In another embodiment, the nucleic acid molecule includes one or more heavy chain framework region (e.g., any of VHFW1 (type a), VHFW1 (type b), VHFW1 (type c), VHFW1 (type d), VHFW2 (type a), VHFW2 (type a′), VHFW2 (type b), VHFW2 (type c), VHFW2 (type d), VHFW2 (type e), VHFW3 (type a), VHFW3 (type b), VHFW3 (type c), VHFW3 (type d), VHFW3 (type e), or VHFW4, or any combination thereof, e.g., a framework combination as described herein) for any of Yu139-A02, A03, B01, C01, C02, D02, D03, F02, G01, G03, H03, as summarized in Tables 2-5, or a sequence substantially identical thereto. For example, the nucleic acid molecule can comprise a nucleotide sequence as set forth in Tables 6-7, or a sequence substantially identical thereto (e.g., a sequence at least about 85%, 90%, 95%, 99% or more identical thereto, or which differs by no more than 3, 6, 15, 30, or 45 nucleotides from the sequences shown in Tables 6-7).

In another embodiment, the nucleic acid molecule includes one or more light chain framework region (e.g., any of VLFW1 (type a), VLFW1 (type b), VLFW1 (type c), VLFW1 (type d), VLFW1 (type e), VLFW1 (type f), VLFW2 (type a), VLFW2 (type c), VLFW3 (type a), VLFW3 (type b), VLFW3 (type c), VLFW3 (type d), VLFW3 (type e), VLFW3 (type f), VLFW3 (type g), or VLFW4, or any combination thereof, e.g., a framework combination as described herein) for of any of Yu139-A02, A03, B01, C01, C02, D02, D03, F02, G01, G03, H03, as summarized in Tables 2-5, or a sequence substantially identical thereto. For example, the nucleic acid molecule can comprise a nucleotide sequence as set forth in Tables 6-7, or a sequence substantially identical thereto (e.g., a sequence at least about 85%, 90%, 95%, 99% or more identical thereto, or which differs by no more than 3, 6, 15, 30, or 45 nucleotides from the sequences shown in Tables 6-7).

In another embodiment, the nucleic acid molecule includes one or more heavy chain framework region and one or more light chain framework region as described herein.

The heavy and light chain framework regions may be present in the same vector or separate vectors.

In another aspect, the application features host cells and vectors containing the nucleic acids described herein or modified for codon optimization according to known methods. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In one aspect, the invention features a method of providing an antibody molecule described herein. The method includes: providing a IGFBP3 antigen (e.g., an antigen comprising at least a portion of a IGFBP3 epitope); obtaining an antibody molecule that specifically binds to the IGFBP3 polypeptide; and evaluating if the antibody molecule specifically binds to the IGFBP3 polypeptide, or evaluating efficacy of the antibody molecule in modulating, e.g., inhibiting, the activity of the IGFBP3. The method can further include administering the antibody molecule to a subject, e.g., a human or non-human animal.

In another aspect, the invention provides, compositions, e.g., pharmaceutical compositions, which include a pharmaceutically acceptable carrier, excipient or stabilizer, and at least one of the anti-IGFBP3 antibody molecules described herein. In one embodiment, the composition, e.g., the pharmaceutical composition, includes a combination of the antibody molecule and one or more agents, e.g., a therapeutic agent or other antibody molecule, as described herein. In one embodiment, the antibody molecule is conjugated to a label or a therapeutic agent.

The anti-IGFBP3 antibody molecules disclosed herein can inhibit, reduce or neutralize one or more activities of IGFBP3 as indicated above. Thus, such antibody molecules can be used to treat or prevent disorders where the inhibition, reduction or neutralization of IGFBP3-induced activities in a subject is desired.

### Uses of the Anti-IGFBP3 Antibody Molecules

The present antibodies are used in methods of treatment of various disorders or conditions such as diabetes, as well as intestinal bowel diseases, malabsorption syndrome, inflammatory bowel disease, cachexia, Crohn's disease, ulcerative colitis, celiac disease, diabetic enteropathy.

Accordingly, in another aspect, a method of modulating the IGFBP3/TMEM219 axis in a subject is provided. The method comprises administering to the subject an anti-IGFBP3 antibody molecule disclosed herein (e.g., a therapeutically effective amount of an anti-IGFBP3 antibody molecule), alone or in combination with one or more agents or procedures, such that the IGFBP3/TMEM219 axis in the subject is modulated. In one embodiment, the antibody molecule inhibits, reduce or neutralize or block the IGFBP3/TMEM219 axis activity in the subject. The subject can be a mammal, e.g., a primate, preferably a higher primate, e.g., a human (e.g., a patient having, or at risk of having, a disorder described herein). In one embodiment, the subject is in need of inhibiting, reducing, neutralizing or blocking the IGFBP3/TMEM219 axis. In one embodiment, the subject has, or is at risk of, having a disorder described herein, e.g., diabetes, or inflammatory bowel disorder (IBD), malabsorption syndrome, irritable bowel disease, cachexia, celiac disease, diabetic enteropathy as described herein.

### BRIEF DESCRIPTION OF THE DRAWINGS

FIGS. 1A-1B. Commercial monoclonal antibody anti-IGFBP3 (LSBIO LSC45037 clone 83.8F9) binds IGFBP3 but does not inhibit the interaction with the extracellular domain of TMEM219. FIG. 1A provides a schematic of the assay. In this ELISA, the plate is coated with LSBIO anti-IGFBP3 monoclonal antibody (capture antibody). Then a premix of IGFBP3 and ecto-TMEM219 is added. IGFBP3 in the IGFBP3/ecto-TMEM219 complex should bind to the commercial LSBio antibody on the plate. If the capture antibody competes with ecto-TMEM219, the ecto-TMEM219 should be displaced from the complex and removed with washing. Detection of bound TMEM219 is performed with an anti-His tag reagent (ecto-TMEM219 has the His tag); if ecto-TMEM219 is displaced, absorbance is low. FIG. 1B shows the results. The leftmost three bars show various controls. In each case the commercial LSBio anti-IGFBP3 is coated on the plate. In the first control experiment (leftmost bar), IGFBP3/ecto-TMEM219 mix is omitted to control for nonspecific binding of the anti-His tag to the plate or the commercial anti-IGFBP3 antibody coated on the plate. In the second control experiment, only IGFBP3 is omitted to control for nonspecific binding of ecto-TMEM219 to the plate or to the LSBio anti-IGFBP3 capture antibody. In the third control, ecto-TMEM219 is omitted, to control for nonspecific binding of the anti-His tag to IGFBP3. The rightmost bar shows absorbance after addition of IGFBP3: TMEM-219 (1:1) complex. The high absorbance indicates that the LSBio antibody does not compete with ecto-TMEM219 for binding to IGFBP3.

FIG. 2. Effect of commercially available anti-IGFBP3 antibodies, LSBIO LS-C149975/72664 clone 83.8F9) in rescuing mini-guts development in a model of diabetic enteropathy (DE, n=3). Monoclonal antibody was tested at a 1:10 and 1:50 ratio compared to ecto-TMEM219, while polyclonal antibody was tested at a 1:10 ratio, only. ****p<0.0001 Medium DE vs. Medium Healthy, Ecto-TMEM219 vs. Medium DE.

FIG. 3. Effect of commercially available anti-IGFBP3 antibodies (LSBIO LS-C45037/clone 83.8F9) in rescuing mini-guts development in the model of Crohn's disease (IBD, n=3). Monoclonal antibody was tested at a 1:1, 1:10 and 1:50 ratio compared to ecto-TMEM219, while polyclonal antibody was tested at a 1:10 ratio, only. ****p<0.0001 IBD patient vs. Healthy CTRL; *** p<0.001 Ecto-TMEM219 vs. IBD patient.

FIG. 4A-4C. ISCs (intestinal stem cells) markers expression (4A) EphB2, (4B) LGR5 and (4C) h-TERT analyzed by RT-PCR in mini-guts obtained from Crohn's disease patients (IBD) treated with anti-IGFBP3 commercial antibodies/Ecto-TMEM219 or left untreated (n=3) and in healthy controls (n=3). Monoclonal (clone 83.8F9) and polyclonal antibodies were used at a 1:10 ratio compared to Ecto-TMEM219. **p<0.01 Ecto-TMEM219 vs. IBD patient.

FIG. 5. IGFBP3-Ecto-TMEM219 binding in the presence of newly generated anti-IGFBP3 mAbs (n=11, YU139 antibodies) or Ecto-TMEM219 alone or monoclonal anti-IGFBP3 commercially available (LSBIO LS-C45037/clone 83.8F9, mAnti-IGFBP3) tested by using a competitive ELISA screening assay. In this assay, the microtiter plate was coated with rhlGFBP3, and labeled ecto-TMEM219 added. Each candidate monoclonal antibody was added and its ability to displace ecto-TMEM219 was assessed by measuring Absorbance after the plate was washed. Boxed are anti-IGFBP3 antibodies able to achieve the highest reduction in Ecto-TMEM219 signal (1-way ANOVA, p<0.00001).

FIG. 6. Effect of anti-IGFBP3 mAbs on mini-gut development upon IGFBP3 exposure. 4 out of 11 of newly generated mAbs significantly rescued mini-guts growth upon IGFBP3 exposure (boxed). *p<0.05, **p<0.01, ***p<0.001, mAbs/Ecto-TMEM219 vs. IGFBP3. Monoclonal anti-IGFBP3 antibodies were used with 1:1 molecular ratio to IGFBP3 (as Ecto-TMEM219). Commercial monoclonal anti-IGFBP3 antibody was purchased from LSBIO (LSBIO LS-C45037/107162) as described above.

FIG. 7. Effects of anti-IGFBP3 mAbs on mini-gut morphology upon IGFBP3 exposure. 11 newly generated anti-IGFBP3 mAbs, commercial monoclonal anti-IGFBP3 antibody (LSBIO, LS-C45037/107162) and Ecto-TMEM219 were tested on mini-guts treated with IGFBP3 and self-renewal properties were assessed by morphology evaluation. Development of large crypts organoids with at least one crypt domain was considered as main criteria. Unrelated antibody was anti-NLRP3 (IC7578A, R&D Systems). Original magnification X40.

FIG. 8A-8B. ISCs markers expression is re-established by newly generated anti-IGFBP3 mAbs in IGFBP3-treated minigut (n=3). Normalized mRNA expression of ISCs markers EphB2 (8A) and LGR5 (8B) analyzed by using RT-PCR in mini-guts cultured with IGFBP3 and selected anti-IGFBP3 mAbs/Ecto-TMEM219. *p<0.05, ** p<0.01 mAbs vs. IGFBP3.

FIG. 9. Caspase 8 expression is down-regulated by newly generated anti-IGFBP3 mAbs in IGFBP3-treated mini-guts. Normalized mRNA expression of Caspase 8 analyzed by using RT-PCR in mini-guts cultured with IGFBP3 and selected anti-IGFBP3 mAbs/Ecto-TMEM219. *p<0.05, ** p<0.01 mAbs vs. IGFBP3 (n=3).

FIG. 10. Effects of anti-IGFBP3 mAbs in rescuing mini-guts growth in DE (n=2). Mini-guts were grown with serum obtained from patients with diabetic enteropathy (DE) as described in the Methods and selected newly generated anti-IGFBP3 mAbs/Ecto-TMEM219 were added. The detrimental effect of on mini-guts growth induced by the DE-conditioning medium was significantly abrogated by some of the anti-IGFBP3 mAbs tested. * p<0.05, ** p<0.01 mAbs vs. DE medium.

FIG. 11. Effects of anti-IGFBP3 mAbs in rescuing mini-guts growth in IBD (n=3). Mini-guts were obtained from patients with IBD and re-challenged with/without IGFBP3 and newly generated anti-IGFBP3 mAbs/Ecto-TMEM219. Mini-guts development was rescued by some of the anti-IGFBP3 mAbs tested. *p<0.05, ** p<0.01, ***p<0.001 mAbs vs. IBD patient.

FIG. 12A-12G. Epitope binning experiments: (12A) IGFBP3 was directly coated on the plate (black line) or captured by the coated commercial antibody (LSBIO LS-C45037/clone 83.8F9) (red line). (12B, 12C, 12D, 12E, 12F, 12G) A dose-dependent binding is observed for all tested newly generated anti-IGFBP3 antibodies even when IGFBP3 is captured by the commercial LSBIO, LS-C45037/clone 83.8F9 antibody.

FIG. 13A-13B. FIG. 13A. Newly generated anti-IGFBP3 mAbs were tested for their ability to displace the IGF-I-IGFBP3 interaction by using a competitive ELISA in vitro. FIG. 13B. Briefly, plate was coated with IGFBP3 (Life Technologies) 4 μg/ml. IGF-I (R&D, 291-G1-01M) was added in a dose dependent ratio with IGFBP3 (0:1, 0.5:1, and 2:1). The anti-IGFBP3 mAbs (Yumab) were added at a concentration of 1:1 as compared to IGFBP3. IGFBP3-IGF-I binding was revealed by using anti-IGF-I HRP conjugated antibody. A commercial monoclonal anti-IGFBP3 antibodies (Novus Biological) was evaluated in the competitive ELISA binding assay as control.

FIG. 14. Newly generated monoclonal antibodies were tested in the murine mini-gut assay. Briefly, mini-guts were generated from crypts obtained from control mice C57BL6/J (n=3) and cultured for 8 days upon IGFBP3 exposure and treated with either ecto-TMEM219 or anti-newly generated IGFBP3 mAbs at a ratio of 1:1 (mAbs/ecto-TMEM219: IGFBP3).

FIG. 15A-15D. Effect of newly generated anti-IGFBP3 mAbs on DSS-induced colitis in mice (15A) Experimental timelines of DSS-induced colitis mice study, (15B) Disease activity index (DAI) score, Blood in Stool (15C) and Colon Size (15D) were evaluated 7 days after the last administration of DSS during the wound healing/beginning of the chronic phase.

FIG. 16A-16B. Effect of newly generated anti-IGFBP3 mAbs on type-1 diabetes mice model (16A) Experimental timelines of NOD mice study, (16B) Anti-IGFBP3 mAbs effect on incidence of type-1 diabetes in female NOD mice detected at 21 weeks of age, diabetes prevention by anti-IGFBP3 mAbs was observed in 75% of mice.

### DETAILED DESCRIPTION OF THE INVENTION

The antibodies of the invention include antibodies that specifically bind IGFBP3. As discussed herein, these antibodies are collectively referred to as “anti-IGFBP3 antibodies”. Thus, by “anti-IGFBP3 antibodies” is intended antibodies specific for IGFBP3. All of these antibodies are encompassed by the discussion herein. The respective antibodies can be used alone or in combination in the methods of the invention.

By “antibodies that specifically bind” is intended that the antibodies will not substantially cross react with another polypeptide. By “not substantially cross react” is intended that the antibody or fragment has a binding affinity for a non-homologous protein which is less than 10%, more preferably less than 5%, and even more preferably less than 1%, of the binding affinity for IGFBP3.

As used herein, the term “antigen binding molecule” refers in its broadest sense to a molecule that specifically binds an antigenic determinant. Examples of antigen binding molecules are antibodies, antibody fragments and scaffold antigen binding proteins. The term “antigen binding moiety” refers to a polypeptide molecule that specifically binds to an antigenic determinant. In one aspect, the antigen binding moiety is able to activate signaling through its target cell antigen. In a particular aspect, the antigen binding moiety is able to direct the entity to which it is attached to a target site. Antigen binding moieties include antibodies and fragments thereof capable of specific binding to a target cell antigen. In addition, antigen binding moieties capable of specific binding to a target cell antigen include scaffold antigen binding proteins as defined herein below, e.g. binding domains which are based on designed repeat proteins or designed repeat domains such as designed ankyrin repeat proteins (DARPins) (see e.g. WO 2002/020565) or Lipocalins (Anticalin).

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity. Additional terms are defined below and throughout the application.

The term “antibody” is intended to refer to immunoglobulin molecules comprising at least one heavy (H) chain or at least one light (L) chain. Said chains may be inter-connected by disulfide bonds. The term “antibody” also comprises multimers of said chains (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of at least one CDR, preferably three CDRs and at least one FR, preferably four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences or may be naturally or artificially modified. An amino acid consensus sequence may be defined based on a side-by-side analysis of two or more CDRs.

The term “antibody,” as used herein, also includes ADC (antibody drug conjugate), payload fusion antibodies, bispecific, Fab, scFv, diabodies, triabodies, minibodies and single domain antibodies, camelid IgG, IgNAR, as described in (25) incorporated by reference.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Designed Ankyrin Repeat Proteins (DARPins) are derived from Ankyrin which is a family of proteins that mediate attachment of integral membrane proteins to the cytoskeleton. A single ankyrin repeat is a 33-residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028.

A single-domain antibody is an antibody fragment consisting of a single monomeric variable antibody domain. The first single domain was derived from the variable domain of the antibody heavy chain from camelids (nanobodies or VHH fragments). Furthermore, the term single-domain antibody includes an autonomous human heavy chain variable domain (aVH) or VNAR fragments derived from sharks.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, and bivalent nanobodies), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric VH or VL domain (e.g., by disulfide bond(s)).

In certain embodiments, antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in antigen binding molecules may be made in order to create variants with certain improved properties. In one aspect, variants of antigen binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of antigen binding molecules of the invention include those with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the antigen binding molecule of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the antigen binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly (n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed. In another aspect, immunoconjugates of the antigen binding molecules provided herein may be obtained. An “immunoconjugate” is an antibody conjugated to one or more heterologous molecule(s), including but not limited to a cytotoxic agent.

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may in various embodiments be adapted for use in the context of an antigen-binding fragment of an anti-IL-6R antibody using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences.

The human antibodies featured in the invention may in various embodiments nonetheless include amino acid residues not encoded by human germline immunoglobulin sequences (e.g., mutations introduced by random or site-specific mutagenesis in vitro or by somatic mutation in vivo), for example in the CDRs and in in some embodiments CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295, incorporated herein by reference in its entirety,) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In another embodiment, the dimers are not linked via inter-chain disulfide bonds and a molecule of about 75-80 kDa is formed composed of a covalently coupled light and heavy chain (half-antibody). These embodiments/forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses in various embodiments antibodies having one or more mutations in the hinge, CH2 or CH3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” In various embodiments, the isolated antibody also includes an antibody in situ within a recombinant cell. In other embodiments, isolated antibodies are antibodies that have been subjected to at least one purification or isolation step. In various embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IGFBP3, as used herein, includes antibodies that bind IGFBP3 or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ) or kinetic exclusion assays.

The term “KD”, as used herein, is intended to refer to the equilibrium dissociation constant of an antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-IGFBP3 antibodies useful for the methods featured herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes in various embodiments methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes methods involving the use of anti-IGFBP3 antibodies comprising variants of any of the HCVR, LCVR, and/or CDR amino acid sequences disclosed herein having one or more conservative substitutions. For example, the present invention includes the use of anti-IL-6R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “bioequivalent” as used herein, refers to a molecule having similar bioavailability (rate and extent of availability) after administration at the same molar dose and under similar conditions (e.g., same route of administration), such that the effect, with respect to both efficacy and safety, can be expected to be essentially same as the comparator molecule. Two pharmaceutical compositions comprising an anti-IGFBP3 antibody are bioequivalent if they are pharmaceutically equivalent, meaning they contain the same amount of active ingredient (e.g., IGFBP3 antibody), in the same dosage form, for the same route of administration and meeting the same or comparable standards. Bioequivalence can be determined, for example, by an in vivo study comparing a pharmacokinetic parameter for the two compositions. Parameters commonly used in bioequivalence studies include peak plasma concentration (Cmax) and area under the plasma drug concentration time curve (AUC).

The invention in certain embodiments relates to methods comprising administering to the subject an antibody which comprises the heavy chain variable region comprising a sequence chosen from the group of: SEQ ID NO:55 to SEQ ID NO:65 and the light chain variable region comprising a sequence chosen from the group of: SEQ ID NO:66 to SEQ ID NO:76. The disclosure provides pharmaceutical compositions comprising such antibody, and methods of using these compositions.

The antibody is administered to the subject in various embodiments in a formulation comprising suitable carriers, excipients, and other agents to provide improved transfer, delivery, tolerance, and the like, and suitable for an intravenous or subcutaneous injection.

The injectable preparations may be prepared by methods publicly known. For example, injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 20 or 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injectable preparation thus prepared can be filled in an appropriate ampoule.

The antibody according to the invention can be administered to the subject using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer an antibody (or pharmaceutical formulation comprising the antibody). Examples of such devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen and/or autoinjector delivery devices having applications in subcutaneous delivery of a pharmaceutical composition of the present invention include, but are not limited to, the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), the HUMIRA™ Pen (Abbott Labs, Abbott Park, IL), the DAI® Auto Injector (SHL Group) and any auto-injector featuring the PUSHCLICK™ technology (SHL Group), to name only a few.

In one embodiment, the antibody is administered with a prefilled syringe. In another embodiment, the antibody is administered with a prefilled syringe containing a safety system. For example, the safety system prevents an accidental needlestick injury. In various embodiments, the antibody is administered with a prefilled syringe containing an ÈRIS™ safety system (West Pharmaceutical Services Inc.). See also U.S. Pat. Nos. 5,215,534 and 9,248,242, incorporated herein by reference in their entireties. In another embodiment, the antibody is administered with an auto-injector. In various embodiments, the antibody is administered with an auto-injector featuring the PUSHCLICK™ technology (SHL Group). In various embodiments, the auto-injector is a device comprising a syringe that allows for administration of a dose of the composition and/or antibody to a subject. See also U.S. Pat. Nos. 9,427,531 and 9,566,395, incorporated herein by reference in their entireties.

According to the invention, “subject” means a human subject or human patient.

### EXAMPLES

### Methods

### Patients and Study Design

Healthy subjects (CTRL) were enrolled (n=10) among patients undergoing to colonoscopy or intestinal surgery for diverticulosis, colon cancer, irritable bowel syndrome. Age: 41.3±2.2 (mean±SEM); Sex (M/F): 7/3.

IBD individuals had a 5-year history of Crohn's disease and were enrolled at the moment of surgery procedure for disease complications (strictures, fistulas) or during an endoscopy routine examination before undergoing surgery. Age: 47.1+3.1 (mean±SEM); Sex (M/F): 4/6.

DE serum was obtained from n=10 individuals with long-standing type 1 diabetes (history of diabetes >20 years), intestinal symptoms and motility dysfunction, with age ranging from 41 to 43 years old, and pooled for in vitro experiments. All subjects provided informed consent before study enrollment.

### Recombinant Proteins and Interventional Studies

Recombinant human IGFBP3 was obtained from Life Technologies (IGFBP3, Life Technologies, 10430H07H5). ecto-TMEM219 was obtained through Genescript's customized protein service. The protein, produced in E. coli, has the following amino acid sequence,

(SEQ ID NO: 104)
THRTGLRSPDIPQDWVSFLRSFGQLTLCPRNGTVTGKWRGSHVVGLLTT
LNFGDGPDRNKTRTFQATVLGSQMGLKGSSAGQLVLITARVTTERTAGT
CLYFSAVPGILPSSQPPISCSEEGAGNATLSPRMGEECVSVWSHEGLVL
TKLLTSEELALCGSR.

IGFBP3 at 50 ng/ml and ecto-TMEM219 at 130 ng/ml were added to culture medium at day +1 from mini-guts culture (see below).

Monoclonal and polyclonal anti-IGFBP3 (LSBio LS-C45037/107162 mouse IgG1 clone 83.8F9; LSBIO LS-C149975/72664, goat IgG) were added to culture medium at day +1 from mini-guts culture at 1, 10, 50 μg/ml and at 10 μg/ml final concentration respectively. Newly generated anti-IGFBP3 monoclonal antibodies were added at 1:1 molecular ratio as compared to IGFBP3 at 10 μg/ml final concentration.

### Crypts Isolation and Mini-Guts Development

Crypts were extracted from mucosa and sub-mucosa of intestinal samples of healthy subjects (healthy controls) or obtained from patients with established Crohn's disease undergoing surgery for disease complications (strictures, fistulae). Mucosa was incubated with a mixture of antibiotics Normocin, [Invivogen, San Diego, California 92121, USA ant-nr], Gentamycin [Invitrogen, Carlsbad, CA, USA ant-gn] and Fungizone [Invitrogen 15290018]) for 15 minutes at room temperature, and then tissue was minced into small pieces and incubated with 10 mM Dithiothreitol (DTT) (Sigma) in PBS 2-3 times for several minutes. Samples were then transferred to 8 mM EDTA in PBS and incubated for 30 minutes at 37° C. After this step, vigorous shaking of the sample yielded supernatants enriched in colonic crypts. Fetal bovine serum (FBS, Sigma 12103C-500ML) was added to a final concentration of 5%, and single cells were removed by centrifugation 40×g for 2 minutes. Crypts were mixed with 50 μl of Matrigel (BD Biosciences 354234) and plated on pre-warmed culture dishes. After solidification, crypts were overlaid with complete crypt culture medium: Wnt3a-conditioned medium and Advanced DMEM/F12 (Life Technologies 1263010) 50:50, supplemented with Glutamax, 10 mM (Life Technologies 35050038) HEPES (Life Technologies 15630080), N-2 [1×] (Life Technologies 17502048), B-27 without retinoic acid [1×] (Life Technologies 12587010), 10 mM Nicotinamide (Sigma N0636), 1 mM N-Acetyl-L-cysteine (Sigma A965), 50 ng/ml human EGF (Life Technologies PHG0311), 1 μg/ml RSPO1 (Sino Biological 11083-H08H), 100 ng/ml human Noggin (Peprotech 12010C), 1 μg/ml Gastrin (Sigma-Aldrich SCP0152), 500 nM LY2157299 (Axon MedChem 1491), 10 uM SB202190 (Sigma S7067) and 0.01 μM PGE2 (Sigma P6532). Isolated crypts have been cultured for 8 days with/without recombinant proteins/Antibodies as described in the Recombinant proteins and interventional studies section. After 8 days, crypts were collected, and the morphology, mini-gut growth, expression of intestinal signature markers (EphB2, LGR5, h-TERT), and Caspase 8 (Life Technologies) were examined using RT-PCR. Percentage of developed mini-guts with at least one crypt domain was assessed as already described (4,18).

### In Vitro Mini-Gut Generation Study

Crypts were isolated from healthy subject biopsy samples and cultured as previously described to generate mini-guts. To mimic the pathologic conditions, the crypt culturing medium was modified by adding human serum of individuals with diabetic enteropathy (DE), namely “DE” medium, in place of regular FBS, L-Wnt3 cells were grown in 10% “DE” serum to generate conditioned medium that was further added 50:50 to Advanced DMEM/F12 medium in order to obtain the crypts culture medium as described above (see Crypts isolation and mini-guts development). After 8 days, crypts were collected, and the morphology, mini-gut growth, expression of intestinal signature markers (EphB2, LGR5, h-TERT), and Caspase 8 (Life Technologies) were examined using RT-PCR. This has been established as model of intestinal stem cells-damage (4).

qRT-PCR Analysis

RNA from purified intestinal crypts was extracted using Trizol Reagent (Invitrogen), and qRT-PCR analysis was performed using TaqMan assays (Life Technologies, Grand Island, NY) according to the manufacturer's instructions. The normalized expression values were determined using the ΔΔCt or the ΔCt method. Quantitative reverse transcriptase polymerase chain reaction (qRT-PCR) data were normalized for the expression of ACTB, and ΔCt values were calculated. Analysis was performed in technical and biological triplicates.

The list of genes which expression have been quantified by qRT-PCR is reported below.

TABLE 1

| Gene | | Refseq | Band Size | Reference |
|---|---|---|---|---|
| Symbol | UniGene # | Accession # | (bp) | Position |
| LGR5 | Hs.658889 | NM_003667 | 91 | 1665 |
| EPHB2 | Hs.523329 | NM_004442 | 68 | 2908 |
| TERT | Hs.492203 | NM_198253 | 106 | 1072 |
| ACTB | Hs.520640 | NM_001101 | 174 | 730 |
| CasDase 8 | Hs.599762 | NM_001080124.1 | 124 | 648 |

### Competitive ELISA Binding Assay

The following reagents were used to screen the newly generated anti-IGFBP3 antibodies: Recombinant Human IGFBP3 (0.223 mg/ml R&D System 8874-B3-025), Ecto-TMEM219 (0.5 mg/ml GenScript), newly generated anti-IGFBP3 mAbs (Yumab, under contract), goat polyclonal anti-IGFBP3 (LS-C149975 LSBio), mouse monoclonal anti-IGFBP3 (LS-C45037 LSBio), mouse 6×His tag HRP (GTX30506 Genetex), bovine serum albumin (BSA A9418 Sigma), Tween 20 (TW P2287 Sigma), ELISA colorimetric TMB reagent (HRP substrate, Item H Sigma, RABTMB3), ELISA STOP solution (Item I, Sigma, RABSTOP3), blocking reagent solution (3% BSA in PBS) and a diluent solution (0,5% BSA, 0.05% Tw in PBS).

Microplate was coated with 50 μl/well of 4 μg/ml rhlGFBP3 dissolved in PBS or PBS alone (no coating). Plate was incubated 90 minutes at 37° C. and washed with PBS (300 μl/well) and incubated with the blocking reagent (200 μl/well) 2 hours at room temperature. Samples were then diluted in the diluent solution (50 μl/well) and added to the plate as follows: diluent solution (none), ecto-TMEM 10 μg/ml, ecto-TMEM 10 μg/ml+anti-IGFBP3 mAbs 10 μg/ml, anti-IGFBP3 mAbs 10 μg/ml alone. After washing steps, plate was then incubated at room temperature for 1 hour with anti 6×His tag HRP diluted 1:2000 in Diluent solution (50 μl/well). ELISA plate was then read after adding visualization solution at ELISAreader and adsorbance was measured.

### Epitope Binning

Epitope binning is a competitive immunoassay used to characterize and then sort a library of monoclonal antibodies against a target protein (Abdiche, Y. N.; D. S. Malashock; A. Pinkerton; J. Pons (March 2009). “Exploring blocking assays using Octet, ProteOn, and Biacore biosensors”. Analytical Biochemistry. 386 (2): 172-180. doi:10.1016/j.ab.2008.11.038. PMID 19111520). Antibodies against a similar target are tested against all other antibodies in the library in a pairwise fashion to see if antibodies block one another's binding to the epitope of an antigen. After each antibody has a profile created against all of the other antibodies in the library, a competitive blocking profile is created for each antibody relative to the others in the library. Closely related binning profiles indicate that the antibodies have the same or a closely related epitope and are “binned” together. Epitope binning is referenced in the literature under different names such as epitope mapping and epitope characterization (Brooks, B. D. (2014). “The Importance of Epitope Binning in Drug Discovery”. Current Drug Discovery Technology. 11.).

CVC microplates were coated with: i) 100 μl/well of 2 μg/ml rhIGFBP3 dissolved in PBS or PBS alone (no coating) and incubated 60 minutes at RT; ii) 100 μl/well of 2 μg/ml anti-IGFBP3 (LS-C45037 LSBio) and incubated o.n at 4° C. Plates were washed with H₂O-Tween20 (0.05%) and incubated with the blocking reagent (2% BSA-PBS-T (0.05%)). For condition ii) 100 μl/well of 2 μg/ml rhIGFBP3 was incubated o.n at 4° C. After washing the newly generated antibodies (in 2% BSA-PBS-T (0.05%)) were titrated onto the plate and incubated for 1 hour at RT. After washing steps, detection of bound antibodies was performed using a goat anti-human Fc-HRP antibody (Sigma A0170) in 2% BPS-PBS-T (0.05%) followed by washing and read out with TMB (100 μl/well). ELISA plates were then read measuring the absorbance at 450 nm.

### Beta-Cells

Betalox-5 cells, a human beta cell line (36) were grown in culture flasks containing DMEM (glucose 1 g/L), BSA fraction V (0.02% wt/vol), Non-essential amino acids (1×) penicillin (100 units/mL), and streptomycin (100 μg/mL). The cells were cultured at 37° C. in a humidified incubator in 5% CO2. The cells were passaged once every second week. Beta cells were cultured with or without IGFBP3, with or without ecto-TMEM219, with or without newly generated monoclonal antibodies (see Recombinant proteins and interventional studies) and cells were collected for immunofluorescence studies, RNA extraction, apoptosis detection, and protein analysis. Supernatants were collected for assessment of insulin. Insulin levels were assayed with a microparticle enzyme immunoassay (Mercodia Iso-Insulin ELISA, 10-1113-01).

### Statistical Analysis

Data are presented as mean and standard deviation (SD) and were tested for normal distribution with the Kolmogorov-Smirnov test and for homogeneity of variances with Levene's test. The statistical significance of differences was tested with two-tailed t-test and the chi-square (χ2) tests. Significance between the two groups was determined by two-tailed unpaired Student's t test. For multiple comparisons, the 1-way ANOVA test with Bonferroni correction was employed. Statistical analysis was conducted using GraphPad Prism version 5.0 (GraphPad Software, La Jolla, CA). All statistical tests were performed at the 5% significance level.

### Example 1

### Monoclonal Antibodies Development

Monoclonal anti-IGFBP3 antibodies were discovered from naïve human phage-display libraries using a recombinant full length human IGFBP3 (R&D cat n° 8874-B3) as antigen for the screening. Briefly, human IGFBP3 (R&D cat n° 8874-B3) was biotinylated and immobilized via streptavidin onto 96-well ELISA plates at 4° C. After washing and blocking of the wells with BSA, the antibody-phage libraries were added. Tested libraries were first cleared from antibody-phage that bind to Streptavidin (John deKruif and Ton Logtenberg, Phage Display of Antibodies, Encyclopedia of Immunology (Second Edition) 1998, Pages 1931-1934). Those phages that carried an antigen-specific antibody were captured on the plate surface. After removal by washing with PBS-T of unbound/weakly bound phage with, antigen-specific phage were eluted and amplified by phage infection in E. coli. This amplified library subset was again selected for target binding under more stringent conditions. In total, three selection rounds were performed to enrich antigen specific antibody-phage.

At the end of the discovery process, each selection output was screened for antigen-specific antibodies. For this purpose, 384 clones were chosen from each selection output and used for production of monoclonal scFv antibodies. These were then tested for specific antigen binding by ELISA. 24 target specific hits were identified which were sequenced. All antibodies showing unique CDRs sequences were cloned into a mammalian scFv-Fc expression vector. As a result, the scFv was genetically fused to a Fc Fragment of human IgG4.

This resulted in 11 scFv-Fc antibodies which were produced by transient transfection of HEK293 cells. Then, the antibodies were purified by affinity chromatography (Protein A) and re-buffered in PBS (no additives). The protein concentration was determined by UV/VIS spectrometry and purity was checked by Coomassie staining.

The sequences of the 11 novel anti-IGFBP3 antibodies are reported in the Tables below.

TABLE 2
VH CDR Sequences of exemplified antibodies

| Anti- | VH | | |
|---|---|---|---|
| body | CDR1 | VH CDR2 | VH CDR3 |
| YU139- | SYAIS | GIIPIFGTANYAQKFQG | DYYDSSGYYFDAFDI |
| A02 | (SEQ ID | (SEQ ID NO: 8) | (SEQ ID NO: 17) |
| | NO: 1) | | |
| YU139- | SYGIH | IVSYDGRHKYYADSVKG | DSGNSGLEGIHDY |
| A03 | (SEQ ID | (SEQ ID NO: 9) | (SEQ ID NO: 18) |
| | NO: 2) | | |
| YU139- | SSNWWS | EVYHSGSTNYNPSLKS | NIPLSSSWPNYYYYY |
| B01 | (SEQ ID | (SEQ ID NO: 10) | GMDV |
| | NO: 3) | | (SEQ ID NO: 19) |
| YU139- | SYGIS | WINTYNGNTNYAQKLQG | DIGYSGSYYSPYYYY |
| C01 | (SEQ ID | (SEQ ID NO: 11) | GMDV |
| | NO: 4) | | (SEQ ID NO: 20) |
| YU139- | SYGIS | WISAYNGNTNYAQKLQG | VPSEYYDFWSGYYTE |
| C02 | (SEQ ID | (SEQ ID NO: 12) | ENAFDI |
| | NO: 4) | | (SEQ ID NO: 21) |
| YU139- | SYGMH | VISYDGSNKYYADSVKG | DNGDYGYYYYYYMDV |
| D02 | (SEQ ID | (SEQ ID NO: 13) | (SEQ ID NO: 22) |
| | NO: 5) | | |
| YU139- | DYGIS | WISGDNVKTTYAKKFQG | GGIVFDY |
| D03 | (SEQ ID | (SEQ ID NO: 14) | (SEQ ID NO: 23) |
| | NO: 6) | | |
| YU139- | SYGIS | WISAYNGNTNYAQKLQG | GGIVFDY |
| F02 | (SEQ ID | (SEQ ID NO: 12) | (SEQ ID NO: 23) |
| | NO: 4) | | |
| YU139- | SYGIS | WISAYNGNTYYAQKLQG | LSYYNYAMDV |
| G01 | (SEQ ID | (SEQ ID NO: 15) | (SEQ ID NO: 24) |
| | NO: 4) | | |
| YU139- | TYVTS | GIIPMFDTTEYAQKLEG | AGGLSHFDY |
| G03 | (SEQ ID | (SEQ ID NO: 16) | (SEQ ID NO: 25) |
| | NO: 7) | | |
| YU139- | SYGIS | WISAYNGNTNYAQKLQG | VKWELGWAFDI |
| H03 | (SEQ ID | (SEQ ID NO: 12) | (SEQ ID NO: 26) |
| | NO: 4) | | |

TABLE 3
VL CDR sequences of exemplified antibodies

| Anti- | | VL | |
|---|---|---|---|
| body | VL CDR1 | CDR2 | VL CDR3 |
| YU139- | SGSSSNIGTNTVS | NNNERPS | AAWDDNLTGLV |
| A02 | (SEQ ID NO: 27) | (SEQ ID | (SEQ ID |
| | | NO: 37) | NO: 45) |
| YU139- | RASQGISSWLA | AASSLQS | QQANSFPLT |
| A03 | (SEQ ID NO: 28) | (SEQ ID | (SEQ ID |
| | | NO: 38) | NO: 46) |
| YU139- | QGDSLRNYYAS | GKNNRPS | NSRDSSAKHWV |
| B01 | (SEQ ID NO: 29) | (SEQ ID | (SEQ ID |
| | | NO: 39) | NO: 47) |
| YU139- | QGDGLRNYFAS | GKNNRPS | NSRDGSGKHLV |
| C01 | (SEQ ID NO: 30) | (SEQ ID | (SEQ ID |
| | | NO: 39) | NO: 48) |
| YU139- | RASQSISSYLN | AASSLQS | QQSYSTWT |
| C02 | (SEQ ID NO: 31) | (SEQ ID | (SEQ ID |
| | | NO: 38) | NO: 49) |
| YU139- | RASQSISRWLA | DASSLES | QQSYSTPLT |
| D02 | (SEQ ID NO: 32) | (SEQ ID | (SEQ ID |
| | | NO: 40) | NO: 50) |
| YU139- | RASQSVSGNYLA | GASSRAT | QQYGSSPGT |
| D03 | (SEQ ID NO: 33) | (SEQ ID | (SEQ ID |
| | | NO: 41) | NO: 51) |
| YU139- | RASQSISSYLN | AASSLQS | QQSYSTPLT |
| F02 | (SEQ ID NO: 31) | (SEQ ID | (SEQ ID |
| | | NO: 38) | NO: 50) |
| YU139- | TGTSSDVGGYNHVS | EVSNRPS | SSYTSSNTWV |
| G01 | (SEQ ID NO: 34) | (SEQ ID | (SEQ ID |
| | | NO: 42) | NO: 52) |
| YU139- | RSSQSLLHSNGNNYLN | LSSRRAS | MQGLQTPWT |
| G03 | (SEQ ID NO: 35) | (SEQ ID | (SEQ ID |
| | | NO: 43) | NO: 53) |
| YU139- | TGSSSNIGAGYDVH | GNSNRPS | QSYDSSLSTV |
| H03 | (SEQ ID NO: 36) | (SEQ ID | (SEQ ID |
| | | NO: 44) | NO: 54) |

TABLE 4
VH amino acid sequences of exemplified antibodies

| Anti- | |
|---|---|
| body | AA of VH |
| YU139- | QVQLVQSGAEVKKPGSSVKVSCKASGGTFSSYAISWVRQ |
| A02 | APGQGLEWMGGIIPIFGTANYAQKFQGRVTITADESTST |
| | AYMELSSLRSEDTAVYYCARDYYDSSGYYFDAFDIWGQG |
| | TMVTVSS(SEQ ID NO: 55) |
| YU139- | QVQLVQSGGGVVQPGRSLRLSCAASGFTFSSYGIHWVRQ |
| A03 | APGKGLEWVAIVSYDGRHKYYADSVKGRFTISRDDSKNT |
| | IYLQMDSLRAEDTAVYYCAKDSGNSGLEGIHDYWGQGTL |
| | VTVSS (SEQ ID NO: 56) |
| YU139- | QVQLQESGPGLVKPSGTLSLTCAVSGGSISSSNWWSWVR |
| B01 | QPPGKGLEWIGEVYHSGSTNYNPSLKSRVTISVDKSKNQ |
| | FSLKLSSVTAADTAVYYCARNIPLSSSWPNYYYYYGMDV |
| | WGQGTTVTVSS (SEQ ID NO: 57) |
| YU139- | QVQLVQSGAEVKKPGASVKVSCKASGYTFTSYGISWVRQ |
| C01 | APGQGLEWMGWINTYNGNTNYAQKLQGRVTMTTDTSTTT |
| | AYMELRSLRSDDTAVYYCARDIGYSGSYYSPYYYYGMDV |
| | WGQGTTVTVSS (SEQ ID NO: 58) |
| YU139- | EVQLVQSGAEVKKPGASVKVSCKASGYTFTSYGISWVRQ |
| C02 | APGQGLEWMGWISAYNGNTNYAQKLQGRVTMTTDTSTST |
| | AYMELRSLRSDDTAVYYCARVPSEYYDFWSGYYTEENAF |
| | DIWGQGTMVTVSS (SEQ ID NO: 59) |
| YU139- | EVQLVESGGGVVQPGRSLRLSCAASGFTFSSYGMHWVRQ |
| D02 | APGKGLEWVAVISYDGSNKYYADSVKGRFTISRDNSKNT |
| | LYLQMNSLRAEDTAVYYCAKDNGDYGYYYYYYMDVWGKG |
| | TTVTVSS (SEQ ID NO: 60) |
| YU139- | QVQLVQSGAEVKKPGASVKVSCKASGYTFSDYGISWVRQ |
| D03 | APGQGLEWMGWISGDNVKTTYAKKFQGRVTLTTDTSTST |
| | AYMELRSLTSDDTAAYYCARGGIVFDYWGQGTLVTVSS |
| | (SEQ ID NO: 61) |
| YU139- | QVQLVQSGAEVKKPGASVKVSCKASGYTFTSYGISWVRQ |
| F02 | ATGQGLEWMGWISAYNGNTNYAQKLQGRVTMTTDTSTST |
| | AYMELRSLRSDDTAVYYCARGGIVFDYWGQGTLVTVSS |
| | (SEQ ID NO: 62) |
| YU139- | QMQLVQSGAEVKMPGASVKVSCKASGYTFTSYGISWVRQ |
| G01 | APGQGLEWMGWISAYNGNTYYAQKLQGRVTMTADTSTST |
| | AYMDLRSLRSDDTAVYYCARLSYYNYAMDVWGQGTTVTV |
| | SS (SEQ ID NO: 63) |
| YU139- | QVQLQQSGAEVKKPGSSVKVSCKASGGIFSTYVTSWVRQ |
| G03 | APGQGLEWMGGIIPMFDTTEYAQKLEGRVTITVDESTNT |
| | AYMELSSLRFEDTAVYYCARAGGLSHFDYWGQGTLVTVS |
| | S (SEQ ID NO: 64) |
| YU139- | QVQLVQSGAEVKKPGASVKVSCKASGYTFTSYGISWVRQ |
| H03 | APGQGLEWMGWISAYNGNTNYAQKLQGRVTMTTDTSTST |
| | AYMELRSLRSDDTAVYYCARVKWELGWAFDIWGQGTMVT |
| | VSS (SEQ ID NO: 65) |

TABLE 5
VL amino acid sequences of exemplified antibodies

| Anti- | |
|---|---|
| body | AA of VL |
| YU139- | QAGLTQPPSASGTPGQRVIISCSGSSSNIGTNTVSWYQH |
| A02 | LPGTAPKLLIYNNNERPSGVPRRFSGSRSGASASLAISG |
| | LQSDDEAHYYCAAWDDNLTGLVFGGGTKLTVL |
| | (SEQ ID NO: 66) |
| YU139- | DVVMTQSPSSVSASVGDRVTITCRASQGISSWLAWYQQK |
| A03 | PGKAPKLLIYAASSLQSGVPSRFSGSGSGTDFTLTISSL |
| | QPEDFGTYYCQQANSFPLTFGGGTKVEIK |
| | (SEQ ID NO: 67) |
| YU139- | SSELTQDPAVSVALGQTVRITCQGDSLRNYYASWYQQKP |
| B01 | GQAPVLVIYGKNNRPSGIPDRFSGSSSGNTASLTINGAQ |
| | AEDEADYYCNSRDSSAKHWVFGGGTKLTV |
| | (SEQ ID NO: 68) |
| YU139- | SSELTQDPAVSVALGQTVRITCQGDGLRNYFASWYQQKP |
| C01 | GQAPVLVIYGKNNRPSGIPDRFSGSSSGNTASLRIAGAQ |
| | AEDEADYYCNSRDGSGKHLVFGGGTKLTV |
| | (SEQ ID NO: 69) |
| YU139- | DIVMTQSPSSLSASVGDRVTITCRASQSISSYLNWYQQK |
| C02 | PGKAPKLLIYAASSLQSGVPSRFSGSGSGTDFTLTISSL |
| | QPEDFATYYCQQSYSTWTFGQGTKVEIK |
| | (SEQ ID NO: 70) |
| YU139- | DIQMTQSPSTLSASVGDRVTITCRASQSISRWLAWYQQK |
| D02 | PGKAPKLLIYDASSLESGVPSRFSGSGSGTEFTLTISSL |
| | QPDDFATYYCQQSYSTPLTFGPGTKVDIK |
| | (SEQ ID NO: 71) |
| YU139- | ETTLTQSPGTLSLSPGERATLSCRASQSVSGNYLAWYQQ |
| D03 | KPGQPPRLLIFGASSRATGIPDRFSGSGSGTDFTLTISR |
| | LEPEDFGVYYCQQYGSSPGTFGQGTKVEIK |
| | (SEQ ID NO: 72) |
| YU139- | DVVMTQSPSSLSASVGDRVTITCRASQSISSYLNWYQQK |
| F02 | PGKAPKLLIYAASSLQSGVPSRFSGSGSGTDFTLTISSL |
| | QPEDFATYYCQQSYSTPLTFGGGTKVEIK |
| | (SEQ ID NO: 73) |
| YU139- | QSALTQPPSASGSPGQSVTISCTGTSSDVGGYNHVSWYQ |
| G01 | QHPGKAPKLMIYEVSNRPSGVPNRFSGSKSGNTASLTIS |
| | GLQAEDEADYYCSSYTSSNTWVFGGGTKLTVL |
| | (SEQ ID NO: 74) |
| YU139- | EIVLTQSPLSLPVTPGEPASISCRSSQSLLHSNGNNYLN |
| G03 | WYLQKPGQSPQLLIYLSSRRASGVPDRFSGSGSGTDFTL |
| | KISRVEAEDVGVYYCMQGLQTPWTFGQGTKVETK |
| | (SEQ ID NO: 75) |
| YU139- | QAVLTQPPSVSGAPGQRVTISCTGSSSNIGAGYDVHWYQ |
| H03 | QLPGTAPKLLIYGNSNRPSGVPDRFSGSKSGTSAPLAIT |
| | GLQAEDEADYYCQSYDSSLSTVFGGGTKLTVL |
| | (SEQ ID NO: 76) |

TABLE 6
VH nucleotide sequences of exemplified antibodies

| Anti- | |
|---|---|
| body | DNA of VH |
| YU139- | CAGGTTCAGCTGGTGCAGTCTGGGGCTGAGGTGAAGAAGC |
| A02 | CTGGGTCCTCGGTGAAGGTCTCCTGCAAGGCTTCTGGAGG |
| | CACCTTCAGCAGCTATGCTATCAGCTGGGTGCGACAGGCC |
| | CCTGGACAAGGGCTTGAGTGGATGGGAGGGATCATCCCTA |
| | TCTTTGGTACAGCAAACTACGCACAGAAGTTCCAGGGCAG |
| | AGTCACGATTACCGCGGACGAATCCACGAGCACAGCCTAC |
| | ATGGAGCTGAGCAGCCTGAGATCTGAGGACACGGCCGTGT |
| | ATTACTGTGCGAGAGATTACTATGATAGTAGTGGTTATTA |
| | CTTTGATGCTTTTGATATCTGGGGCCAAGGGACAATGGTC |
| | ACCGTCTCTTCA (SEQ ID NO: 77) |
| YU139- | CAGGTCCAGCTGGTACAATCTGGGGGAGGCGTGGTCCAGC |
| A03 | CTGGGAGGTCCCTGAGACTCTCCTGTGCAGCCTCTGGATT |
| | CACCTTCAGTAGCTATGGCATCCACTGGGTCCGCCAGGCT |
| | CCAGGCAAGGGGCTGGAGTGGGTGGCAATTGTATCATATG |
| | ATGGAAGACATAAATATTATGCAGACTCCGTGAAGGGCCG |
| | ATTCACCATCTCCAGAGACGATTCCAAGAACACGATCTAT |
| | CTGCAAATGGACAGCCTGAGAGCTGAGGACACGGCTGTAT |
| | ATTACTGTGCGAAAGATAGTGGGAACTCCGGTCTGGAAGG |
| | TATCCATGACTACTGGGGCCAGGGAACCCTGGTCACCGTC |
| | TCCTCA (SEQ ID NO: 78) |
| YU139- | CAGGTGCAGCTGCAGGAGTCGGGCCCAGGACTGGTGAAGC |
| B01 | CTTCGGGGACCCTGTCCCTCACCTGCGCTGTCTCTGGTGG |
| | CTCCATCAGCAGTAGTAACTGGTGGAGTTGGGTCCGCCAG |
| | CCCCCAGGGAAGGGGCTGGAGTGGATTGGGGAAGTCTATC |
| | ATAGTGGGAGCACCAACTACAACCCGTCCCTCAAGAGTCG |
| | AGTCACCATATCAGTAGACAAGTCCAAGAACCAGTTCTCC |
| | CTGAAGCTGAGCTCTGTGACCGCCGCGGACACGGCCGTGT |
| | ATTACTGTGCGAGAAACATCCCCCTTAGCAGCAGCTGGCC |
| | TAATTACTACTACTACTACGGTATGGACGTCTGGGGCCAA |
| | GGGACCACGGTCACCGTCTCCTCA (SEQ ID NO: 79) |
| YU139- | CAGGTCCAGCTGGTGCAATCTGGAGCTGAGGTGAAGAAGC |
| C01 | CTGGGGCCTCAGTGAAGGTCTCCTGCAAGGCTTCTGGCTA |
| | CACCTTTACCAGCTATGGTATCAGCTGGGTGCGACAGGCC |
| | CCTGGACAAGGGCTTGAGTGGATGGGATGGATCAACACTT |
| | ACAATGGTAACACAAACTATGCACAGAAACTCCAGGGCAG |
| | AGTCACCATGACCACAGACACATCCACGACCACAGCCTAC |
| | ATGGAGCTGAGGAGCCTGAGATCTGACGACACGGCCGTAT |
| | ATTACTGTGCGAGAGATATCGGGTATAGTGGGAGCTACTA |
| | TTCGCCCTACTACTACTACGGTATGGACGTCTGGGGCCAA |
| | GGGACCACGGTCACCGTCTCCTCA (SEQ ID NO: 80) |
| YU139- | GAGGTGCAGCTGGTGCAGTCTGGGGCTGAGGTGAAGAAGC |
| C02 | CTGGGGCTTCAGTGAAGGTCTCCTGCAAGGCTTCTGGTTA |
| | CACCTTTACCAGCTATGGTATCAGCTGGGTGCGACAGGCC |
| | CCTGGACAAGGGCTTGAGTGGATGGGATGGATCAGCGCTT |
| | ACAATGGTAACACAAACTATGCACAGAAGCTCCAGGGCAG |
| | AGTCACCATGACCACAGACACATCCACGAGCACAGCCTAC |
| | ATGGAGCTGAGGAGCCTGAGATCTGACGACACGGCTGTGT |
| | ATTACTGTGCGAGAGTTCCCTCAGAGTATTACGATTTTTG |
| | GAGTGGTTATTATACCGAAGAGAATGCTTTTGATATCTGG |
| | GGCCAAGGGACAATGGTCACCGTCTCTTCA |
| | (SEQ ID NO: 81) |
| YU139- | GAGGTGCAGCTGGTGGAGTCTGGGGGAGGCGTGGTCCAGC |
| D02 | CTGGGAGGTCCCTGAGACTCTCCTGTGCAGCCTCTGGATT |
| | CACCTTCAGTAGCTATGGCATGCACTGGGTCCGCCAGGCT |
| | CCAGGCAAGGGGCTGGAGTGGGTGGCAGTTATATCATATG |
| | ATGGAAGTAATAAATACTATGCAGACTCCGTGAAGGGCCG |
| | ATTCACCATCTCCAGAGACAATTCCAAGAACACGCTGTAT |
| | CTGCAAATGAACAGCCTGAGAGCTGAGGACACGGCTGTGT |
| | ATTACTGTGCGAAAGATAACGGTGACTACGGATACTACTA |
| | CTACTACTACATGGACGTCTGGGGCAAAGGGACCACGGTC |
| | ACCGTCTCCTCA (SEQ ID NO: 82) |
| YU139- | CAGGTGCAGCTGGTGCAGTCTGGAGCTGAGGTGAAGAAGC |
| D03 | CTGGGGCCTCAGTGAAGGTCTCCTGCAAGGCTTCTGGTTA |
| | CACCTTTTCCGACTATGGTATCAGCTGGGTGCGACAGGCC |
| | CCTGGACAAGGGCTTGAGTGGATGGGATGGATCAGCGGTG |
| | ACAATGTTAAGACTACCTATGCAAAGAAGTTCCAGGGCAG |
| | AGTCACCCTGACCACAGACACATCCACGAGCACAGCCTAC |
| | ATGGAGCTGAGGAGCCTGACATCTGACGACACGGCCGCAT |
| | ATTACTGTGCGAGAGGGGGGATCGTATTTGACTACTGGGG |
| | CCAGGGAACCCTGGTCACCGTCTCCTCA |
| | (SEQ ID NO: 83) |
| YU139- | CAGGTTCAGCTGGTGCAGTCTGGGGCTGAGGTGAAGAAGC |
| F02 | CTGGGGCCTCAGTGAAGGTCTCCTGCAAGGCTTCTGGTTA |
| | CACCTTTACCAGCTATGGTATCAGCTGGGTGCGACAGGCC |
| | ACTGGACAAGGGCTTGAGTGGATGGGATGGATCAGCGCTT |
| | ACAATGGTAACACAAACTATGCACAGAAGCTCCAGGGCAG |
| | AGTCACCATGACCACAGACACATCCACGAGCACAGCCTAC |
| | ATGGAGCTGAGGAGCCTGAGATCTGACGACACGGCCGTGT |
| | ATTACTGTGCGAGAGGGGGGATCGTATTTGACTACTGGGG |
| | CCAGGGAACCCTGGTCACCGTCTCCTCA |
| | (SEQ ID NO: 84) |
| YU139- | CAAATGCAGCTGGTACAGTCTGGGGCTGAGGTGAAGATGC |
| G01 | CTGGGGCCTCAGTGAAGGTCTCCTGCAAGGCTTCTGGTTA |
| | CACCTTTACCAGCTATGGTATCAGCTGGGTGCGACAGGCC |
| | CCTGGACAAGGGCTTGAGTGGATGGGGTGGATCAGCGCTT |
| | ACAATGGTAACACATACTATGCACAGAAACTCCAGGGCAG |
| | AGTCACCATGACCGCAGACACATCCACGAGCACAGCCTAC |
| | ATGGACCTGAGGAGCCTGAGATCTGACGACACGGCCGTGT |
| | ATTACTGTGCGAGACTATCATATTACAACTACGCTATGGA |
| | CGTCTGGGGCCAAGGGACCACGGTCACCGTCTCCTCA |
| | (SEQ ID NO: 85) |
| YU139- | CAGGTACAGCTGCAGCAGTCAGGGGCTGAGGTGAAGAAGC |
| G03 | CTGGGTCCTCGGTGAAGGTGTCCTGCAAGGCTTCTGGAGG |
| | CATCTTCAGCACCTATGTGACCAGCTGGGTGCGACAGGCC |
| | CCTGGACAAGGGCTTGAGTGGATGGGAGGGATCATCCCTA |
| | TGTTTGACACAACAGAGTACGCACAGAAGCTCGAGGGCAG |
| | AGTCACGATCACCGTGGACGAATCCACGAACACAGCCTAC |
| | ATGGAGCTGAGCAGCCTGAGATTTGAGGACACGGCCGTAT |
| | ATTACTGTGCGAGAGCGGGAGGTTTGAGCCACTTTGACTA |
| | CTGGGGCCAGGGAACTCTGGTCACCGTCTCGTCA |
| | (SEQ ID NO: 86) |
| YU139- | CAGGTCCAGCTGGTACAGTCTGGAGCTGAGGTGAAGAAGC |
| H03 | CTGGGGCCTCAGTGAAGGTCTCCTGCAAGGCTTCTGGTTA |
| | CACCTTTACCAGCTATGGTATCAGCTGGGTGCGACAGGCC |
| | CCTGGACAAGGGCTTGAGTGGATGGGATGGATCAGCGCTT |
| | ACAATGGTAACACAAACTATGCACAGAAGCTCCAGGGCAG |
| | AGTCACCATGACCACAGACACATCCACAAGCACAGCCTAC |
| | ATGGAGCTGAGGAGCCTGAGATCTGACGACACGGCCGTGT |
| | ATTACTGTGCGAGAGTGAAGTGGGAGCTAGGGTGGGCTTT |
| | TGATATCTGGGGCCAAGGGACAATGGTCACCGTCTCTTCA |
| | (SEQ ID NO: 87) |

TABLE 7
VL nucleotide sequences

| Anti- | |
|---|---|
| body | DNA of VL |
| YU139- | CAGGCTGGGCTGACTCAGCCACCCTCAGCGTCTGGGACCC |
| A02 | CCGGGCAGAGGGTCATCATCTCTTGTTCTGGAAGCAGCTC |
| | CAACATCGGAACTAATACTGTAAGCTGGTATCAACACCTC |
| | CCAGGAACGGCCCCCAAGCTCCTCATCTATAACAATAATG |
| | AACGGCCCTCAGGGGTCCCTCGCCGATTCTCTGGCTCCAG |
| | GTCTGGCGCCTCAGCCTCCCTGGCCATCAGTGGGCTCCAG |
| | TCTGACGATGAGGCTCATTATTATTGTGCAGCCTGGGATG |
| | ACAACCTGACTGGCTTGGTGTTCGGCGGAGGGACCAAACT |
| | GACCGTCCTA (SEQ ID NO: 88) |
| YU139- | GATGTTGTGATGACTCAGTCTCCTTCTTCCGTGTCTGCAT |
| A03 | CTGTAGGAGACAGAGTCACCATCACTTGTCGGGCGAGTCA |
| | GGGTATTAGCAGCTGGTTAGCCTGGTATCAGCAGAAACCA |
| | GGGAAAGCCCCTAAGCTCCTGATCTATGCTGCATCCAGTT |
| | TGCAAAGTGGGGTCCCATCAAGGTTCAGCGGCAGTGGATC |
| | TGGGACAGATTTCACACTCACCATCAGCAGCCTGCAGCCT |
| | GAAGATTTTGGAACTTACTATTGTCAACAGGCTAACAGTT |
| | TTCCGCTCACTTTCGGCGGAGGGACCAAGGTGGAGATCAA |
| | A (SEQ ID NO: 89) |
| YU139- | TCTTCTGAGCTGACTCAGGACCCTGCTGTGTCTGTGGCCT |
| B01 | TGGGACAGACAGTCAGGATCACATGCCAAGGAGACAGCCT |
| | CAGAAACTATTATGCAAGCTGGTACCAGCAGAAGCCAGGA |
| | CAGGCCCCTGTACTTGTCATCTATGGTAAAAACAACCGGC |
| | CCTCAGGGATCCCAGACCGATTCTCTGGCTCCAGCTCAGG |
| | AAACACAGCTTCCTTGACCATCAATGGGGCTCAGGCGGAA |
| | GATGAGGCTGACTATTACTGTAACTCCCGGGACAGCAGTG |
| | CTAAACATTGGGTGTTCGGCGGAGGGACCAAGCTGACCGT |
| | C (SEQ ID NO: 90) |
| YU139- | TCTTCTGAGCTGACTCAGGACCCTGCTGTGTCTGTGGCCT |
| C01 | TGGGACAGACAGTCAGGATCACGTGCCAAGGAGACGGCCT |
| | CAGAAACTATTTTGCAAGTTGGTACCAGCAGAAGCCAGGA |
| | CAGGCCCCTGTACTTGTCATCTATGGTAAAAACAACCGGC |
| | CCTCAGGGATCCCAGACCGATTCTCTGGCTCCAGCTCAGG |
| | AAACACAGCTTCCTTGAGAATCGCTGGGGCTCAGGCGGAA |
| | GATGAGGCTGACTATTACTGTAATTCCCGGGACGGCAGTG |
| | GTAAGCATCTGGTATTCGGCGGAGGGACCAAGTTGACCGT |
| | C (SEQ ID NO: 91) |
| YU139- | GACATCGTGATGACCCAGTCTCCATCCTCCCTGTCTGCAT |
| C02 | CTGTAGGAGACAGAGTCACCATCACTTGCCGGGCAAGTCA |
| | GAGCATTAGCAGCTATTTAAATTGGTATCAGCAGAAACCA |
| | GGGAAAGCCCCTAAGCTCCTGATCTATGCTGCATCCAGTT |
| | TGCAAAGTGGGGTCCCATCAAGGTTCAGTGGCAGTGGATC |
| | TGGGACAGATTTCACTCTCACCATCAGCAGTCTGCAACCT |
| | GAAGATTTTGCAACTTACTACTGTCAACAGAGTTACAGTA |
| | CCTGGACGTTCGGCCAAGGGACCAAGGTGGAAATCAAA |
| | (SEQ ID NO: 92) |
| YU139- | GACATCCAGATGACTCAGTCTCCTTCCACCCTGTCTGCAT |
| D02 | CTGTAGGAGACAGAGTCACCATCACTTGCCGGGCCAGTCA |
| | GAGTATTAGTAGGTGGTTGGCCTGGTATCAGCAGAAACCA |
| | GGGAAAGCCCCTAAGCTCCTGATCTATGATGCCTCCAGTT |
| | TGGAAAGTGGGGTCCCATCAAGGTTCAGCGGCAGTGGATC |
| | TGGGACAGAATTCACTCTCACCATCAGCAGCCTGCAGCCT |
| | GATGATTTTGCAACTTATTACTGTCAACAGAGTTACAGTA |
| | CCCCTCTTACTTTCGGCCCTGGGACCAAAGTGGATATCAA |
| | A (SEQ ID NO: 93) |
| YU139- | GAAACGACACTCACGCAGTCTCCAGGCACCCTGTCTTTGT |
| D03 | CTCCAGGGGAAAGAGCCACCCTCTCCTGCAGGGCCAGTCA |
| | GAGTGTTAGTGGCAACTACTTAGCCTGGTACCAGCAGAAA |
| | CCTGGCCAGCCTCCCAGGCTCCTCATCTTTGGTGCATCCA |
| | GCAGGGCCACTGGCATCCCAGACAGGTTCAGTGGCAGTGG |
| | GTCTGGGACAGACTTCACTCTCACCATCAGCAGACTGGAG |
| | CCTGAAGATTTTGGAGTGTATTACTGTCAGCAGTATGGTA |
| | GCTCACCAGGGACGTTCGGCCAAGGGACCAAGGTGGAAAT |
| | CAAA (SEQ ID NO: 94) |
| YU139- | GATGTTGTGATGACTCAGTCTCCATCCTCCCTGTCTGCAT |
| F02 | CTGTAGGAGACAGAGTCACCATCACTTGCCGGGCAAGTCA |
| | GAGCATTAGCAGCTATTTAAATTGGTATCAGCAGAAACCA |
| | GGGAAAGCCCCTAAGCTCCTGATCTATGCTGCATCCAGTT |
| | TGCAAAGTGGGGTCCCATCAAGGTTCAGTGGCAGTGGATC |
| | TGGGACAGATTTCACTCTCACCATCAGCAGTCTGCAACCT |
| | GAAGATTTTGCAACTTACTACTGTCAACAGAGTTACAGTA |
| | CCCCGCTCACTTTCGGCGGAGGGACCAAGGTGGAGATCAA |
| | A (SEQ ID NO: 95) |
| YU139- | CAGTCTGCCCTGACTCAGCCTCCCTCCGCGTCCGGGTCCC |
| G01 | CTGGACAGTCAGTCACCATCTCCTGCACTGGAACCAGCAG |
| | GTATGTTGGTGGTTATAACCATGTCTCCTGGTACCAGCAA |
| | CACCCAGGCAAAGCCCCCAAACTCATGATTTATGAGGTCA |
| | GTAATCGGCCCTCAGGAGTTCCTAATCGCTTCTCTGGCTC |
| | CAAGTCTGGCAACACGGCCTCCCTGACCATCTCTGGGCTC |
| | CAGGCTGAGGACGAGGCTGATTATTACTGCAGCTCATATA |
| | CAAGCAGCAACACTTGGGTGTTCGGCGGAGGGACCAAGCT |
| | GACCGTCCTA (SEQ ID NO: 96) |
| YU139- | GAAATTGTGCTGACTCAGTCTCCACTCTCCCTGCCCGTCA |
| G03 | CCCCTGGAGAGCCGGCCTCCATCTCCTGCAGGTCTAGTCA |
| | GAGCCTCCTGCATAGTAATGGAAACAACTATTTGAATTGG |
| | TACCTGCAGAAGCCAGGGCAGTCTCCACAGCTCCTGATCT |
| | ATTTGAGTTCTCGTCGGGCCTCCGGGGTCCCTGACAGGTT |
| | CAGTGGCAGTGGATCAGGCACAGATTTTACACTGAAAATT |
| | AGCAGAGTGGAGGCTGAGGATGTTGGGGTTTATTACTGCA |
| | TGCAAGGTCTACAAACTCCGTGGACGTTCGGCCAAGGGAC |
| | CAAGGTGGAAACCAAA (SEQ ID NO: 97) |
| YU139- | CAGGCAGTGCTGACTCAGCCACCCTCAGTGTCTGGGGCCC |
| H03 | CAGGGCAGAGGGTCACCATCTCCTGCACTGGGAGCAGCTC |
| | CAACATCGGGGCAGGTTATGATGTACACTGGTACCAGCAG |
| | CTTCCAGGAACAGCCCCCAAACTCCTCATCTATGGTAACA |
| | GCAATCGGCCCTCAGGGGTCCCTGACCGATTCTCTGGCTC |
| | CAAGTCTGGCACCTCAGCCCCCCTGGCCATCACTGGGCTC |
| | CAGGCTGAGGACGAGGCTGATTATTACTGCCAGTCTTATG |
| | ACAGCAGCCTGAGTACGGTATTCGGCGGAGGGACCAAGCT |
| | GACCGTCCTA (SEQ ID NO: 98) |

TABLE 8
Constant region amino acid sequences

| Constant | |
|---|---|
| region | AA |
| Human | ASTKGPSVFPLAPCSRSTSESTAALGCLVKDYFPEP |
| IgG4 | VTVSWNSGALTSGVHTFPAVLQSSGLYSLSSVVTVP |
| heavy | SSSLGTKTYTCNVDHKPSNTKVDKRVESKYGPPCPS |
| chain | CPAPEFLGGPSVFLFPPKPKDTLMISRTPEVTCVVV |
| P01861.1 | DVSQEDPEVQFNWYVDGVEVHNAKTKPREEQFNSTY |
| | RVVSVLTVLHQDWLNGKEYKCKVSNKGLPSSIEKTI |
| | SKAKGQPREPQVYTLPPSQEEMTKNQVSLTCLVKGF |
| | YPSDIAVEWESNGQPENNYKTTPPVLDSDGSFFLYS |
| | RLTVDKSRWQEGNVFSCSVMHEALHNHYTQKSLSLS |
| | LGK (SEQ ID NO: 99) |
| Human | ASTKGPSVFPLAPCSRSTSESTAALGCLVKDYFPEP |
| IgG2 | VTVSWNSGALTSGVHTFPAVLQSSGLYSLSSVVTVP |
| heavy | SSNFGTQTYTCNVDHKPSNTKVDKTVERKCCVECPP |
| chain | CPAPPVAGPSVFLFPPKPKDTLMISRTPEVTCVVVD |
| P01859 | VSHEDPEVQFNWYVDGVEVHNAKTKPREEQFNSTFR |
| | VVSVLTVVHQDWLNGKEYKCKVSNKGLPAPIEKTIS |
| | KTKGQPREPQVYTLPPSREEMTKNQVSLTCLVKGFY |
| | PSDISVEWESNGQPENNYKTTPPMLDSDGSFFLYSK |
| | LTVDKSRWQQGNVFSCSVMHEALHNHYTQKSLSLSP |
| | GK (SEQ ID NO: 100) |
| Human | GQPKANPTVTLFPPSSEELQANKATLVCLISDFYPG |
| light | AVTVAWKADGSPVKAGVETTKPSKQSNNKYAASSYL |
| chain, | SLTPEQWKSHRSYSCQVTHEGSTVEKTVAPTECS |
| lambda | (SEQ ID NO: 101) |
| 1 | |
| P0CG04 | |
| Human | GQPKAAPSVTLFPPSSEELQANKATLVCLISDFYPG |
| light | AVTVAWKADSSPVKAGVETTTPSKQSNNKYAASSYL |
| chain, | SLTPEQWKSHRSYSCQVTHEGSTVEKTVAPTECS |
| lambda | (SEQ ID NO: 102) |
| 2 | |
| P0DOY2 | |
| Human | RTVAAPSVFIFPPSDEQLKSGTASVVCLLNNFYPRE |
| light | AKVQWKVDNALQSGNSQESVTEQDSKDSTYSLSSTL |
| chain, | TLSKADYEKHKVYACEVTHQGLSSPVTKSFNRGEC |
| kappa | (SEQ ID NO: 103) |
| P01834 | |

### Example 2

A Commercially Available Anti-IGFBP3 Monoclonal Antibody does not Affect IGFBP3/TMEM219 Binding

A commercially available anti-IGFBP3 antibody was tested for its ability to displace the binding of ecto-TMEM219 to IGFBP3. Displacement of binding would indicate both (i) the ability of the antibody to inhibit binding of IGFBP3 to the native TMEM219 receptor, and (ii) the ability of the antibody to mimic the neutralizing activities of the ecto-TMEM219 ligand trap fusion protein.

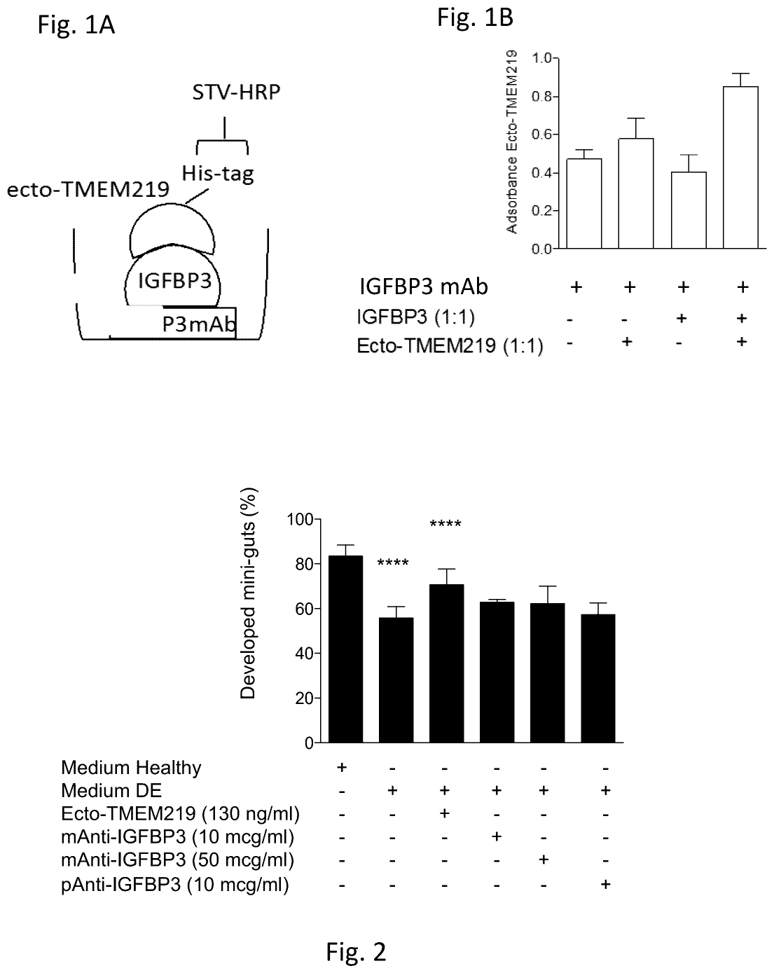

As shown in FIG. 1A, we coated the plate with the commercial anti-IGFBP3 monoclonal antibody and added a pre-formed complex of IGFBP3 and Ecto-TMEM219, formed by pre-incubating IGFBP3 and Ecto-TMEM219 for 30 minutes. We then assessed the signal of Ecto-TMEM219 by using an anti-His tag and an anti-His-tag Streptavidin-HRP to detect whether Ecto-TMEM219 was still bound to the complex (high signal) or was displaced and washed away (low signal). Results are shown in FIG. 1B.

As shown in FIG. 1B, the commercial anti-IGFBP3 mAb is able to bind the IGFBP3-Ecto-TMEM219 complex, thus suggesting that it did not displace the IGFBP3-Ecto-TMEM219 binding. A monoclonal anti-IGFBP3 antibody (LSBio) was evaluated in a competitive ELISA binding assay (FIG. 1B).

### Commercially Available Anti-IGFBP3 Antibodies do not Rescue IGFBP3-Mediated Damage of Mini-Guts in Different Disease Models.

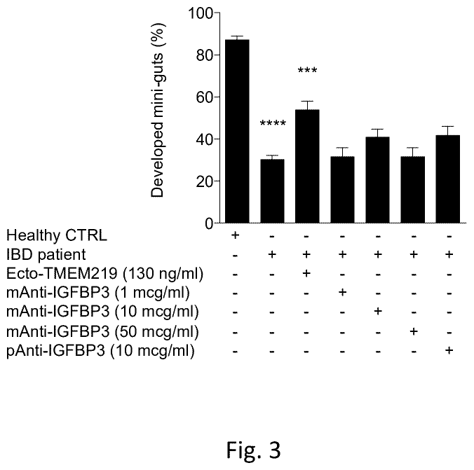

In order to assess whether commercially available antibodies, both monoclonal and polyclonal, could mimic the ability of the ecto-TMEM219 ligand trap to rescue mini-gut growth in intestinal stem cell (ISC) injury disease conditions though preventing the binding of IGFBP3 to TMEM219, they were further tested in vitro in the mini-gut assay. Mini-guts were generated from healthy controls as already described (4, 18). As shown in the left-most 3 bars in FIG. 2, incubation in the presence of medium from patients with diabetic enteropathy (“Medium DE”) significantly reduced formation of mini-guts as compared to incubation in medium from healthy individuations (“Medium Health”); further addition of Ecto-TMEM219 at 130 ng/ml restored mini-gut growth in the presence of Medium DE.

Monoclonal and polyclonal antibodies were tested at three different concentrations in the presence of medium from individuals with diabetic enteropathy (“Medium DE”).

Neither commercially available monoclonal nor polyclonal anti-IGFBP3 antibodies significantly improved the development of mini-guts in the presence of medium from individuals with diabetic enteropathy. None of the tested antibodies mimicked the ability of ecto-TMEM219 to rescue mini-gut development, suggesting that commercial anti-IGFBP3 antibodies would not be able to prevent ISC damage in disease conditions such as diabetic enteropathy; consistent with the data from Example 1, we infer that this lack of functional activity can be attributed to their inability to prevent binding of IGFBP3 to TMEM219 binding.

This highlights the need for other anti-IGFBP3 antibodies that selectively prevent the IGFBP3-TMEM219 interaction.

Antibodies were also tested in mini-gut assays using crypts obtained from patients with established Crohn's disease (an inflammatory bowel disease, IBD) undergoing surgery for disease complications (strictures, fistulae). This model is highly clinically relevant as it recapitulates in vitro the major features of the disease condition in patients.

As shown in FIG. 3, we observed that mini-guts of IBD patients failed to fully grow and develop, and that ecto-TMEM219 was able to rescue mini-guts growth and re-establish organoids development. By contrast, commercially available antibodies tested at different molecular ratio did not exert any effect.

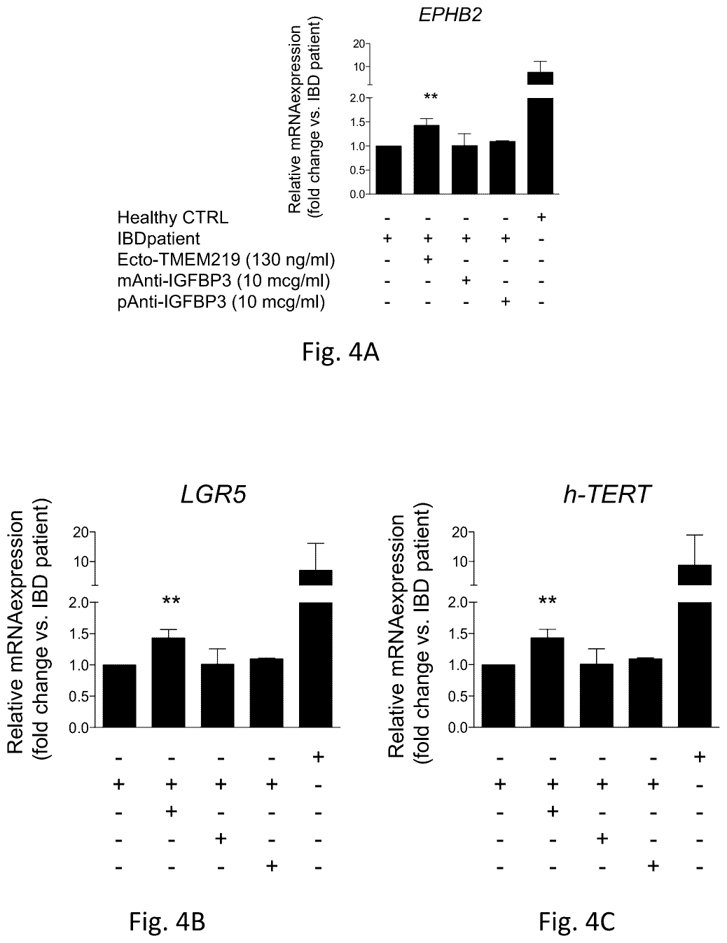

Next, we showed that the expression of the ISC markers EphB2, LGR5, and h-TERT in IBD patients, detected by RT-PCR was not rescued by the addition of commercially available anti-IGFBP3 Abs, in contrast to Ecto-TMEM219 (FIGS. 4A, B and C). These results confirm the poor effects of the commercial anti-IGFBP3 monoclonal and polyclonal antibodies in targeting ISCs function (mini-guts development) and in rescuing

ISC markers expression. This also emphasizes that different disease conditions (e.g. diabetic enteropathy, IBD) in which ISCs are damaged due to the activation of IGFBP3-TMEM219 signaling, may benefit from a strategy that selectively targets it.

### Example 3

Novel Anti-IGFBP3 mAbs Inhibit IGFBP3-TMEM219 Binding

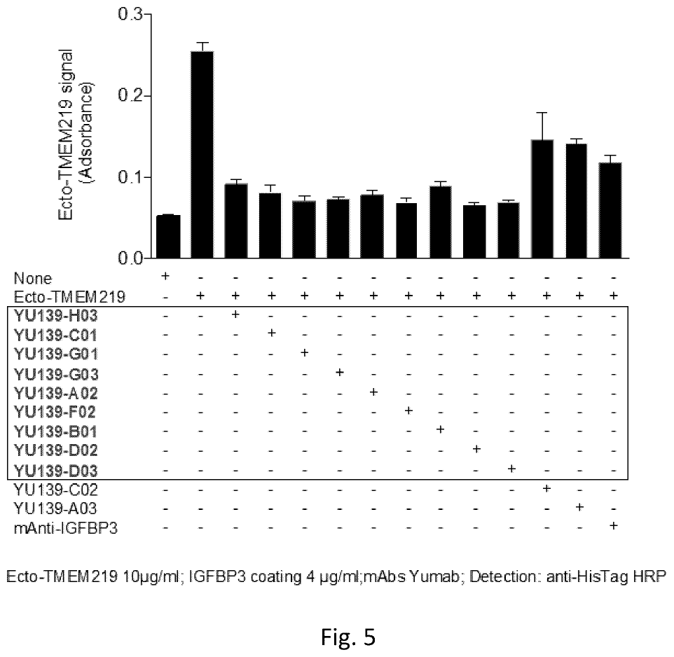

As discussed in Example 1, a set of anti-IGFBP3 monoclonal antibodies was selected by phage display. These novel antibodies were then screened for their ability to compete with ecto-TMEM for the binding to IGFBP3 using a competitive ELISA binding assay. IGFBP3 (coating), ecto-TMEM219 and the anti-IGFBP3 antibodies selected by phage display were all used in a 1:1 molecular ratio. As shown in FIG. 5, the novel antibodies were capable of inhibiting IGFBP3-ecto-TMEM219 binding, although two of them to a lesser extent. In this assay, the commercially available monoclonal anti-IGFBP3 antibody was poor at inhibiting IGFBP3-ecto-TMEM219 binding.

This demonstrates that the majority of the newly discovered Anti-IGFBP3 mAbs may prevent the IGFBP3-TMEM219 binding to a higher rate as compared to the commercially available anti-IGFBP3 antibody.

### Novel Anti-IGFBP3 Antibodies Rescue IGFBP3-Damage in the Mini-Guts Assay

The 11 newly discovered monoclonal antibodies were also tested in the mini-gut assay. Mini-guts were generated from crypts obtained from healthy controls and cultured for 8 days in presence of IGFBP3 and treated with either ecto-TMEM219 or the newly discovered anti-IGFBP3 mAbs All factors were included after the first day of culture at a 1:1 ratio (mAbs/ecto-TMEM219:IGFBP3).

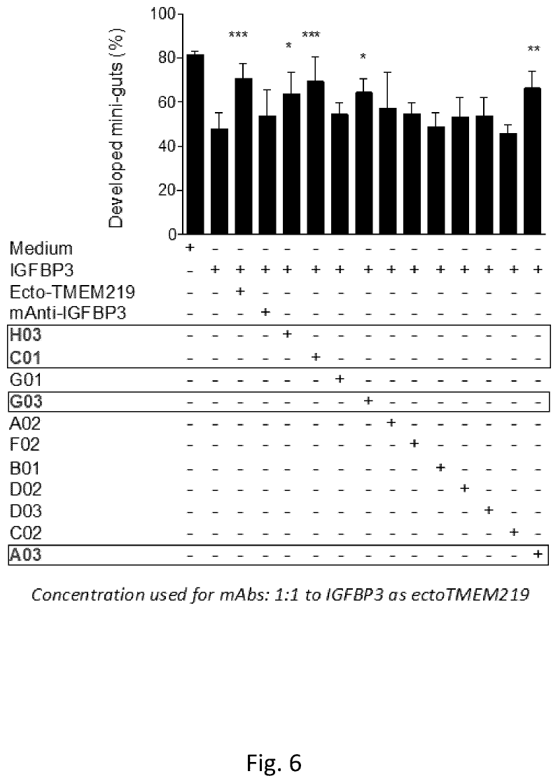

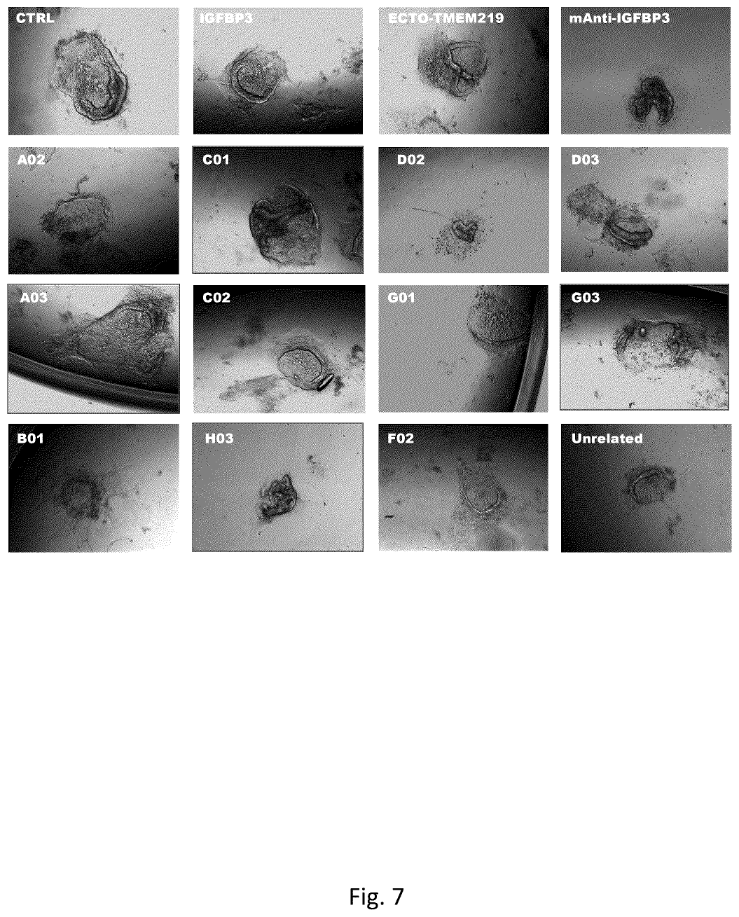

As shown in FIGS. 6 and 7, ecto-TMEM219 rescues the negative effects of IGFBP3 on self-renewal ability (% development) and morphology of large crypt organoids. Further, antibodies YU139-C01, YU139-A03, YU139-G03, and YU139-H03 also rescue the negative effects of IGFBP3 on self-renewal ability (% development) and morphology (absence of crypts domain, generation of small spheroids) of large crypt organoids (FIGS. 6 and 7), similarly to ecto-TMEM219.

### Novel Monoclonal Anti-IGFBP3 Antibodies Rescue IGFBP3-Damage on ISCs Markers

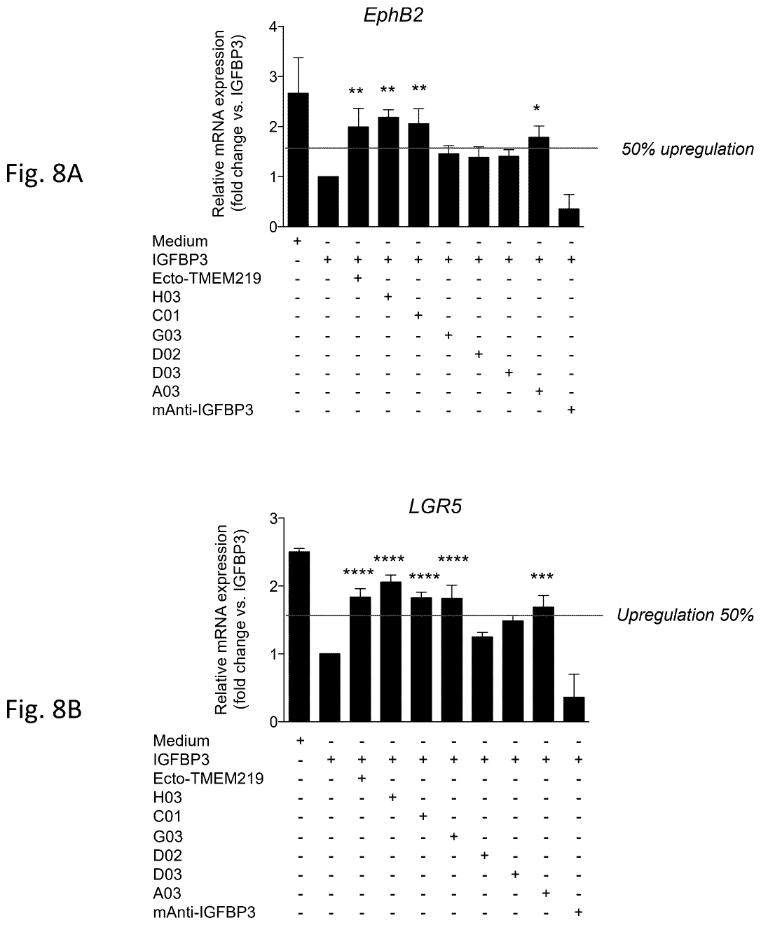

The newly discovered anti-IGFBP3 mAbs which were shown to be effective in promoting mini-guts development upon IGFBP3 exposure were also able to significantly upregulate the expression of ISCs markers EphB2 (FIG. 8A) and LGR5 (FIG. 8B) in presence of IGFBP3 by at least 50% compared to samples treated with only IGFBP3. This result is similar to that observed with ecto-TMEM219.

### Novel Monoclonal Anti-IGFBP3 Antibodies Inhibit IGFBP 3-Mediated Overexpression of Caspase 8

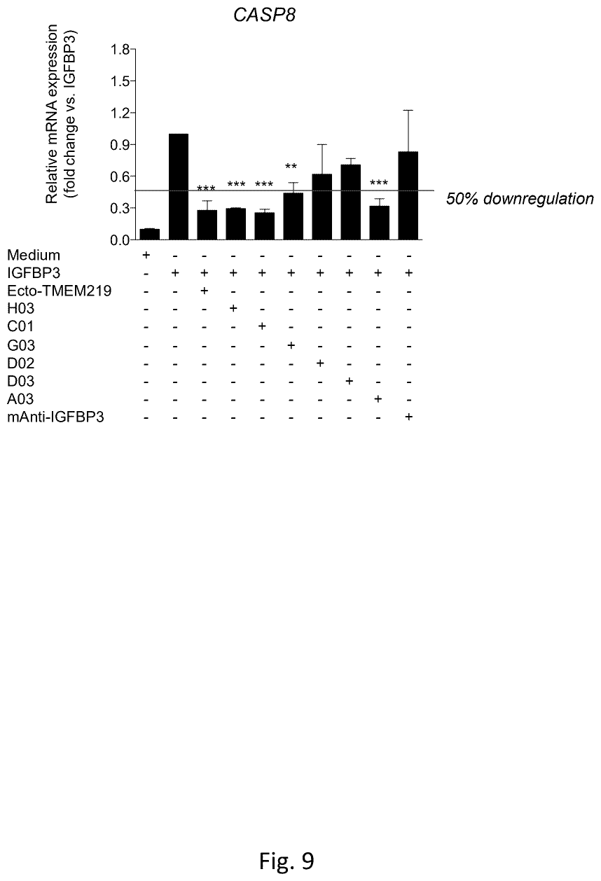

IGFBP3-detrimental effects on ISCs are Caspase-8 mediated. Interestingly, newly discovered anti-IGFBP3 mAbs were able to inhibit the caspase-8 upregulation induced by IGFBP3 treatment by at least 50% when compared to samples treated only with IGFBP3 (FIG. 9).

These results suggest that the discovered anti-IGFBP3 mAbs exert a protective effect on ISCs pool by blocking the IGFBP3/TMEM219 Caspase-8-mediated apoptotic injury.

### The Newly Discovered Anti-IGFBP3 Antibodies Rescue Mini-Guts Growth in Disease Models.

In order to confirm that the newly discovered monoclonal anti-IGFBP3 antibodies prevent the detrimental effects of IGFBP3 on TMEM219-expressing intestinal stem cells, the inventors further tested them in vitro in the mini-gut assay in an established model of DE.

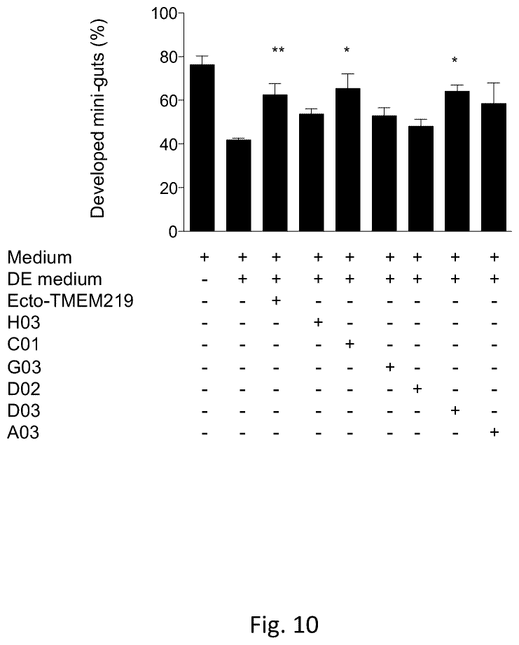

The novel anti-IGFBP3 mAbs significantly improved the development of mini-guts in DE of at least 20%, similarly to Ecto-TMEM219 treatment (FIG. 10).

This highlights that anti-IGFBP3 mAbs of the invention, selected both for binding to IGFBP3 and screened for their ability to competitively inhibit ecto-TMEM binding to IGFBP3 are capable of rescuing ISCs function and preserve ISCs pool from IGFBP3-detrimental effects.

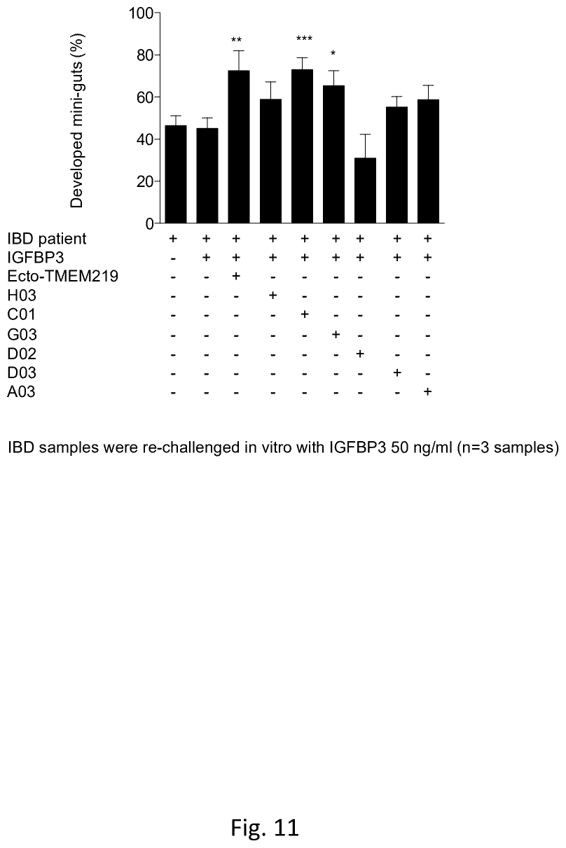

The inventors also demonstrated that mini-guts growth was significantly recovered, by at least 20%, when treating mini-guts obtained from IBD patients with the newly discovered anti-IGFBP3 mAbs. The effects obtained with the novel anti-IGFBP3 mAbs were similar to that exerted by ecto-TMEM219 (FIG. 11).

These results show a potential benefit in preserving the ISCs pool and function also in this disease model.

The Newly Discovered Human Monoclonal Anti-IGFBP3 Antibodies Bind a Different Region of the Antigen from the Commercial Mouse Monoclonal Anti-IGFBP3

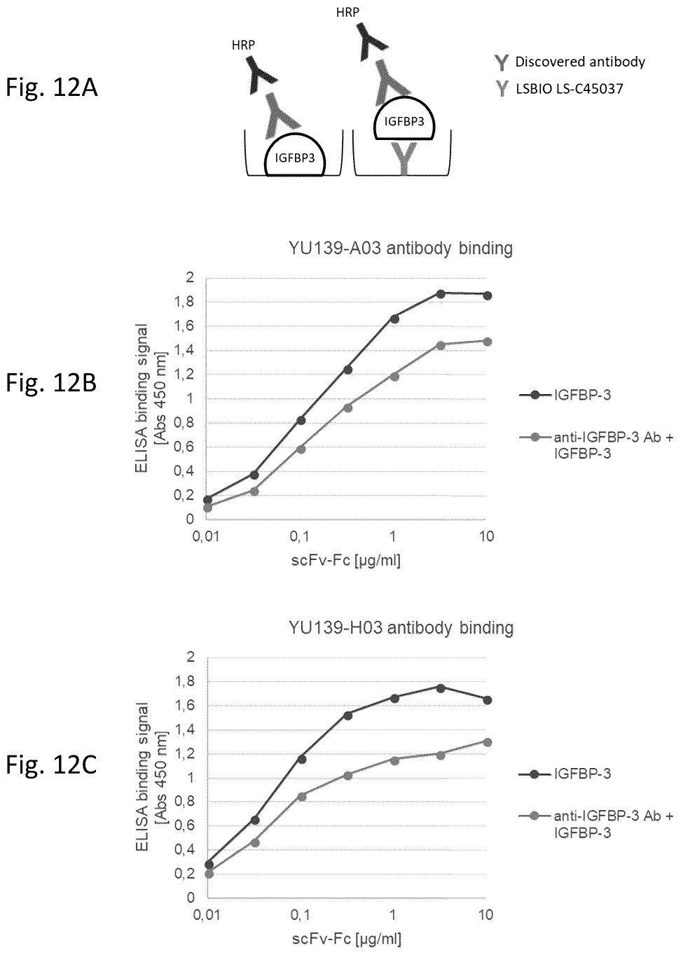

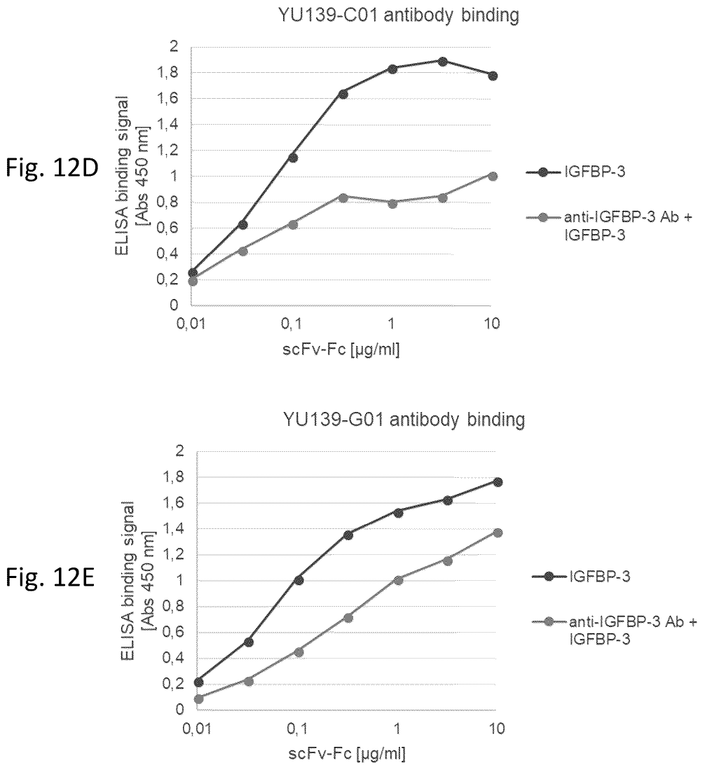

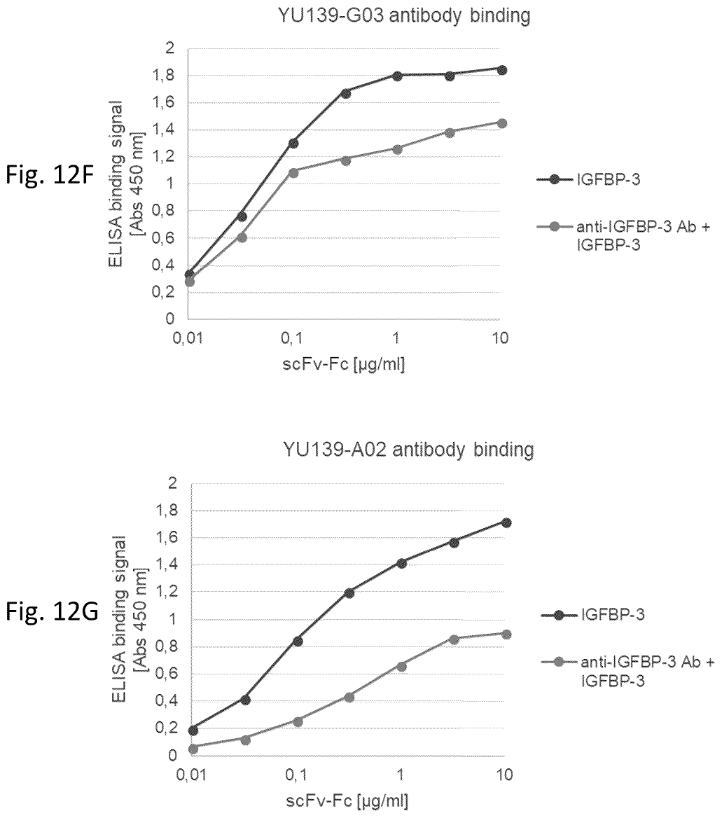

An epitope binning experiment was performed to check whether commercial antibody LSBIO LS-C45037 (clone 83.8F9) and the newly developed anti-IGFBP3 antibodies bind to the same epitope region of IGFBP3. Briefly, a sandwich ELISA was performed coating the multiwell plate with the prior art antibody (FIG. 12A). All of the novel anti-IGFBP3 monoclonal antibodies selected by phage display were able to bind to IGFBP3 in the presence of commercial antibody LSBIO LS-C45037 (clone 83.8F9).

This result suggests that the epitope on IGFBP3 bound by the newly discovered antibodies is different from that of commercially available antibody (FIG. 12B-12G).

### Example 4

### Newly Generated Monoclonal Anti-IGFBP3 Antibodies do not Displace IGF-I-IGFBP3 Binding

### Competitive ELISA Binding Assay

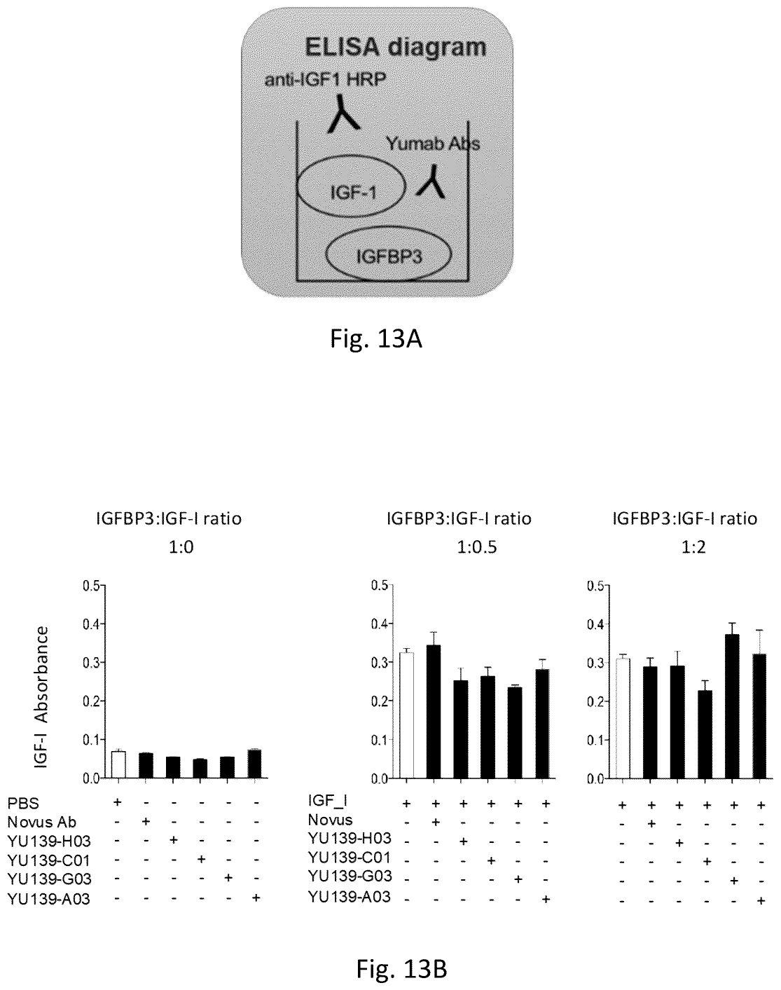

Newly generated anti-IGFBP3 mAbs were tested for their ability to displace the IGF-I-IGFBP3 interaction by using a competitive ELISA in vitro (FIG. 13A). Briefly, plate was coated with IGFBP3 (Life Technologies) 4 μg/ml. IGF-I (R&D, 291-G1-01M) was added in a dose dependent ratio with IGFBP3 (0:1, 0.5:1 and 2:1). The newly generated anti-IGFBP3 mAbs (Yumab) of the invention were added at a concentration of 1:1 as compared to IGFBP3. IGFBP3-IGF-I binding was revealed by using anti-IGF-I HRP conjugated antibody.

The following reagents were used to screen the anti-IGFBP3 Antibodies: Recombinant Human IGFBP-3 (0,223 mg/ml R&D System, 675-B3-025), IGF-I (R&D, 291-G1-01M), anti-IGFBP3 mAbs (Yumab), mouse monoclonal anti-IGFBP3 (Novus Biological, NBP2-12364), anti-IGF-I HRP (LSBio, C547140), bovine serum albumin (BSA, Sigma A7906), Tween 20 (TW), ELISA colorimetric TMB reagent (HRP substrate, Item H Sigma, RABTMB3), ELISA STOP solution (Item I, Sigma, RABSTOP3), blocking reagent solution (3% BSA in PBS) and a diluent solution (0,5% BSA, 0.05% Tw in PBS).

Microplate (Thermofisher, Electron Corporation, 2801) was coated with 50 μl/well of 4 μg/ml rhIGFBP3 dissolved in PBS or PBS alone (no coating). Plate was incubated 90 minutes at 37° C. and washed with PBS (300 μl/well) and incubated with the blocking reagent (200 μl/well) 2 hours at room temperature. Samples were then diluted in the diluent solution (50 μl/well) and added to the plate as following: diluent solution (none), IGF-I at different ratios with rhlGFBP3 (0:1, 0.5:1 and 2:1) and anti-IGFBP3 mAbs at 10 μg/ml. After washing steps, plate was then incubated at room temperature for 1 hour with anti-IGF-I HRP diluted in Diluent solution (50 μl/well). ELISA plate was then read after adding visualization solution at ELISA reader.

As shown in FIG. 13B, newly generated anti-IGFBP3 mAbs did not reduce significatively the absorbance of IGF-I in the assay, indicating that the mAbs are unable to fully displace the IGF-I-IGFBP3 binding at low or high concentration of IGF-I. Overall, the present results confirm that when IGF-I is available, IGFBP3 preferentially binds to it and that newly generated anti-IGFBP3 mAbs do not interfere with the IGFBP3-IGF-I binding in vitro.

In other words, when IGF1 is available, IGFBP3 preferentially binds to IGFF1 and the newly generated mAbs do not interfere with the IGF1-IGFBP3 binding.

### Newly Generated Monoclonal Anti-IGFBP3 Antibodies Rescue IGFBP3-Damage in Murine Mini-Guts

### Crypts Isolation and Murine Mini-Guts Development

In order to confirm that the present invention antibodies have a similar tissue cross-reactivity profile in murine tissue in respect to the human tissues, the inventors further tested the monoclonal anti-IGFBP3 antibodies of the invention in the in vitro mini-gut assay in murine crypts. Crypts were obtained from C57BL/6J mice (632C57BL/6J Charles River Laboratories, Lyon, France). Briefly, the colon was cut into 2-4 mm pieces with scissors and fragments were washed in 30 ml of ice-cold PBS and then incubated with 20 mM EDTA-PBS at 37° C. Then, fragments were treated trypsin/DNAse solution to obtain crypts. After this step, vigorous shaking of the sample yielded supernatants enriched in colonic crypts. Crypts were mixed with Matrigel and plated on pre-warmed culture dishes. After solidification of matrigel (10-15 min at 37° C.), crypts were overlaid with culture medium (ADF, 10 mM HEPES, N-2, B27 without retinoic acid, 10 UM Y-27632, 1 μM JAG1 peptide (Anaspec, Fremont, CA, USA), 1 μg/ml R-Spondin 1, 50 ng/ml EGF (Invitrogen), and 100 ng/ml Noggin (Peprotech, Rocky Hill, NJ, USA), and medium was changed every other day until day 8.

Isolated crypts were cultured to generate large crypts organoids namely mini-guts for 8 days in the presence of IGFBP3 with/without ecto-TMEM219. Newly generated anti-IGFBP3 mAbs were added at day 0 at a ratio of 1:1 (mAbs/ecto-TMEM219: IGFBP3). Miniguts development was calculated as a percentage of organoids growth after 8 days as compared to the plated isolated crypts (D′Addio F et al. Cell Stem Cell 2015 Oct. 1; 17 (4): 486-498).

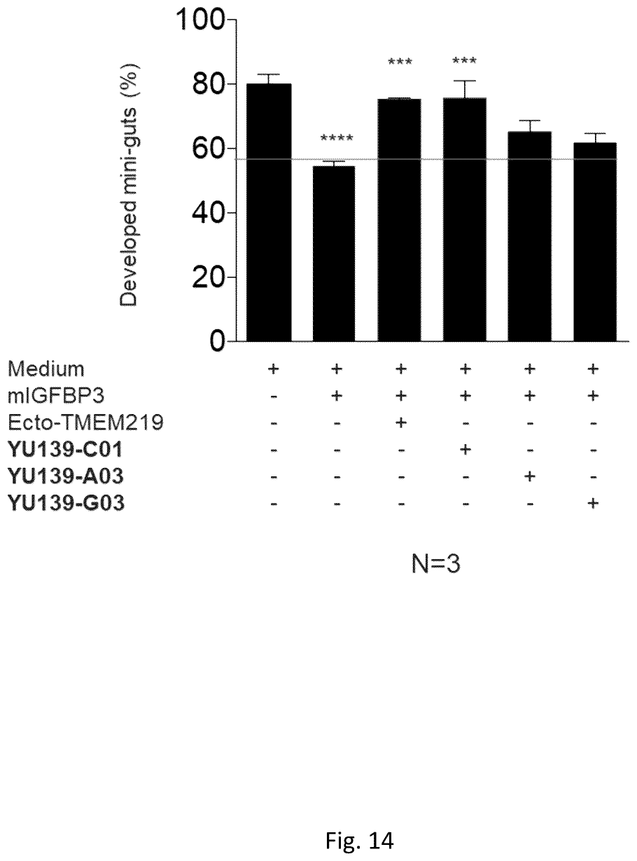

As shown in FIG. 14, antibodies YU139-C01, YU139-A03, YU139-G03, and YU139-H03 rescue the negative effects of IGFBP3 on murine mini-gut self-renewal ability (% development) and morphology (absence of crypts domain, generation of small spheroids) of large crypt organoids, similarly to what is observed for ecto-TMEM219.

### Affinity Measurement

### Octet BLI-Based Analysis

To assess the binding affinity in more detail, affinity measurements were performed using Biomolecular Interaction Analysis performed by an Octet instrument (BLItz System), which is a Biolayer Interferometry (BLI) platform. To establish the assay, the target monoclonal antibody (30 μg/ml in 1% BSA-PBST) was immobilized via Fc on the via AHC biosensor and the interaction with the antigen human IGFBP3 (R&D, cat n° 675 B3) and mouse IGFBP3 (Creative Biomart, cat n° igfbp3 829M) at different concentration was measured.

The affinity measurement of YU139-C01, YU139-H03, YU139-G03 and YU139-A03 for the target human and murine IGFBP3 are reported in the Table 9.

TABLE 9
Affinity of YU139-C01, YU139-H03, YU139-G03 and
YU139-A03 for the target human and murine IGFBP3.

| Molecule | EctoTMEM- | YU139- | YU139- | YU139- |
|---|---|---|---|---|
| id | His | C01 | H03 | A03 |
| hIGFBP-3 | 3.5 × 10⁻⁸ | 1.69 × 10⁻⁹ M | 4.0 × 10⁻⁶ | 3.28 × 10⁻⁷ |
| KD (M) | | | | |
| mIGFBP-3 | 6.0 × 10⁻⁸ | 3.42 × 10⁻⁷ | >10⁻⁶ | 7.35 × 10⁻⁷ |
| KD (M) | | | | |

Newly generated anti-IGFBP3 mAbs show good human antigen binding affinity with KD below 4×10⁻⁶ M. The antibodies also show murine cross reactivity. This data confirmed that mice can be considered a relevant animal species for testing of the monoclonal antibodies during preclinical development.

Anti-IGFBP3 mAbs Efficacy in IBD Mouse Model Following Intraperitoneal (IP) Administration

The model of colitis induced by Dextran sulfate sodium (DSS) in C57BL/6J mice is a validated animal model to evaluate and also to confirm the anti-inflammatory properties of drugs in IBD. DSS (oral administration in the drinking water) induces prominent diarrhea followed by inflammation. This model is well characterized, reliable, reproducible and admitted by regulatory authorities in IBD with no mortality. This study was performed in C57BL/6J mice. Indeed, in this particular genetic background, mice develop acute colitis when analyzed 3 days after the last DSS administration or a chronic-like inflammation when analyzed 7 days after the last DSS administration.

### Animals

Male C57BL/6J mice were supplied by Charles River laboratories, l'Arbresle, France. The mice were housed at 20±5° C. and provided with water and food ad libitum. All experimental protocols were performed in accredited facilities at Institut Pasteur from Lille according to governmental guidelines.

### Establishment of DSS-Induced Mice Colitis Model and Treatment

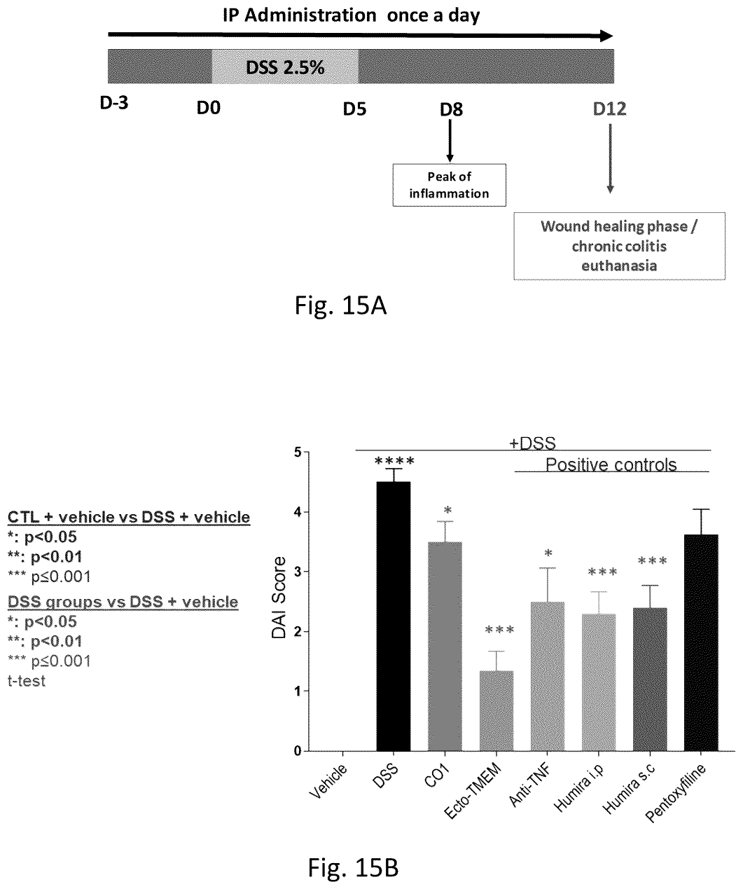

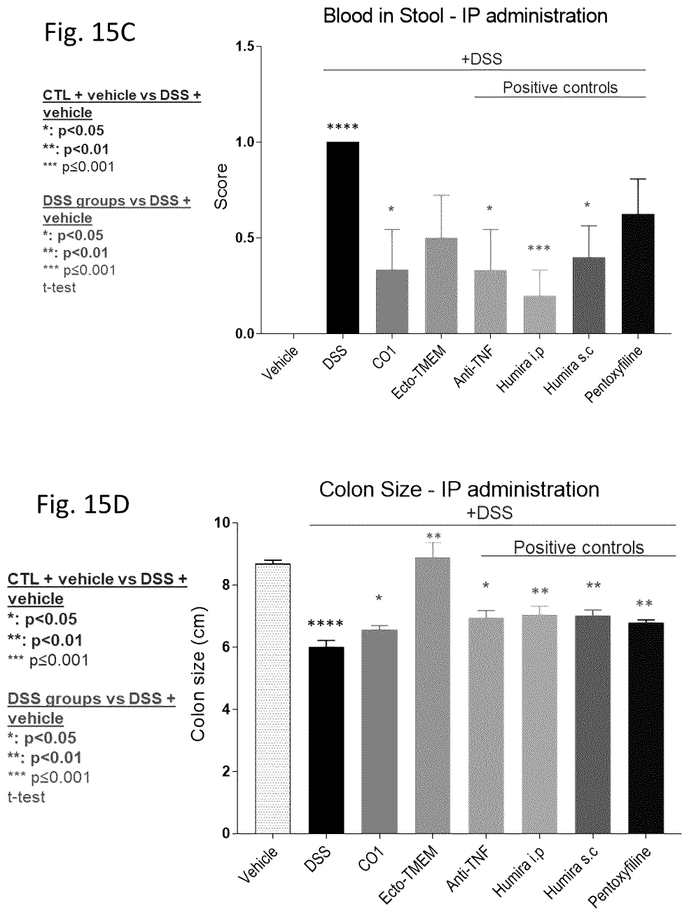

Acute colitis was induced by feeding mice with 2.5% (w/v) DSS (45 kD; TDB Consultancy AB, Uppsala, Sweden, Batch number DB001-41) dissolved in drinking water for 5 days. The mice were randomly divided into eight groups: control group; DSS+vehicle; DSS+Yu139-CO1 0.6 mg/mouse, DSS+Ecto-TMEM 0.1 mg/mouse, DSS+mouse anti-TNFα 0.1 mg/mouse (Ozyme, B270358), DSS+Pentoxifylline (Sigma P1784, BCBQ05 15), DSS+Humira (Abbvie, 1108722) ip, DSS+Humira sc. To assess the preventive therapeutic effects of anti-IGFBP3 mAb on DSS-induced acute colitis in C57BL/6J mice, the mice were treated daily by intraperitoneal administration with indicated dose of Yu139-CO1 starting 3 days before colitis induction and were performed until euthanasia occurring 7 days after the last DSS administration. The experimental timelines of the animal model are described in FIG. 15A.

The therapeutic properties of the biologics were compared to those induced by positive control groups, mouse anti-TNFα antibody, Pentoxifylline and Humira (Taghipour N. et al Gastroenterol Hepatol Bed Bench 2016; 9(1):45-52).

As shown on FIG. 15B, in the group of DSS mice receiving the vehicle, 7 days after the last administration of DSS, the DAI score was significantly increased compared to healthy control group (group receiving vehicle only) indicating the severity of the colitis. A significant decrease in the DAI score was recorded in colitic mice receiving Yu139-CO1, Ecto-TMEM219, Humira and mouse anti-TNFα antibody compared to DSS mice receiving only the vehicle. This result indicates an anti-inflammatory effect of the newly generated anti-IGFBP3 mAbs.

As shown on FIG. 15C, a significant decrease of the presence of occult blood was recorded in DSS mice receiving a treatment with Yu139-CO1, Humira (the biologic benchmark used in the treatment of IBD patients, used to treat rheumatoid arthritis, psoriatic arthritis, ankylosing spondylitis, Crohn's disease, ulcerative colitis, psoriasis, hidradenitis suppurativa, uveitis, and juvenile idiopathic arthritis) and mouse anti-TNFα antibody, compared to colitic mice receiving the vehicle.

FIG. 15D shows a significant increase of the size of the colon in colitic mice receiving Yu-139-CO1, and the 4 positive controls, indicating therapeutic properties of the newly generated anti-IGFBP3 mAbs.

### Clinical Scoring

In all groups, mice weight, stool consistency and blood in stool were recorded daily. The disease activity index (DAI) scores were based on changes in body weight, consistency of stool, and hemoccult bleeding according to a standard scoring system. These parameters were assessed on a scale as described in the Table 10. The DAI data are presented as an average score of these parameters taken daily. Animals were sacrificed by cervical dislocation under anesthesia. At euthanasia, colons were carefully dissected, and its weight and size were measured. At euthanasia, the presence of Occult Blood (OB) is recorded using the hemoccult method.

TABLE 10
Parameters assessed for DAI score

| | Parameters | Scores |
|---|---|---|
| Disease | Weight loss | 0: no; 1: <10%; 2: ≥10% |
| Activity Index | Stool consistency | 0: regular; 1: soft; 2: diarrhea |
| (DAI) (0-5) | Blood occurrence | 0: absence; 1: presence |

### Statistical Analysis

All comparisons were analysed using the Permutation Test for two independent samples. Statistics have been calculated using the GraphPad Prism version 7.0 (GraphPad Software, San Diego, CA). Differences were considered statistically significant if the p value was <0.05.

Anti-IGFBP3 mAbs Efficacy in T1D Mouse Model Following Intraperitoneal (IP) Administration

### Animals

Female non-obese diabetic (NOD) mice (10 weeks old) were obtained from the Jackson Laboratory, Bar Harbor, Maine (JAX stock #001976). All mice were cared for and used in accordance with institutional guidelines approved by the Jackson Laboratory Institutional Animal Care and Use Committee.

### Diabetes Monitoring and Treatment

Overt diabetes (the most advanced stage, characterized by elevated fasting blood glucose concentration and classical symptoms) was defined as blood glucose levels above 300 mg/dL. Blood glucose was measured using the Breeze2 (Bayer S.p.A) blood glucose meter.

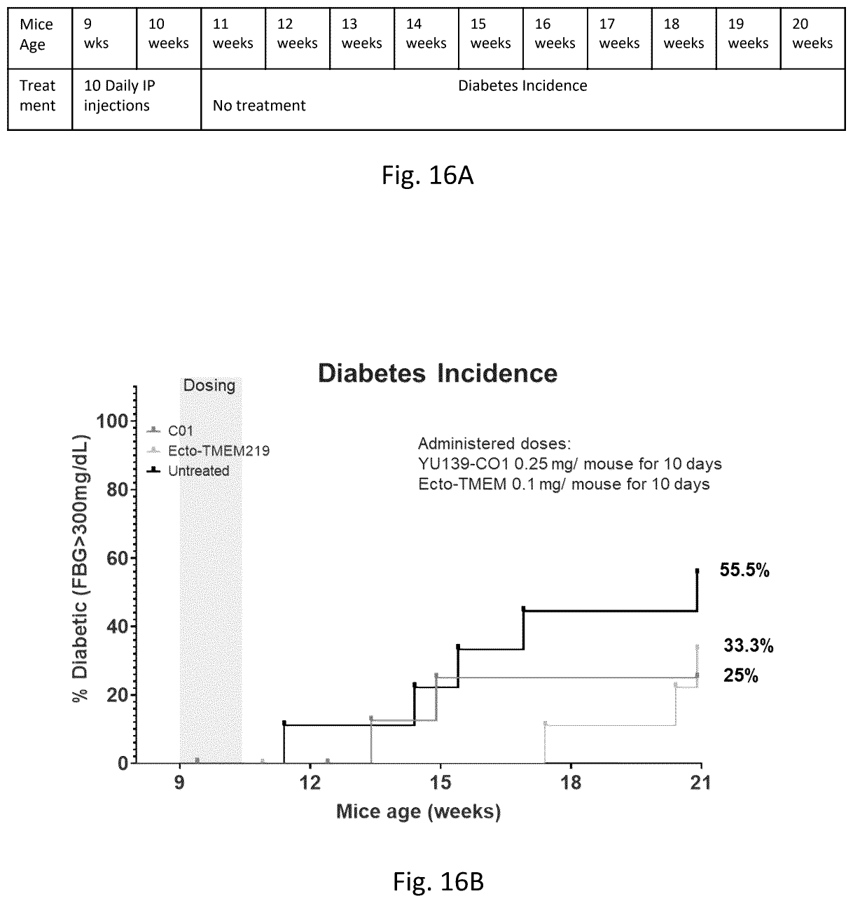

The experiment was randomly divided into three groups: control group; Yu139-CO1 0.25 mg/mouse and Ecto-TMEM 0.1 mg/mouse. To determine whether the anti-IGFBP3 mAb prevents diabetes in NOD mice, animals were treated daily for 10 days by intraperitoneal administration with the indicated dose of Yu139-CO1. The experimental timelines are described in FIG. 16A.

### Statistical Analysis

Data are presented as mean and standard deviation (SD) and were tested for normal distribution with the Kolmogorov-Smirnov test and for homogeneity of variances with Levene's test. The statistical significance of differences was tested with two-tailed t-test and the chi-square (χ2) tests. Significance between the two groups was determined by two-tailed unpaired Student's t test. For multiple comparisons, the 1-way ANOVA test with Bonferroni correction was employed. Diabetes incidence among different groups was analyzed with the log-rank (Mantel-Cox) test. Statistical analysis was conducted using GraphPad Prism version 7.0 (GraphPad Software, La Jolla, CA). All statistical tests were performed at the 5% significance level.

As shown in FIG. 16B, the inventors assessed if 10 days of newly generated anti-IGFBP3 mAb administration could be effective to prevent clinical diabetes in NOD mice. Remarkably, Anti-IGFBP3 mAbs given by the intraperitoneal (IP) route keep blood glucose under control and are effective in preventing Diabetes in T1D NOD mouse model. This treatment induced diabetes prevention in 75% of the mice with 21 weeks of age.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

### REFERENCES

1. Baxter R C. J Cell Commun Signal. 2013; 7(3):179-89.
2. Oh Y, et al., Prog Growth Factor Res. 1995; 6(2-4):503-12.
3. Ingermann A R, et al. IThe Journal of biological chemistry. 2010; 285(39):30233-46.
4. D′Addio F, et al. Cell stem cell. 2015; 17(4):486-98.
5 Brennand K, and Melton D. J. of cellular and molecular medicine. 2009; 13(3):472-87.
6. Yi P, Park J S, and Melton D A. Cell. 2014; 159(3):467-8.
7. Ben Nasr M, et al., Pharmacological research: the official journal of the Italian Pharmacological Society. 2015; 98:31-8.
8. Keenan H A, et al. Diabetes. 2010; 59(11):2846-53.
9. Meier J J, et al., Diabetologia. 2005; 48(11):2221-8.
10. Atkinson M A, et al., Diabetes care. 2015; 38(6):979-88.
11. Nguyen K H, et al., Endocrinology. 2011; 152(6):2184-96.
12. Yakar S, et al., FASEB J. 2009; 23(3):709-19.
13. Drogan D, et al., Am J Epidemiol. 2016; 183(6):553-60.
14. Peet A, et al., Eur J Endocrinol. 2015; 173(2):129-37.
15. Kaplan G G. Nat Rev Gastroenterol Hepatol. 2015; 12(12):720-7.
16. Cui S, and Chang P Y. World J Gastroenterol. 2016; 22(31):7099-110.
17. Yancu D, et al., J Gastroenterol Hepatol. 2017; 32(1):146-53.
18. Jung P, et al., Nature Medecine 2011; 17:1225-1227.
20. George M J, et al., Curr Diab Rep 2013; 13(1):72-80.
21. Marsha J D. Am Health Drug Benefits 2011; 4(5):312-322
22. Dhingra A K et al., Antiinflamm Antiallergy Agents Med Chem. 2015; 14(2):81-97.
23. Pithadia A B, Sunita J. Pharmacoligal rep 2011; 63:629-642
24. Zhe Wang, et al., Expert Opin Drug Deliv. 2010 February; 7(2):159-71
25. Sumit G, Wei W, Tsutomu and Satoshi O, Antibodies 2013; 2: 452-500;
26. Beck A, et al., Nat Rev Imm 2010; 10: 345-352
27. Huch M, et al., Nature 2013; 494 (7436): 247-250
28. Mahé M M, et al., Curr Protoc Mouse Biol 2013; 3: 217-240
29. Shimkets R A, Gene Expression Profiling. Meth. in Mol. Biology; 258. Humana Press
30. Raghavachari N, et al., Gene Expression Analaysis, Meth. in Mol. Biol; 1783. Humana Press
31. Kurien B T, et al., Western Blotting, Methods in Molecular Biology; 1312. Humana Press
32. Hnasko R, ELISA, Methods in Molecular Blology, 1318. Humana Press
33. Lipton M S, et al., Mass Spectrometry of Proteins and Peptides, Meth. in Mol. Biology, 492. Humana Press
34. Brady H, Apoptosis Methods and Protocols, Methods in Moelcualr Biology, 282. Humana Press
35. Cheryl L et al., Protein-Protein interactions, Methods in Molecular Biology, 1278. Humana Press
36. Lightfoot Y L, Chen J, Mathews C E, PLOS One. 2011; 6(6):e20617.

1. An isolated antibody capable of binding to human insulin-like growth factor binding protein 3 (IGFBP3) or an antigen binding fragment thereof, wherein the antibody or antigen binding fragment comprises three heavy chain CDRs and three light chain CDRs, the six CDRs selected from:
(a) Yu139-A02: SEQ ID NO:1 (VH CDR1), SEQ ID NO:8 (VH CDR2), SEQ ID NO:17 (VH CDR3), SEQ ID NO:27 (VL CDR1), SEQ ID NO:37 (VL CDR2), and SEQ ID NO:45 (VL CDR3);
(b) Yu139-A03: SEQ ID NO:2 (VH CDR1), SEQ ID NO:9 (VH CDR2), SEQ ID NO:18 (VH CDR3), SEQ ID NO:28 (VL CDR1), SEQ ID NO:38 (VL CDR2), and SEQ ID NO:46 (VL CDR3);
(c) Yu139-B01: SEQ ID NO:3 (VH CDR1), SEQ ID NO:10 (VH CDR2), SEQ ID NO:19 (VH CDR3), SEQ ID NO:29 (VL CDR1), SEQ ID NO:39 (VL CDR2), and SEQ ID NO:47 (VL CDR3);
(d) Yu139-C02: SEQ ID: 4 (VH CDR1), SEQ ID NO:12 (VH CDR2), SEQ ID NO: 21 (VH CDR3), SEQ ID NO:31 (VL CDR1), SEQ ID NO: 38 (VL CDR2), and SEQ ID NO:49 (VL CDR3);
(e) Yu139-D02: SEQ ID NO:5 (VH CDR1), SEQ ID NO:13 (VH CDR2), SEQ ID NO:22 (VH CDR3), SEQ ID NO:32 (VL CDR1), SEQ ID NO:40 (VL CDR2), and SEQ ID NO:50 (VL CDR3);
(f) Yu139-D03: SEQ ID NO:6 (VH CDR1), SEQ ID NO:14 (VH CDR2), SEQ ID NO:23 (VH CDR3), SEQ ID NO:33 (VL CDR1), SEQ ID NO:41 (VL CDR2), and SEQ ID NO:51 (VL CDR3);
(g) Yu139-F02: SEQ ID NO:4 (VH CDR1), SEQ ID NO:12 (VH CDR2), SEQ ID NO:23 (VH CDR3), SEQ ID NO:31 (VL CDR1), SEQ ID NO:38 (VL CDR2), and SEQ ID NO:50 (VL CDR3);
(h) Yu139-G01: SEQ ID NO:4 (VH CDR1), SEQ ID NO:15 (VH CDR2), SEQ ID NO:24 (VH CDR3), SEQ ID NO:34 (VL CDR1), SEQ ID NO:42 (VL CDR2), and SEQ ID NO:52 (VL CDR3);
(i) Yu139-G03: SEQ ID NO:7 (VH CDR1), SEQ ID NO:16 (VH CDR2), SEQ ID NO:25 (VH CDR3), SEQ ID NO:35 (VL CDR1), SEQ ID NO:43 (VL CDR2), and SEQ ID NO:53 (VL CDR3); and
(j) Yu139-H03: SEQ ID NO:4 (VH CDR1), SEQ ID NO:12 (VH CDR2), SEQ ID NO:26 (VH CDR3), SEQ ID NO:36 (VL CDR1), SEQ ID NO:44 (VL CDR2), and SEQ ID NO:54 (VL CDR3).

2. The isolated antibody or antigen binding fragment thereof of claim 1, wherein the VH region and the VL region have the respective sequences of:
(a) Yu139-A02: SEQ ID NO:55 (VH) and SEQ ID NO:66 (VL);
(b) Yu139-A03: SEQ ID NO:56 (VH) and SEQ ID NO:67 (VL);
(c) Yu139-B01: SEQ ID NO:57 (VH) and SEQ ID NO:68 (VL);
(d) Yu139-C02: SEQ ID NO: 59 (VH) and SEQ ID NO:70 (VL);
(e) Yu139-D02: SEQ ID NO: 60 (VH), and SEQ ID NO:71 (VL);
(f) Yu139-D03: SEQ ID NO:61 (VH) and SEQ ID NO:72 (VL);
(g) Yu139-F02: SEQ ID NO:62 (VH) and SEQ ID NO:73 (VL);
(h) Yu139-G01: SEQ ID NO:63 (VH) and SEQ ID NO:74 (VL);
(i) Yu139-G03: SEQ ID NO:64 (VH) and SEQ ID NO:75 (VL); and
(j) Yu139-H03: SEQ ID NO:65 (VH) and SEQ ID NO:76 (VL).

3. The isolated antibody or antigen binding fragment thereof of claim 1, wherein the antibody or antigen binding fragment thereof is humanized.

4. The isolated antibody or antigen binding fragment thereof of claim 1, wherein the antibody or antigen binding fragment thereof is human.

5. The isolated antibody or antigen binding fragment thereof of claim 1, wherein the antibody or antigen binding fragment thereof is a humanized or human IgG1, IgG2 or IgG4 antibody.

6. The isolated antibody or antigen binding fragment thereof of claim 5, wherein the antibody or antigen binding fragment is an IgG1 antibody.

7. The IgG1 antibody of claim 6, wherein the IgG1 antibody is an IgG1 kappa antibody or an IgG1 lambda antibody.

8. The IgG1 antibody of claim 7, wherein the IgG1 antibody is an IgG1 kappa antibody.

9. The IgG1 antibody of claim 7, wherein the IgG1 antibody is an IgG1 lambda antibody.

10. The IgG1 antibody of claim 6, wherein the IgG1 antibody comprises at least one amino acid substitution in the heavy chain constant region (Fc) of the IgG1 antibody, wherein the at least one amino acid substitution is capable of reducing Fc receptor binding or effector cell function of the antibody.

11. The IgG1 antibody of claim 10, wherein the at least one amino acid substitution is capable of reducing Fc receptor binding of the antibody.

12. The IgG1 antibody of claim 10, wherein the at least one amino acid substitution is capable of reducing effector cell function of the antibody.

13. The IgG1 antibody of claim 6, wherein the IgG1 antibody comprises at least one amino acid substitution in the heavy chain constant region selected from the following group: L234A, L235A, T250Q, N297A, and M428L.

14. The isolated antibody or antigen binding fragment thereof of claim 5, wherein the antibody or antigen binding fragment is an IgG2 antibody.

15. The IgG2 antibody of claim 14, wherein the IgG2 antibody is an IgG2 kappa antibody or an IgG2 lambda antibody.

16. The IgG2 antibody of claim 15, wherein the IgG2 antibody is an IgG2 kappa antibody.

17. The IgG2 antibody of claim 15, wherein the IgG2 antibody is an IgG2 lambda antibody.

18. The isolated antibody or antigen binding fragment thereof of claim 5, wherein the antibody or antigen binding fragment is an IgG4 antibody.

19. The IgG4 antibody of claim 18, wherein the IgG4 antibody is an IgG4 kappa antibody or an IgG4 lambda antibody.

20. The IgG4 antibody of claim 19, wherein the IgG4 antibody is an IgG4 kappa antibody.

21. The IgG4 antibody of claim 19, wherein the IgG4 antibody is an IgG4 lambda antibody.

22. A pharmaceutical composition comprising an antibody capable of binding to human insulin-like growth factor binding protein 3 (IGFBP3) or an antigen binding fragment thereof and a pharmaceutically acceptable diluent or excipient,
wherein the antibody or antigen binding fragment comprises three heavy chain CDRs and three light chain CDRs, the six CDRs selected from:
(a) Yu139-A02: SEQ ID NO:1 (VH CDR1), SEQ ID NO:8 (VH CDR2), SEQ ID NO:17 (VH CDR3), SEQ ID NO:27 (VL CDR1), SEQ ID NO:37 (VL CDR2), and SEQ ID NO:45 (VL CDR3);
(b) Yu139-A03: SEQ ID NO:2 (VH CDR1), SEQ ID NO:9 (VH CDR2), SEQ ID NO:18 (VH CDR3), SEQ ID NO:28 (VL CDR1), SEQ ID NO:38 (VL CDR2), and SEQ ID NO:46 (VL CDR3);
(c) Yu139-B01: SEQ ID NO:3 (VH CDR1), SEQ ID NO:10 (VH CDR2), SEQ ID NO:19 (VH CDR3), SEQ ID NO:29 (VL CDR1), SEQ ID NO:39 (VL CDR2), and SEQ ID NO:47 (VL CDR3);
(d) Yu139-C02: SEQ ID: 4 (VH CDR1), SEQ ID NO: 12 (VH CDR2), SEQ ID NO: 21 (VH CDR3), SEQ ID NO:31 (VL CDR1), SEQ ID NO: 38 (VL CDR2), and SEQ ID NO:49 (VL CDR3);
(e) Yu139-D02: SEQ ID NO:5 (VH CDR1), SEQ ID NO:13 (VH CDR2), SEQ ID NO:22 (VH CDR3), SEQ ID NO:32 (VL CDR1), SEQ ID NO:40 (VL CDR2), and SEQ ID NO:50 (VL CDR3);
(f) Yu139-D03: SEQ ID NO:6 (VH CDR1), SEQ ID NO:14 (VH CDR2), SEQ ID NO:23 (VH CDR3), SEQ ID NO:33 (VL CDR1), SEQ ID NO:41 (VL CDR2), and SEQ ID NO:51 (VL CDR3);
(g) Yu139-F02: SEQ ID NO:4 (VH CDR1), SEQ ID NO:12 (VH CDR2), SEQ ID NO:23 (VH CDR3), SEQ ID NO:31 (VL CDR1), SEQ ID NO:38 (VL CDR2), and SEQ ID NO:50 (VL CDR3);
(h) Yu139-G01: SEQ ID NO:4 (VH CDR1), SEQ ID NO:15 (VH CDR2), SEQ ID NO:24 (VH CDR3), SEQ ID NO:34 (VL CDR1), SEQ ID NO:42 (VL CDR2), and SEQ ID NO:52 (VL CDR3);
(i) Yu139-G03: SEQ ID NO:7 (VH CDR1), SEQ ID NO:16 (VH CDR2), SEQ ID NO:25 (VH CDR3), SEQ ID NO:35 (VL CDR1), SEQ ID NO:43 (VL CDR2), and SEQ ID NO:53 (VL CDR3); and
(j) Yu139-H03: SEQ ID NO:4 (VH CDR1), SEQ ID NO:12 (VH CDR2), SEQ ID NO:26 (VH CDR3), SEQ ID NO:36 (VL CDR1), SEQ ID NO:44 (VL CDR2), and SEQ ID NO:54 (VL CDR3).

23. An isolated polynucleotide or plurality of isolated polynucleotides encoding an antibody capable of binding to human insulin-like growth factor binding protein 3 (IGFBP3) or an antigen binding fragment thereof, wherein the antibody or antigen binding fragment comprises three heavy chain CDRs and three light chain CDRs, the six CDRs selected from:
(a) Yu139-A02: SEQ ID NO:1 (VH CDR1), SEQ ID NO:8 (VH CDR2), SEQ ID NO:17 (VH CDR3), SEQ ID NO:27 (VL CDR1), SEQ ID NO:37 (VL CDR2), and SEQ ID NO:45 (VL CDR3);
(b) Yu139-A03: SEQ ID NO:2 (VH CDR1), SEQ ID NO:9 (VH CDR2), SEQ ID NO:18 (VH CDR3), SEQ ID NO:28 (VL CDR1), SEQ ID NO:38 (VL CDR2), and SEQ ID NO:46 (VL CDR3);
(c) Yu139-B01: SEQ ID NO:3 (VH CDR1), SEQ ID NO:10 (VH CDR2), SEQ ID NO:19 (VH CDR3), SEQ ID NO:29 (VL CDR1), SEQ ID NO:39 (VL CDR2), and SEQ ID NO:47 (VL CDR3);
(d) Yu139-C02: SEQ ID: 4 (VH CDR1), SEQ ID NO:12 (VH CDR2), SEQ ID NO: 21 (VH CDR3), SEQ ID NO:31 (VL CDR1), SEQ ID NO: 38 (VL CDR2), and SEQ ID NO:49 (VL CDR3);
(e) Yu139-D02: SEQ ID NO:5 (VH CDR1), SEQ ID NO:13 (VH CDR2), SEQ ID NO:22 (VH CDR3), SEQ ID NO:32 (VL CDR1), SEQ ID NO:40 (VL CDR2), and SEQ ID NO:50 (VL CDR3);
(f) Yu139-D03: SEQ ID NO:6 (VH CDR1), SEQ ID NO:14 (VH CDR2), SEQ ID NO:23 (VH CDR3), SEQ ID NO:33 (VL CDR1), SEQ ID NO:41 (VL CDR2), and SEQ ID NO:51 (VL CDR3);
(g) Yu139-F02: SEQ ID NO:4 (VH CDR1), SEQ ID NO:12 (VH CDR2), SEQ ID NO:23 (VH CDR3), SEQ ID NO:31 (VL CDR1), SEQ ID NO:38 (VL CDR2), and SEQ ID NO:50 (VL CDR3);
(h) Yu139-G01: SEQ ID NO:4 (VH CDR1), SEQ ID NO:15 (VH CDR2), SEQ ID NO:24 (VH CDR3), SEQ ID NO:34 (VL CDR1), SEQ ID NO:42 (VL CDR2), and SEQ ID NO:52 (VL CDR3);
(i) Yu139-G03: SEQ ID NO:7 (VH CDR1), SEQ ID NO:16 (VH CDR2), SEQ ID NO:25 (VH CDR3), SEQ ID NO:35 (VL CDR1), SEQ ID NO:43 (VL CDR2), and SEQ ID NO:53 (VL CDR3); and
(j) Yu139-H03: SEQ ID NO:4 (VH CDR1), SEQ ID NO:12 (VH CDR2), SEQ ID NO:26 (VH CDR3), SEQ ID NO:36 (VL CDR1), SEQ ID NO:44 (VL CDR2), and SEQ ID NO:54 (VL CDR3).

24. An isolated cell comprising a polynucleotide, the polynucleotide having at least one sequence that encodes an antibody capable of binding to human insulin-like growth factor binding protein 3 (IGFBP3) or an antigen-binding fragment thereof, wherein the antibody or antigen-binding fragment comprises three heavy chain CDRs and three light chain CDRs, the six CDRs selected from:
(a) Yu139-A02: SEQ ID NO:1 (VH CDR1), SEQ ID NO:8 (VH CDR2), SEQ ID NO:17 (VH CDR3), SEQ ID NO:27 (VL CDR1), SEQ ID NO:37 (VL CDR2), and SEQ ID NO:45 (VL CDR3);
(b) Yu139-A03: SEQ ID NO:2 (VH CDR1), SEQ ID NO:9 (VH CDR2), SEQ ID NO:18 (VH CDR3), SEQ ID NO:28 (VL CDR1), SEQ ID NO:38 (VL CDR2), and SEQ ID NO:46 (VL CDR3);
(c) Yu139-B01: SEQ ID NO:3 (VH CDR1), SEQ ID NO:10 (VH CDR2), SEQ ID NO:19 (VH CDR3), SEQ ID NO:29 (VL CDR1), SEQ ID NO:39 (VL CDR2), and SEQ ID NO:47 (VL CDR3);
(d) Yu139-C02: SEQ ID: 4 (VH CDR1), SEQ ID NO:12 (VH CDR2), SEQ ID NO: 21 (VH CDR3), SEQ ID NO:31 (VL CDR1), SEQ ID NO: 38 (VL CDR2), and SEQ ID NO:49 (VL CDR3);
(e) Yu139-D02: SEQ ID NO:5 (VH CDR1), SEQ ID NO:13 (VH CDR2), SEQ ID NO:22 (VH CDR3), SEQ ID NO:32 (VL CDR1), SEQ ID NO:40 (VL CDR2), and SEQ ID NO:50 (VL CDR3);
(f) Yu139-D03: SEQ ID NO:6 (VH CDR1), SEQ ID NO:14 (VH CDR2), SEQ ID NO:23 (VH CDR3), SEQ ID NO:33 (VL CDR1), SEQ ID NO:41 (VL CDR2), and SEQ ID NO:51 (VL CDR3);
(g) Yu139-F02: SEQ ID NO:4 (VH CDR1), SEQ ID NO:12 (VH CDR2), SEQ ID NO:23 (VH CDR3), SEQ ID NO:31 (VL CDR1), SEQ ID NO:38 (VL CDR2), and SEQ ID NO:50 (VL CDR3);
(h) Yu139-G01: SEQ ID NO:4 (VH CDR1), SEQ ID NO:15 (VH CDR2), SEQ ID NO:24 (VH CDR3), SEQ ID NO:34 (VL CDR1), SEQ ID NO:42 (VL CDR2), and SEQ ID NO:52 (VL CDR3);
(i) Yu139-G03: SEQ ID NO:7 (VH CDR1), SEQ ID NO:16 (VH CDR2), SEQ ID NO:25 (VH CDR3), SEQ ID NO:35 (VL CDR1), SEQ ID NO:43 (VL CDR2), and SEQ ID NO:53 (VL CDR3); and
(j) Yu139-H03: SEQ ID NO:4 (VH CDR1), SEQ ID NO:12 (VH CDR2), SEQ ID NO:26 (VH CDR3), SEQ ID NO:36 (VL CDR1), SEQ ID NO:44 (VL CDR2), and SEQ ID NO:54 (VL CDR3);
and wherein the isolated cell is a hybridoma, a Chinese hamster ovary (CHO) cell, or a human embryonic kidney cell (HEK293).

25. A vector comprising an isolated polynucleotide, the polynucleotide having at least one sequence that encodes an antibody capable of binding to human insulin-like growth factor binding protein 3 (IGFBP3) or antigen-binding fragment thereof, wherein the antibody or antigen-binding fragment comprises three heavy chain CDRs and three light chain CDRs, the six CDRs selected from:
(a) Yu139-A02: SEQ ID NO:1 (VH CDR1), SEQ ID NO:8 (VH CDR2), SEQ ID NO:17 (VH CDR3), SEQ ID NO:27 (VL CDR1), SEQ ID NO:37 (VL CDR2), and SEQ ID NO:45 (VL CDR3);
(b) Yu139-A03: SEQ ID NO:2 (VH CDR1), SEQ ID NO:9 (VH CDR2), SEQ ID NO:18 (VH CDR3), SEQ ID NO:28 (VL CDR1), SEQ ID NO:38 (VL CDR2), and SEQ ID NO:46 (VL CDR3);
(c) Yu139-B01: SEQ ID NO:3 (VH CDR1), SEQ ID NO:10 (VH CDR2), SEQ ID NO:19 (VH CDR3), SEQ ID NO:29 (VL CDR1), SEQ ID NO:39 (VL CDR2), and SEQ ID NO:47 (VL CDR3);
(d) Yu139-C02: SEQ ID: 4 (VH CDR1), SEQ ID NO:12 (VH CDR2), SEQ ID NO: 21 (VH CDR3), SEQ ID NO:31 (VL CDR1), SEQ ID NO: 38 (VL CDR2), and SEQ ID NO:49 (VL CDR3);
(e) Yu139-D02: SEQ ID NO:5 (VH CDR1), SEQ ID NO:13 (VH CDR2), SEQ ID NO:22 (VH CDR3), SEQ ID NO:32 (VL CDR1), SEQ ID NO:40 (VL CDR2), and SEQ ID NO:50 (VL CDR3);
(f) Yu139-D03: SEQ ID NO:6 (VH CDR1), SEQ ID NO:14 (VH CDR2), SEQ ID NO:23 (VH CDR3), SEQ ID NO:33 (VL CDR1), SEQ ID NO:41 (VL CDR2), and SEQ ID NO:51 (VL CDR3);
(g) Yu139-F02: SEQ ID NO:4 (VH CDR1), SEQ ID NO:12 (VH CDR2), SEQ ID NO:23 (VH CDR3), SEQ ID NO:31 (VL CDR1), SEQ ID NO:38 (VL CDR2), and SEQ ID NO:50 (VL CDR3);
(h) Yu139-G01: SEQ ID NO:4 (VH CDR1), SEQ ID NO:15 (VH CDR2), SEQ ID NO:24 (VH CDR3), SEQ ID NO:34 (VL CDR1), SEQ ID NO:42 (VL CDR2), and SEQ ID NO:52 (VL CDR3);
(i) Yu139-G03: SEQ ID NO:7 (VH CDR1), SEQ ID NO:16 (VH CDR2), SEQ ID NO:25 (VH CDR3), SEQ ID NO:35 (VL CDR1), SEQ ID NO:43 (VL CDR2), and SEQ ID NO:53 (VL CDR3); and
(j) Yu139-H03: SEQ ID NO:4 (VH CDR1), SEQ ID NO:12 (VH CDR2), SEQ ID NO:26 (VH CDR3), SEQ ID NO:36 (VL CDR1), SEQ ID NO:44 (VL CDR2), and SEQ ID NO:54 (VL CDR3);
and optionally wherein the vector is selected from the group consisting of a plasmid, a viral
vector, a non-episomal mammalian vector, an expression vector and a recombinant expression vector.

26. A method of treating a disorder selected from diabetes, diabetic enteropathy, and inflammatory bowel disease, the method comprising:
administering to a subject diagnosed with diabetes, diabetic enteropathy, or inflammatory bowel disease a therapeutically effective amount of a pharmaceutical composition that comprises an antibody or antigen binding fragment thereof,
wherein the antibody or antigen binding fragment comprises three heavy chain CDRs and three light chain CDRs, the six CDRs selected from:
(a) Yu139-A02: SEQ ID NO:1 (VH CDR1), SEQ ID NO:8 (VH CDR2), SEQ ID NO:17 (VH CDR3), SEQ ID NO:27 (VL CDR1), SEQ ID NO:37 (VL CDR2), and SEQ ID NO:45 (VL CDR3);
(b) Yu139-A03: SEQ ID NO:2 (VH CDR1), SEQ ID NO:9 (VH CDR2), SEQ ID NO:18 (VH CDR3), SEQ ID NO:28 (VL CDR1), SEQ ID NO:38 (VL CDR2), and SEQ ID NO:46 (VL CDR3);
(c) Yu139-B01: SEQ ID NO:3 (VH CDR1), SEQ ID NO:10 (VH CDR2), SEQ ID NO:19 (VH CDR3), SEQ ID NO:29 (VL CDR1), SEQ ID NO:39 (VL CDR2), and SEQ ID NO:47 (VL CDR3);
(d) Yu139-C02: SEQ ID: 4 (VH CDR1), SEQ ID NO:12 (VH CDR2), SEQ ID NO: 21 (VH CDR3), SEQ ID NO:31 (VL CDR1), SEQ ID NO: 38 (VL CDR2), and SEQ ID NO:49 (VL CDR3);
(e) Yu139-D02: SEQ ID NO:5 (VH CDR1), SEQ ID NO:13 (VH CDR2), SEQ ID NO:22 (VH CDR3), SEQ ID NO:32 (VL CDR1), SEQ ID NO:40 (VL CDR2), and SEQ ID NO:50 (VL CDR3);
(f) Yu139-D03: SEQ ID NO:6 (VH CDR1), SEQ ID NO:14 (VH CDR2), SEQ ID NO:23 (VH CDR3), SEQ ID NO:33 (VL CDR1), SEQ ID NO:41 (VL CDR2), and SEQ ID NO:51 (VL CDR3);
(g) Yu139-F02: SEQ ID NO:4 (VH CDR1), SEQ ID NO:12 (VH CDR2), SEQ ID NO:23 (VH CDR3), SEQ ID NO:31 (VL CDR1), SEQ ID NO:38 (VL CDR2), and SEQ ID NO:50 (VL CDR3);
(h) Yu139-G01: SEQ ID NO:4 (VH CDR1), SEQ ID NO:15 (VH CDR2), SEQ ID NO:24 (VH CDR3), SEQ ID NO:34 (VL CDR1), SEQ ID NO:42 (VL CDR2), and SEQ ID NO:52 (VL CDR3);
(i) Yu139-G03: SEQ ID NO:7 (VH CDR1), SEQ ID NO:16 (VH CDR2), SEQ ID NO:25 (VH CDR3), SEQ ID NO:35 (VL CDR1), SEQ ID NO:43 (VL CDR2), and SEQ ID NO:53 (VL CDR3); and
(j) Yu139-H03: SEQ ID NO:4 (VH CDR1), SEQ ID NO:12 (VH CDR2), SEQ ID NO:26 (VH CDR3), SEQ ID NO:36 (VL CDR1), SEQ ID NO:44 (VL CDR2), and SEQ ID NO:54 (VL CDR3);
wherein the antibody or antigen-binding fragment thereof is capable of binding to human IGFBP3 and preventing binding of IGFBP3 to transmembrane protein 219 (TMEM219).

27. The method of claim 26, wherein the antibody or antigen binding fragment has the respective VH region and the VL region sequences of:
(a) Yu139-A02: SEQ ID NO:55 (VH) and SEQ ID NO:66 (VL);
(b) Yu139-A03: SEQ ID NO:56 (VH) and SEQ ID NO:67 (VL);
(c) Yu139-B01: SEQ ID NO:57 (VH) and SEQ ID NO:68 (VL);
(d) Yu139-C02: SEQ ID NO: 59 (VH) and SEQ ID NO:70 (VL);
(e) Yu139-D02: SEQ ID NO: 60 (VH), and SEQ ID NO:71 (VL);
(f) Yu139-D03: SEQ ID NO:61 (VH) and SEQ ID NO:72 (VL);
(g) Yu139-F02: SEQ ID NO:62 (VH) and SEQ ID NO:73 (VL);
(h) Yu139-G01: SEQ ID NO:63 (VH) and SEQ ID NO:74 (VL);
(i) Yu139-G03: SEQ ID NO:64 (VH) and SEQ ID NO:75 (VL); and
(j) Yu139-H03: SEQ ID NO:65 (VH) and SEQ ID NO:76 (VL).

28. The method of claim 26, wherein the antibody or antigen-binding fragment thereof is humanized or human.

29. The method of claim 28, wherein the antibody or antigen-binding fragment thereof is a humanized or human IgG1, IgG2 or IgG4 antibody.

30. The method of claim 26, wherein the disorder is diabetes.

31. The method of claim 30, wherein the disorder is type 1 diabetes.

32. The method of claim 30, wherein the disorder is type 2 diabetes.

33. The method of claim 26 wherein the disorder is diabetic enteropathy.

34. The method of claim 26, wherein the disorder is inflammatory bowel disease.

35. The method of claim 34, wherein the disorder is Crohn's disease.

36. The method of claim 34, wherein the disorder is ulcerative colitis.

37. A method of inhibiting the binding of human IGFBP3 to TMEM219, the method comprising:
contacting human IGFBP3 in vivo with an antibody or antigen-binding fragment thereof, where the antibody or antigen-binding fragment comprises three heavy chain CDRs and three light chain CDRs, the six CDRs selected from:
(a) Yu139-A02: SEQ ID NO:1 (VH CDR1), SEQ ID NO:8 (VH CDR2), SEQ ID NO:17 (VH CDR3), SEQ ID NO:27 (VL CDR1), SEQ ID NO:37 (VL CDR2), and SEQ ID NO:45 (VL CDR3);
(b) Yu139-A03: SEQ ID NO:2 (VH CDR1), SEQ ID NO:9 (VH CDR2), SEQ ID NO:18 (VH CDR3), SEQ ID NO:28 (VL CDR1), SEQ ID NO:38 (VL CDR2), and SEQ ID NO:46 (VL CDR3);
(c) Yu139-B01: SEQ ID NO:3 (VH CDR1), SEQ ID NO:10 (VH CDR2), SEQ ID NO:19 (VH CDR3), SEQ ID NO:29 (VL CDR1), SEQ ID NO:39 (VL CDR2), and SEQ ID NO:47 (VL CDR3);
(d) Yu139-C02: SEQ ID: 4 (VH CDR1), SEQ ID NO:12 (VH CDR2), SEQ ID NO: 21 (VH CDR3), SEQ ID NO:31 (VL CDR1), SEQ ID NO: 38 (VL CDR2), and SEQ ID NO:49 (VL CDR3);
(e) Yu139-D02: SEQ ID NO:5 (VH CDR1), SEQ ID NO:13 (VH CDR2), SEQ ID NO:22 (VH CDR3), SEQ ID NO:32 (VL CDR1), SEQ ID NO:40 (VL CDR2), and SEQ ID NO:50 (VL CDR3);
(f) Yu139-D03: SEQ ID NO:6 (VH CDR1), SEQ ID NO:14 (VH CDR2), SEQ ID NO:23 (VH CDR3), SEQ ID NO:33 (VL CDR1), SEQ ID NO:41 (VL CDR2), and SEQ ID NO:51 (VL CDR3);
(g) Yu139-F02: SEQ ID NO:4 (VH CDR1), SEQ ID NO:12 (VH CDR2), SEQ ID NO:23 (VH CDR3), SEQ ID NO:31 (VL CDR1), SEQ ID NO:38 (VL CDR2), and SEQ ID NO:50 (VL CDR3);
(h) Yu139-G01: SEQ ID NO:4 (VH CDR1), SEQ ID NO:15 (VH CDR2), SEQ ID NO:24 (VH CDR3), SEQ ID NO:34 (VL CDR1), SEQ ID NO:42 (VL CDR2), and SEQ ID NO:52 (VL CDR3);
(i) Yu139-G03: SEQ ID NO:7 (VH CDR1), SEQ ID NO:16 (VH CDR2), SEQ ID NO:25 (VH CDR3), SEQ ID NO:35 (VL CDR1), SEQ ID NO:43 (VL CDR2), and SEQ ID NO:53 (VL CDR3); and
(j) Yu139-H03: SEQ ID NO:4 (VH CDR1), SEQ ID NO:12 (VH CDR2), SEQ ID NO:26 (VH CDR3), SEQ ID NO:36 (VL CDR1), SEQ ID NO:44 (VL CDR2), and SEQ ID NO:54 (VL CDR3).

38. The method of claim 37, wherein the antibody or antigen binding fragment has the respective VH region and the VL region of:
(a) Yu139-A02: SEQ ID NO:55 (VH) and SEQ ID NO:66 (VL);
(b) Yu139-A03: SEQ ID NO:56 (VH) and SEQ ID NO:67 (VL);
(c) Yu139-B01: SEQ ID NO:57 (VH) and SEQ ID NO:68 (VL);
(d) Yu139-C02: SEQ ID NO: 59 (VH) and SEQ ID NO:70 (VL);
(e) Yu139-D02: SEQ ID NO: 60 (VH), and SEQ ID NO:71 (VL);
(f) Yu139-D03: SEQ ID NO:61 (VH) and SEQ ID NO:72 (VL);
(g) Yu139-F02: SEQ ID NO:62 (VH) and SEQ ID NO:73 (VL);
(h) Yu139-G01: SEQ ID NO:63 (VH) and SEQ ID NO:74 (VL);
(i) Yu139-G03: SEQ ID NO:64 (VH) and SEQ ID NO:75 (VL); and
(j) Yu139-H03: SEQ ID NO:65 (VH) and SEQ ID NO:76 (VL).

39. The method of claim 37, wherein the antibody or antigen-binding fragment thereof is humanized or human.

40. The method of claim 39, wherein the antibody or antigen-binding fragment thereof is a humanized or human IgG1, IgG2 or IgG4 antibody.
